(19) United States
(12) Patent Application Publication
(10) Pub. No.: US 2022/0096539 A1
(43) Pub. Date: Mar. 31, 2022

(54) SALT NANOPARTICLES AND COMPOSITIONS AND METHODS OF USE THEREOF

(71) Applicants: University of Georgia Research Foundation, Inc., Athens, GA (US); The University of North Carolina at Chapel Hill, Chapel Hill, NC (US)

(72) Inventors: Jin Xie, Athens, GA (US); Wen Jiang, Athens, GA (US); Trever Todd, Fountain Valley, CA (US); Zibo Li, Chapel Hill, NC (US)

(21) Appl. No.: 17/424,109

(22) PCT Filed: Jan. 17, 2020
(86) PCT No.: PCT/US20/14122

§ 371 Date: Jul. 19, 2021

Related U.S. Application Data
(60) Provisional application No. 62/794,350, filed on Jan. 18, 2019.

(51) Int. Cl. A61K 33/14; A61K 39/39; A61K 45/06; A61K 9/50; A61P 35/00; A61K 39/00; A61K 39/395

(52) U.S. Cl. CPC A61K 33/14; A61K 39/39; A61K 45/06; A61K 9/5015; A61K 2039/505; A61K 39/0011; A61K 39/3955; A61K 2039/55505; A61K 2039/5152; A61P 35/00

(57) ABSTRACT

Particles formed from an alkai metal or alkaline earth metal and halide, for example, sodium and chloride, are provided. The particles can have a hydrophilic coating or external layer, formed of, for example, a polyether-lipid conjugate. In preferred embodiments, the lipid is a phospholipid such as a phosphoethanolamine, and the polyether is a polyethylene glycol such as a PEG amine. Methods making the particles by, for example, a microemulsion reaction, are also provided. Pharmaceutical compositions including a plurality of particles and a pharmaceutically acceptable carrier are also disclosed. Typically the compositions include an effective amount of particles to treat a disease or condition, particularly cancer, in a subject in need thereof. The particles are typically nanoparticles, for example, between about 10 nm and 250 nm and can be monodisperse.

### CROSS-REFERENCE TO RELATED APPLICATIONS

This application claims the benefit of and priority to U.S. Provisional Application No. 62/794,350 filed Jan. 18, 2019, which is hereby incorporated by reference in its entirety

### STATEMENT REGARDING FEDERALLY SPONSORED RESEARCH

This invention was made with government support under Grant NSF1552617 awarded by the National Science Foundation and R01EB022596 by the National Institutes of Health. The government has certain rights in the invention.

### FIELD OF THE INVENTION

The invention is generally directed to particle compositions and methods of use thereof, particularly for the treatment of cancer.

### BACKGROUND OF THE INVENTION

Cancer therapies are often severely limited by significant side effects due to non-specific tissue toxicity, and identification of new agents that are selectively toxic to cancer cells or selectively sensitize tumors to treatment is an important goal in cancer research. For example, in one area, investigation has concentrated on applying the specific binding activity of monoclonal antibodies to the development of tumor-specific therapies. Select antibodies such as trastuzumab (Herceptin®), rituximab (Rituxan®), and cetuximab (Erbitux®) have received approval for use in human cancer therapy, but all lack the ability to penetrate into cancer cells and are therefore limited to attacking targets located on the external surface of tumor cells.

Particulate vaccines is another promising area that provides the ability to tune prophylactics and therapeutics against a wide variety of conditions including cancer. Vesicular and solid biodegradable polymer platforms, exemplified by liposomes and polyesters, respectively, are two of the most ubiquitous platforms in vaccine delivery studies. Immunization with poly(lactide-co-glycolide) (PLGA) nanoparticles elicits prolonged antibody titers compared to liposomes and alum. The magnitude of the cellular immune response is highest in animals vaccinated with PLGA, which also shows a higher frequency of effector-like memory T-cell phenotype, leading to an effective clearance of intracellular bacteria. The difference in performance of these two common particulate platforms is shown not to be due to material differences but appears to be connected to the kinetics of antigen delivery. Liposomes are easily modified for encapsulation of small hydrophilic molecules, and even proteins. However, the stability of these formulations and the release profiles of encapsulated agents are not easily controlled. Biodegradable solid particles, on the other hand, such as those fabricated from poly(lactic-co-glycolic acid) (PLGA), are highly stable and have controllable release characteristics, but pose complications for facile encapsulation and controlled release of therapeutic cytokines or for combinatorial delivery.

There remains a need to improved technologies for cancer treatment.

Thus, it is an object of the invention to provide compositions and methods for use in cancer treatment.

### SUMMARY OF THE INVENTION

Particles formed of a salt formed from an alkai metal or alkaline earth metal and halide, also referred to salt particles, and methods of use thereof are provided. In preferred embodiments, the salt particles are sodium chloride (NaCl) particles, preferably nanoparticles. The particles can be, for example, cubic nanoparticles. In particular embodiments, alkai metal or alkaline earth metal and halide (e.g., sodium and chloride) particles have a molar ratio of sodium:chloride of about 1:1.

The salt particles can have a hydrophilic coating or external layer, formed of, for example, amphiphilic polymer, protein, lipid, or conjugate thereof such as a polyether-lipid conjugate. In preferred embodiments, the lipid is a phospholipid such as a phosphoethanolamine, and the polyether is a polyethylene glycol such as a PEG amine.

Pharmaceutical compositions including a plurality of the same or different salt particles and a pharmaceutically acceptable carrier are also provided. In some embodiments, the compositions include particles having an average hydrodynamic size of between about 10 nm and about 500 nm, or between about 25 nm and about 300 nm, or between about 50 nm and 150 nm, between about 75 nm and about 125 nm, ±5%, 10%, 15%, 20%, or 25%. The particles in the composition can be monodisperse.

Methods of making salt particles, and the salt particles formed according to such methods are also provided. For example, in some embodiments, NaCl particles are formed by a microemulsion reaction. The microemulsion reaction can include, for example, adding molybdenum (V) chloride to a solvent solution including a solvent, a reductant, a surfactant, and sodium oleate. The reaction can be free from water. In particular embodiments, the solvent is a mixture of hexane and ethanol. The reductant is hexadecanediol or tetradecanediol and the surfactant is oleylamine or oleic acid. The method can include the step of adding a hydrophilic coating or external layer formed by mixing the particles and a lipid-polyether conjugate together in a solvent and removing the solvent.

The pharmaceutical compositions can include a therapeutically effective amount of any of the salt particles. For example, in some embodiments, the compositions include an effective amount of particles to reduce mitochondrial oxygen consumption rate (OCR), reduce mitochondrial respiration rate (MSR), decrease intracellular ATP level, increase the ROS level, increase levels of JNK. ERK, and/or p38 phosphorylation, increase lipid peroxidation, increase DNA damage, release of cytochrome c, increase of caspase-3 activity, increase caspase-1 activity, increase cell swelling and/or bleb formation, induce cell rupture and/or complete osmotic lysis, increase NLRP3 inflammasome induction, increase GSDMD N-terminal fragment release, elevate IL-1β secretion, increase intracellular K⁺ level, increased presentation/secretion of calreticulin (CRT), increased presentation/secretion of adenosine triphosphate (ATP), h increased presentation/secretion of high mobility group box 1 (HMGB1), or a combination thereof in tumor cells and/or cancer cells. In some embodiments the amount of salt particles is effective to increase apoptosis, necrosis, and/or pyroptosis of tumor and/or cancer cells. Preferably, the composition is administered in an amount or/manner that the foregoing are altered or effected to a greater degree in tumor and/or cancer cells than non-tumor or non-cancer (e.g., control, or health) cells.

In some embodiments, the pharmaceutical composition is in a dosage form suitable for administration of about 0.1 mg/kg to about 1,000 mg/kg, or about 1 mg/kg to about 100 mg/kg, or about 5 mg/kg to about 50 mg/kg to a subject in need thereof.

Methods of treatment are also provided. The disclosed compositions are particularly useful for the treatment of cancer. Such methods typically include administrating to a subject in need thereof an effective amount of the disclosed particles. The subject can have, for example, a bone, bladder, brain, breast, cervical, colo-rectal, esophageal, kidney, liver, lung, nasopharyngeal, pancreatic, prostate, skin, stomach, or uterine cancer. Any suitable method of administration can be utilized, however, a preferred method is injection or infusion. In some embodiments, the administration is local to the site in need of treatment, for example adjacent to, or directly into, a tumor. In a particular embodiments, particles are administered by intravesical instillation into, for example, the bladder to treat, for example bladder cancer.

Methods of making antigen and antigens formed according to the methods are also provided. The methods typically include contacting cancer cells with an effective amount of salt particles to induce death of the cells.

In preferred embodiments, the cancer cells exhibit increased expression or secretion or release of one or more damage-associated molecular pattern (DAMP) molecules. Preferred DAMP molecule(s) include, for example, calreticulin (CRT), adenosine triphosphate (ATP), high mobility group box 1 (HMGB1), and combinations thereof. Typically, the contacting occurs in vitro or ex vivo. The cancer cells can be, for example, isolated from a subject in need of cancer treatment or prevention.

The antigen formed according to the disclosed methods can be the dying or dead cells, or a lysate, extract, fraction, isolate, or collection of secreted factors thereof.

Vaccination methods utilizing antigens formed according to the methods herein are also provided. The vaccinations are typically for the treatment or prevention of cancer. The methods typically include administering a subject in need thereof an effective amount of antigen to increase or induce an immune response to the antigen. In some embodiments, the subject is also administered salt particles, an adjuvant, or a combination thereof. Any combination of the antigen, the particles, and the adjuvant can be part of the same or different admixtures. Any combination of the antigen, the salt particles, and the adjuvant can be administered together or separately. In some embodiments, the subject has cancer.

Combination therapies including administration of salt particles, for example NaCl nanoparticles, in combination with one or more additional therapeutic agents are also provided. For example, in some embodiments, the additional agent is an immune checkpoint inhibitor, a chemotherapeutic agent, or a combination thereof. Immune checkpoint inhibitors include, but are not limited to, PD-1 antagonists, CTLA4 antagonists, and combinations thereof. In particular embodiments, the PD-1 antagonist and/or CTLA antagonist is an antibody or antigen binding fragment thereof. The particles and the additional active agent can be administered to the subject at different times or the same time, and in the same pharmaceutical composition or different pharmaceutical compositions.

### BRIEF DESCRIPTION OF THE DRAWINGS

FIG. 1A is a TEM image of NaCl nanoparticles (SCNPs) including an inset with a zoomed-in TEM image of a single NaCl nanoparticle. Scale bars, 100 nm. FIG. 1B is an x-ray diffusion (XRD) pattern of as-synthesized NaCl and NaCl standard (PDF: 00-005-0628). FIGS. 1C and 1D are energy-dispersive (EDS) x-ray spectrum of NaCl nanoparticles. FIGS. 1E and 1F are plots showing the release profiles (% release v. time (hrs.)) for Na⁺ (1E) and Cl⁻ (1F), examined with phospholipid coated NaCl nanoparticles (PSCNPs) in ammonium acetate buffer solutions with a pH value of either 7.0 or 5.5. The quantification was based on SBFI-AM and MQAE fluorescence intensity changes (mean±s.d., n=6). FIG. 1G is a FT-IR spectra (T (%)) vs. wavenumber (cm⁻¹)) of as-synthesized SCNPs, precursors oleylamine, sodium oleate, and 1,2-tetradecanediol. FIG. 1H is a plot showing a dynamic light scattering (DLS) analysis of SCNPs as well as PSCNPs. The hydrodynamic size of SCNPs in hexane was 84.6±9.8 nm. After phospholipid coating, the hydrodynamic size of PSCNPs in water was 98.0±13.1 nm. FIG. 1I is a plot showing the zeta potential of PSCNPs in D.I. water, +9.7 mV. FIGS. 1J-1M are a series of representative TEM images of SCNPs of different sizes. Scale bars, 100 nm. FIGS. 1N-1P are a series of representative SEM images of SCNPs of different sizes. Scale bars, 100 nm.

FIG. 2A is a bar graph showing cell viability, measured by MTT assays in PC-3 cells at 24 h. FIG. 2B is a bar graph showing mitochondrial membrane potential changes (ΔΨm), assessed by JC-1 staining at 4 h. JC-1 red/green (aggregate/monomer) fluorescence intensity ratio was significantly decreased when PC-3 cells were incubated with 160.0 μg/mL PSCNPs. The statistics was based on the analysis on 5000 individual cells (mean t s.d.). FIG. 2C is a line graph showing oxygen consumption rate (OCR) changes, assessed by Seahorse mitochondrial stress assay. The readings were normalized to the baseline OCR prior to PSCNPs injection. Relative to PBS treated cells, the 6 h basal was decreased by 2.3±3.3, 18.1±5.0, 47.9±5.4% in cells treated with 52.5, 105.0, and 160.0 μg/mL of PSCNPs (mean±s.d., n=6), respectively. FIG. 2D is a bar graph showing MSR changes, assessed in support of ATP production (i.e. oxidative phosphorylation) at 6 h (mean±s.d., n=6). MSR was decreased from 30.0±1.1 pmol/min in normal cells to 10.0±0.7, 3.3±1.2, and 0.9±1.0 pmol/min, respectively, in cells treated with PSCNPs at 52.5, 105.0, or 160.0 μg/mL. FIG. 2E is a bar graph showing intracellular ATP levels, analyzed by Luminescent ATP Detection Assay at 4 h. The readings were normalized to that of PBS treated cells. A higher PSCNP concentration was associated with lower ATP production. FIG. 2F is a bar graph showing intracellular ROS levels, analyzed by DCFH-DA assay at 4 h. The readings were normalized to PBS treated cells. A dose-dependent ROS production was observed after PSCNP treatment. FIG. 2G is a bar graph showing quantitative analysis of the impact of PSCNPs on JNK, ERK and p38 protein kinases, assessed by Western blotting analysis. PC-3 cells were incubated with PSCNPs (160 μg/mL) for 24 before the analysis. PBS, NaCl salt (160 μg/mL), and degraded PSCNPs (by aging in water for 24 h before experiments, 160 μg/mL) were used as controls. FIG. 2H is a bar graph showing lipid peroxidation, assessed by Lipid Peroxidation Sensor Assay at 24 h. FIG. 2I is a bar graph showing DNA damage at 24 h, analyzed by γH2AX staining. FIG. 2J is a bar graph showing cytochrome c release, analyzed by ApoTrack™ Cytochrome c Apoptosis ICC Antibody Kit at 24 h. Fluorescence signals were quantified by ImageJ (mean±s.d., n=1000 cells). FIG. 2K is a bar graph showing caspase-3 activity, assessed by anti-caspase-3 antibody staining at 24 h (mean t s.d., n=5000 cells). FIG. 2L is a matched pair of bar graphs showing Fold of Change (top) and Dead cell (%) at 1, 2, 4, 6, and 12 hrs. of a kinetic cytotoxicity study. PSCNPs were incubated with PC-3 cells at a dose range of 0-160 μg/mL and the cell viability between 0 and 12 h was assessed by Live/Dead (Calcein AM/PI) assay. FIG. 2M is a bar graph showing cell viability, analyzed by MTT assay. PSCNPs (160.0 μg/mL) were pre-aged in PBS for 1, 3 or 8 h before incubating with PC-3 cells. Standard MTT assays were conducted at 24 h of cell incubation. PBS, NaCl salt (160.0 μg/mL), and the surface coated material, DSPE-PEG2000 amine (phosphatelipid), were studied for comparison. The results are expressed as mean±S.E.M. *, p<0.05. FIG. 2N is a histogram of PSCNPs cell uptake and intracellular degradation as measured by comparing intracellular Rhodamine B signals among RB-PSCNPs (160.0 μg/mL), NaCl (160.0 μg/mL), and PBS treated cells at 1, 2, 4 and 6 h. The results are represented as Rhodamine B intensity per cell (n=5000 cells). FIG. 2O is a histogram comparing intracellular Na+ concentrations at different time points. The results are represented as SBFI-AM intensity per cell (n=5000 cells). PBS and NaCl salt (160.0 μg/mL) treated cells were studied as controls. FIG. 2P is a histogram comparing intracellular Cl⁻ concentrations at different time points. The results are represented as MQAE intensity per cell (n=5000 cells). PBS and NaCl salt (160.0 μg/mL) treated cells were studied as controls. FIG. 2Q is a plot showing cell viability, measured by MTT assays in PC-3 cells after 6 and 24 h incubation with PSCNPs at the concentration from 26.3 to 320 μg mL⁻¹. (*p<0.05 compare to PBS treated control cells). FIG. 2R is a bar graph illustrating the cellular uptake of NaCl NPs in cancer cell lines, T24 and UMUC2, and normal cell lines, K1970 and HPrEC.

FIG. 3A is a plot showing cell volume changes, based on the statistics of 5000 cells. The 98% quantile of PBS treated cells (37500 pixels) was set as the threshold. Cells having areas above the threshold were marked as red dots, and those below the threshold marked black. Concentration- and time-dependent cell expansion was observed after PSCNP treatment. FIG. 3B is a bar graph of cell necrosis, assessed by EthD-III staining. FIG. 3C is a line graph showing LDH release, assessed by LDH Assay Kit-WST. PC-3 cells were incubated with PSCNPs or NaCl salt (6.25-200 μg/mL) for 6 h. All the readings were normalized to PBS treated control cells (*p<0.05). FIG. 3D is a histogram showing the results of flow cytometry to evaluate caspase-1 activation after PSCNP treatment (160.0 μg/mL). FIG. 3E is a bar graph showing LDH release to assess the suppression of glycine and Ac-YVAD-cmk to cell necrosis. PC-3 cells were pre-incubated with necrotic cell death inhibitor glycine or caspase-1 inhibitor Ac-YVAD-cmk for 1 h. PSCNPs (200 μg/mL) were then incubated with cells for 1.5, 3 and 6 h. LDH release was measured by LDH Assay Kit-WST (*p<0.05). FIG. 3F is a scatter plot and FIG. 3G is a line graph each showing cell volume changes over time. PC-3 cells were incubated with PSCNPs at varied concentrations (52.5-160 μg/mL). Cell areas (in pixels) at 30, 60, and 90 min were analyzed and compared (n=5000 cells). FIG. 3H is a schematic representation of the computational model for ion concentration induced cell cytolysis. FIGS. 3I and 3J are line graphs showing the relationship between the membrane tension and ion concentration gradient across the membrane. FIG. 3J is a blow-up of the boxed area in the lower left of FIG. 3I. FIG. 3I illustrates, for different size of cells, the critical concentration gradients (Ac) upon which the plasma membrane begins to rupture (FIG. 3I, squared area in the top right). By curve fitting these data points, an interesting curve used to predict the critical concentration for 25 μm cells was obtained. FIG. 3K is a bar graph showing the impact of PSCNPs on NLRP3 inflammasome activation and GSDMD-N terminal release. Results are ImageJ analysis of the Western blotting of NLRP3 and GSDMD-N levels relative to the PBS controls in PC-3 cells treated with 40.0 and 80.0 μg/mL PSCNPs for 2 h. FIG. 3L is a bar graph showing IL-1β release, analyzed by ELISA. PC-3 were incubated with PSCNPs (100 and 200 μg/mL) for 2 h (*p<0.05 compared to PBS treated control group). FIG. 3M is a histogram comparing intracellular K+ concentrations at different time points (1, 2, 4 and 6 h of PSCNPs incubation). The results are represented as PBFI-AM intensity per cell (n=5000 cells). PBS and NaCl salt (160.0 μg/mL) treated cells were studied as controls. FIG. 3N is a bar graph showing plasma membrane potential changes. PC-3 cells were incubated with PSCNPs of different concentrations for 150 min before the staining for DiBAC₄(3). The fluorescence intensity was analyzed in ImageJ based on the imaging results and normalized to PBS treated control cells. (mean t s.d., n=5000 cells). A dose-dependent decrease of DiBAC₄(3) fluorescence intensity was observed, indicating PSCNP-induced membrane hyperpolarization. FIG. 3O is a bar graphs showing LDH release to assess the suppression of glycine and Ac-YVAD-cmk to cell necrosis. PC-3 cells were pre-incubated with necrotic cell death inhibitor glycine or caspase-1 inhibitor Ac-YVAD-cmk for 1 h. PSCNPs (160 and 320 μg mL-1) were then incubated with cells for 6 h. LDH release was measured by LDH Assay Kit-WST (*p<0.05).

FIG. 4 is an illustration of proposed mechanisms behind NaCl NP-induced cell death.

FIGS. 5A-5I are bar graphs showing cytotoxicity (cell viability vs. PSCNPs μg/mL) against a panel of cell lines [U87MG, IC₅₀=50.8 (5A); 4T1, IC₅₀=90.8 (5B); HT29, IC₅₀=86.2 (5C); B16-F10, IC₅₀=107.0 (5D); SGC7901, IC₅₀=140.2 (5E); A549, IC₅₀=151.5 (5F); RAW246.7. IC₅₀=251.0 (5G); Human primary prostate epithelial cells (HPrECs) (5H); Mouse spermatagonial stem cells (C18) (5I)] measured by MTT assays. While cancer cells were effectively killed by PSCNPs, normal cells were highly resistant. IC₅₀ values were determined by DoseResp of Origin 9. FIG. 5J is a line graph showing the correlation between intracellular sodium content [Na⁺]ᵢₙₜ (pg/cell) and IC₅₀. [Na⁺]ᵢₙₜ of each cell line as determined using a Na⁺ electrode. K-means clustering algorithm was used to evaluate the correlation between [Na⁺]ᵢₙₜ and IC₅₀. FIGS. 5K and 5L are graphs showing in vivo PC-3 tumor therapy outcomes: tumor volume (mm²) (5K) and body weight (g) (5L) as over time (days). PSCNPs or saline with the same NaCl dose (9 mg/mL, 50 μL) were i.t. injected into PC-3 tumor xenografts (n=5). Tumors were dissected 16 days after the treatment. FIG. 5M is a bar graph showing excised tumor weight (*p<0.05 compared to saline control group). FIGS. 5N-5V are bar graphs showing in vivo tumor therapy (tumor volume (mm²) (5N-5Q, 5V)) and tumor growth curves (weight (g)) (5R-5U) for other tumor models, including U87MG (human glioblastoma astrocytoma) (5N, 5R), B16F10 (mouse melanoma) (5O,5S), SCC VII (mouse head and neck squamous carcinoma) (5P, 5T, 5V), and UPPL-1541 (mouse bladder cancer cell line) (5Q, 5U) (*P<0.05). FIG. 5W is a graph showing animal survival curves in SCC VII tumor model (*p<0.05).

FIGS. 6A and 6B are bar graphs showing that ATP release from B16F10 (6A) and SCC VII (6B) cells killed by NaCl NPs. It was found that the ATP release was in a time- and dose-dependent manner (13.2-320 μg/mL) (*p<0.05 compared to saline treated control group). FIGS. 6C and 6D are graphs showing high-mobility group box 1 protein (HMGB-1) release from B16F10 (6C) and SCC VII (6D) cells after NaCl NPs treatment at 24 h. NaCl salt was used as a negative control. PBS treated cells was also used as a control (*p<0.05 compared to PBS treated control group). FIGS. 6E and 6F are histograms of CRT presentation on dying B16F10 and SCC VII cells. Cells were treated with 160 μg mL⁻¹ PSCNPs for 2 h.

FIGS. 7A-7D illustrate an in vivo anti-B16F10 vaccination approach induced by NaCl NPs treatment. FIG. 7A is an animal experimentation graph showing one time vaccination of dying B16F10 cells (2×10⁵) generated by Freeze and Thaw (F/T) or PSCNPs treatment, followed by subcutaneous (SC) injecting live B16F10 cells (2×10⁵) on the contralateral side. Tumors were collected on Day 22. FIG. 7B is a line graph showing B16F10 tumor growth in the contralateral flank (*p<0.05 compared to PBS treated control group). FIGS. 7C and 7D are the graphs showing in vivo anti-SCC VII vaccination approach induced by NaCl NPs treatment. FIG. 7C is an animal experimentation graph showing 2 rounds of vaccination of dying SCC VII cells (2×105) generated by NaCl NPs treatment, with 6 days apart, followed by SC injecting live SCC cells (2×10W) on the contralateral side. Tumors were collected on Day 24. FIG. 7D is a line graph showing SCC VII rumor growth in the contralateral flank (*p<0.05 compared to PBS treated control group).

FIGS. 8A-8B illustrate the antitumor efficacy of NaCl NPs in a SCC VII bilateral tumor model. FIG. 8A is a schematic illustration showing the experimental design. Cells were mixed with Matrigel for tumor inoculation. 1×10⁶ cells were inoculated on the right flank of the animal as the primary tumor, while 0.5×10⁶ SCC cells were inoculated on the left flank as the secondary tumor. Treatment of NaCl NPs or Saline was performed on Day 0. Each mouse in NPs group was injected 1.35 mg NaCl NPs in 50 μL saline. Saline treated group was used as a negative control. Tumor bearing mice w/o any treatment were used as an untreated control. FIG. 8B is a line graph showing the secondary tumor growth (*p<0.05 compared to NaCl NPs treated control group).

FIG. 9A is a schematic illustration showing the experimental design. Bilateral-tumor-bearing C3H mice (n=5) were i.t. injected with one dose of saline or PSCNPs (27 mg mL-1, 50 μL) in the primary tumor 14 days after tumor inoculation. The tumors, spleen, tumor-draining lymph nodes (TDLNs), and blood were collected on day 3, 7, and 12 for flow cytometry analysis. FIGS. 9B (saline) and 9C (PSCNPs) are tumor growth curves for the secondary tumors. FIG. 9D is a bar graph showing a summary of the secondary tumor weight on Day 12 (*p<0.05 compared to saline group). FIGS. 9E and 9F are line graphs showing animal body weight changes of saline (9E) and PSCNPs (9F) groups.

FIGS. 10A-10W are plots showing flow cytometry analysis of leucocyte profiles in blood and tissue samples on day 3, 7, and 12, including: CD8+ T cells (10A-10E), CD8+IFN-γ+ T cells (10F-10J), CD4+Foxp3+ T cells (Tregs) (10K-10O), CD8+ T cells/Treg ratio (10P-10T); CD80+CD86+DCs (10U) and CCR7+CD80+CD86+DCs (10V) in the primary tumors; and B cells (B220+CD19+) in the blood (10W). The study was performed in SCC VII bilateral tumor models. (*p<0.05).

FIGS. 11A and 11B are plots of tumor growth curves (11A) and body weight (11B) illustrating the therapy results of NaCl NPs, tested in C57/BL6 mice bearing BBN963 tumors. PSCNPs were administered intratumorally (3.25 mg in 50 μL, three doses, given three days apart). Anti-PD-1 antibodies were given intraperitoneally (10 mg/kg, three doses, given three days apart).

FIGS. 12A-12D are bar graphs showing ATP release, tested with human and murine bladder cancer cell lines (T-24 (12A), UMUC2 (12B), UPPL-1541 (12C), and BBN963 (12D)) at different PSCNPs concentrations (31.25-250 μg/ml). FIG. 12E is a bar graph showing CRT presentation, tested with bladder cancer cell lines.

### DETAILED DESCRIPTION OF THE INVENTION

### I. Definitions

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth and division, invasion of adjacent tissues, and often metastasizes to other locations of the body.

As used herein, the term “antineoplastic” refers to a composition, such as a drug or biologic, that can inhibit or prevent cancer growth, invasion, and/or metastasis.

As used herein, the term “biocompatible” as used herein refers to one or more materials that are neither themselves toxic to the host (e.g., an animal or human), nor degrade (if the material degrades) at a rate that produces monomeric or oligomeric subunits or other byproducts at toxic concentrations in the host.

As used herein, the term “biodegradable” means that the materials degrades or breaks down into its component subunits, or digestion, e.g., by a biochemical process, of the material into smaller (e.g., non-polymeric) subunits.

As used herein, the term “microparticles” generally refers to a particle having a diameter less than about 1000 microns. The particles can have any shape.

As used herein, the term “nanoparticle” generally refers to a particle having a diameter from about 10 nm up to, but not including, about 1 micron, or from 100 nm to about 1 micron. The particles can have any shape. The particles can be cubic, for example. Other non-limiting shapes which are contemplated can include tetrahedral, bipyramidal, octahedral, icosahedral, and decahedral shapes.

A composition containing microparticles and/or nanoparticles may include particles of a range of particle sizes. In certain embodiments, the particle size distribution may be uniform, e.g., within less than about a 20% standard deviation of the mean volume diameter, and in other embodiments, still more uniform, e.g., within about 10% of the median volume diameter.

As used herein, the phrase “mean particle size” generally refers to the statistical mean particle size (diameter) of the particles in a population of particles. The diameter of an essentially spherical particle may refer to the physical or hydrodynamic diameter. The diameter of a non-spherical particle may refer to the hydrodynamic diameter. As used herein, the diameter of a non-spherical particle may refer to the largest linear distance between two points on the surface of the particle. Mean particle size can be measured using methods known in the art, such as dynamic light scattering or electronic microscopy such as scanning electron microscopy (SEM) or transmission electron microscopy (TEM).

As used herein, the phrases “monodisperse” and “homogeneous size distribution” are used interchangeably and describe a population of nanoparticles or microparticles where all of the particles are the same or nearly the same size. As used herein, a monodisperse distribution refers to particle distributions in which 90% of the distribution lies within 15% of the median particle size, or within 10% of the median particle size, or within 5% of the median particle size.

As used herein, the phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the phrase “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the halides, hydroxides, carbonates, and bicarbonates of ammonia, sodium, lithium, potassium, cesium, calcium, magnesium, aluminum, and zinc. Salts may also be formed with suitable organic bases, including those that are non-toxic and strong enough to form such salts.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “therapeutically effective amount” refers to an amount of a therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish the symptoms of one or more diseases or disorders, such as reducing tumor size (e.g., tumor volume).

As used herein, the term “about” is intended to describe values either above or below the stated value in a range of approx. +/−10%. The ranges are intended to be made clear by context, and no further limitation is implied. The use of any and all examples, or exemplary language (e.g., “such as”) provided herein, is intended merely to better illuminate the description and does not pose a limitation on the scope of the description unless otherwise claimed.

As used herein, the term “PD-1 antagonist” means any molecule that attenuates inhibitory signal transduction mediated by PD-1, found on the surface of T cells, B cells, natural killer (NK) cells, monocytes, dendritic cells (DC), and/or macrophages. Such an antagonist includes a molecule that disrupts any inhibitory signal generated by a PD-1 molecule on a T cell. Therefore, PD-1 antagonist can be a molecule that inhibits, reduces, abolishes or otherwise reduces inhibitory signal transduction through the PD-1 receptor signaling pathway. Such decrease may result where: (i) the PD-1 antagonist binds to a PD-1 receptor without triggering signal transduction, to reduce or block inhibitory signal transduction; (ii) the PD-1 antagonist binds to a ligand (e.g. an agonist) of the PD-1 receptor, preventing its binding thereto (for example, where said agonist is B7-H1); (iii) the PD-1 antagonist binds to, or otherwise inhibits the activity of, a molecule that is part of a regulatory chain that, when not inhibited, has the result of stimulating or otherwise facilitating PD-1 inhibitory signal transduction; or (iv) the PD-1 antagonist inhibits expression of a PD-1 receptor or expression ligand thereof, especially by reducing or abolishing expression of one or more genes encoding PD-1 or one or more of its natural ligands. Thus, a PD-1 antagonist can be a molecule that affects a decrease in PD-1 inhibitory signal transduction, thereby increasing T cell response to one or more antigens.

As used herein, “CTLA4 antagonist” means a compound that reduces CTLA4-mediated inhibition of T cell reactions. For example, in an T cell, CTLA4 delivers an inhibitory impulse upon binding of B7 ligands, such B7-1 and B7-2. A CTLA4 antagonist is one that disrupts binding of said ligands to CTLA4 on activated T cells.

### II. Compositions

Mammalian cells sustain low ratios of intracellular to extracellular sodium and chloride, and high ratios of potassium (Milo et al., Cell biology by the numbers. pp. xlii, 356 pages). These asymmetric ionic gradients are important to cell functions (Pedersen, et al., J. Am. Soc. Nephrol., 22, 1587 (2011)), driving needed cellular processes including the transport of amino acids, maintenance of cellular pH, and control of cell volume (Okada, Cell Biochem. Biophys. 41, 233-258 (2004), Hoffmann and Pedersen, Acta Physiol 202, 465-485 (2011)). Lowering the extracellular concentrations of sodium and chloride, for instance by immersing cells in a hypotonic solution, causes cytoskeleton destruction, cell cycle arrest, and cell lysis (Galvez et al., Cell Tissue Res 304, 279-285 (2001)). Elevating intracellular osmolarity may induce similar effects, but it is difficult to achieve because ion transport is tightly regulated by live cells.

Over the years, a myriad of inorganic nanoparticles have been prepared and their behaviors inside cells and animals investigated. Yet, some common electrolytes, for instance NaCl, have been left out of this campaign. The underlying assumption is that electrolyte nanoparticles would quickly dissolve in water and behave no different from their consistent salts. However, the examples below show that salt particles, such as NaCl nanoparticles, kill cancer cells to a much greater degree than healthy, non-cancer cells.

A. Salt Particles

1. Core Composition

Particles formed of a salt formed from an alkai metal or alkaline earth metal and halide, also referred to salt particles, and methods of use thereof are provided. These include, for instance, particles of salts which may be formed from alkali metal ions, such as lithium, sodium, potassium, rubidium, and cesium, and halide counterions, such as fluoride, chloride, bromide, and iodide. In some other instances, particles of salts may be formed from alkaline earth metal ions, such as magnesium and calcium, and halide counterions, such as fluoride, chloride, bromide, and iodide. For example, sodium-based salt particles can include sodium chloride particles, sodium fluoride particles, sodium bromide particles, sodium iodide particles, and combinations thereof. Chloride based-particles include sodium chloride particles, potassium chloride (KCl) particles, and calcium chloride (CaCl₂) particles. In some instances, the electrolyte nano- or micro-particles are formed of a single type of salt particle (i.e., sodium chloride), such as those named herein. In other instances, the electrolyte nano- or micro-particles are formed of any combination of different types of salt particles (i.e., sodium chloride and potassium chloride particles), such as those named herein.

In preferred embodiments, the salt particles are NaCl particles, preferably NaCl nanoparticles. Although the compositions and methods described in detail herein focus primarily upon NaCl particles, particularly NaCl nanoparticles, corresponding embodiments of particles formed from other salts formed from an alkai metal or alkaline earth metal and halide such as those provided above are also specifically disclosed and can substitute for, or supplement, NaCl particles in the compositions and methods provided herein.

Salt particles, particularly NaCl nanoparticles, can be exploited as a Trojan-horse strategy to deliver ions into cells to disturb the ion homeostasis. Each NaCl nanoparticle contains with it millions of sodium and chlorine atoms, but they are not checked at the ion pumps or channels for cell entry (Gadsby, Nat. Rev. Mol. Cell Biol., 10, 344 (2009). Yu and Catterall, Genome Biol. 4, 207 (2003)). Instead, NaCl nanoparticles enter cells through endocytosis, which potentially allows them to bypass cell regulations on ions. Due to high water solubility of NaCl, these nanoparticles quickly degrade inside cells, releasing large quantities of Na+ and Cl−. Limited by intrinsic osmotic gradients, these ions are not able to freely move across the plasma membrane, amounting to an osmotic shock that extensively perturbs cell functions.

The studies provided below show that NaCl nanoparticles but not salts can effectively kill cancer cells. This is because the nanoparticles enter cells through endocytosis, bypassing cell regulations on ion transport; when dissolving inside cells, the released ions amount to a surge of osmolarity, causing pyroptosis, a programmed necrosis mechanism. Normal cells are highly resistant to the treatment, a phenomenon believed to be largely due to their intrinsically low Na+ levels relative to cancer cells. In vivo studies confirm NaCl nanoparticles can be used for cancer treatment.

The disclosed particles are typically nanoscale in size, for example, having a diameter of 10 nm up to, but not including, about 1 micron. However, it will be appreciated that in some embodiments, and for some uses, the particles can be smaller or larger (e.g., microparticles, etc.). Although many of the compositions disclosed herein are referred to as nanoparticle compositions, it will be appreciated that in some embodiments and for some uses the carrier can be somewhat larger than nanoparticles. For example, carrier compositions can also include particles having a diameter of between about 1 micron to about 1000 microns. Such compositions can be referred to as microparticle compositions.

Nanoparticles are often utilized for intertissue applications and penetration of cells. Thus, in some embodiments, the particles are nanoparticles that have any diameter from 10 nm up to about 1,000 nm. For example, the nanoparticles can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm, from 10 nm to 100 nm. For example, in some embodiments, the particles are about 15 nm, 25 nm, 60 nm, 100 nm, or any other integer value or range of values between 1 nm and 1000 nm inclusive. In some embodiments the nanoparticles can have a diameter less than 400 nm, less than 300 nm, or less than 200 nm. For example, the nanoparticle can have a diameter from between 50 nm and 300 nm.

In one example, the average diameters of the nanoparticles are between about 15 nm and about 800 nm, or about 50 nm and about 500 nm, or between about 50 nm and about 350 nm. In some embodiments, the average diameters of the nanoparticles are about 100 nm.

In some embodiments for treating cancer it is desirable that the particle be of a size suitable to access the tumor microenvironment. In particular embodiments, the particle is of a size suitable to access the tumor microenvironment and/or the tumor cells by enhanced permeability and retention (EPR) effect. EPR refers to the property by which certain sizes of molecules tend to accumulate in tumor tissue much more than they do in normal tissues. Therefore, in an exemplary composition for treatment of cancer, the delivery vehicle can be in the range of about 25 nm to about 500 nm. In another example, the delivery vehicle can be in the range of about 50 nm to about 300 nm inclusive. In another example, the delivery vehicle can be in the range of about 80 nm to about 120 nm inclusive. In another example, the delivery vehicle can be in the range of about 85 nm to about 110 nm inclusive.

Preferably the particles of a size that can be internalized by cancer cells by endocytosis.

Particles size can be measure or determined by, for example, dynamic light scattering, electronic microscopy such as scanning electron microscopy (SEM), and transmission electron microscopy (TEM).

In some embodiments the salt particles in a particle compositions are monodispersed. In some embodiments, the salt particles in a particle composition are of various sizes (i.e., polydispersed).

2. Methods of Making Salt Particles

The disclosed salt particles are preferably formed of sodium and chloride, though other salts such as those mentioned above are also specifically contemplated.

The particles are typically formed in organic solvents using appropriate sodium and chloride precursors. In some embodiments, sodium oleate and molybdenum chloride are utilized as sodium and chloride precursors. The particles can be synthesized through a reaction using, for example, hexane/ethanol mixed solvent and oleylamine surfactant. NaCl nanoparticles can also be referred to as sodium chloride nanoparticles and SCNPs.

The microemulsion reaction can include, for example, adding molybdenum (V) chloride to a solvent solution including a solvent, a reductant, a surfactant, and sodium oleate. In particular embodiments, the solvent is a mixture of hexane and ethanol. The reductant is hexadecanediol or tetradecanediol and the surfactant is oleylamine or oleic acid. Although referred to herein as microemulsion reactions, such reactions can be, and preferably are, free from water.

In an exemplary method of making SCNPs, sodium oleate, oleylamine, and 1,2-tetradecanediol are dissolved in a solvent solution, for example a mixed solution such as hexane/ethanol. Molybdenum (V) chloride is added and mixed with the solution (e.g., 24 hours at 60 degrees C.). The raw products are collected by centrifugation (e.g., 12000 RPM for 10 min). The particles are redispersed in a suitable solution, for example hexane, with brief sonication followed by centrifugation. The particles can be collected and redispersed repeatedly to reduce the presence of unreacted precursors.

Such a reaction can yield cubic phase hydrophobic NaCl nanoparticles having a sodium and chloride molar ratio of about 1:1. The particles formed according to this method have a narrow size distribution and negligible impurities including, for example, molybdenum. The size is tunable from 10 to 1000 nm by changing the reaction conditions, such as the ratio between the sodium/chloride precursors and oleylamine, the reaction volume, temperature, and stirring speed (e.g., magnetic stirring speed).

Alternatively, NaCl nanoparticles can be synthesized by a coprecipitation method using Sodium Acetate or Sodium Oleate or another sodium fatty acid salt, and Acetyl Chloride as precusors and Ethanol as solvent. Fatty acid salts include, but are not limited to sodium salts of myristic, oleic, palmitic, stearic, acids or mixtures thereof.

For example, in an exemplary protocol, 140 mg Sodium Acetate is dissolved 20 mL Ethanol at room temperature. 120 μL Acetyl Chloride is added in to the mixture to react for 10 min. The white raw products are collected by centrifugation (e.g., 12000 RPM for 10 min). The particles are redispersed in a suitable solution, for example ethanol, with brief sonication followed by centrifugation. The particles can be collected and redispersed repeatedly to reduce the presence of unreacted precursors.

The above-mentioned reactions can be extended to synthesizing other electrolyte nano- or micro-particles discussed herein. For example, the method of making NaCl nanoparticle described herein can be adapted to make KCl nanoparticles in the similar size range with potassium oleate as the precursor. For the reagents, surfactant are all the same as NaCl nanoparticle synthesis method described above.

B. Coating

The particles can include a coating. For example, NaCl nanoparticles synthesized as described above can be hydrophobic because of the oleylamine or oleic acid coating. A hydrophilic layer added to nanoparticles made them more compatible with aqueous solutions. In some cases, for NaCl nanoparticles made from co-precipitation using sodium acetate, the coating is less hydrophobic. Still, an additional coating can be added to extend the half-lives of the nanocrystals in water and/or improve nanoparticle uptake by cells. Thus, in some embodiments, the disclosed particles have coating a hydrophilic coating or exterior.

1. Composition of the Coating

The coating can be composed of, for example, amphiphilic block co-polymers, peptides, proteins, lipids, or combinations thereof. In some embodiments, the coating is composed of conjugates or fusions of two or more of the foregoing alone or in further combination with one or more active agents.

a. Lipids

The coating can be, or include, one or more lipids. Lipids and other components useful in preparing the disclosed nanoparticle compositions having a lipid-based coating are known in the art. Suitable neutral, cationic and anionic lipids include, but are not limited to, sterols and lipids such as cholesterol, phospholipids, lysolipids, lysophospholipids, and sphingolipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as I-ceramidyl glucosides) such as ceramide galactopyranoside, gangliosides and cerebrosides; fatty acids, sterols, containing a carboxylic acid group for example, cholesterol; phosphoethanolamines such as 1,2-distearoyl-sn-glycero-3-phosphoethanolamine (DSPE), 1,2-diacyl-sn-glycero-3-phosphoethanolamine, including, but not limited to, 1,2-dioleylphosphoethanolamine (DOPE), 1,2-dihexadecylphosphoethanolamine (DHPE); and phophatidylcholines such as 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

The lipid can be a sphingomyelin metabolites such as, without limitation, ceramide, sphingosine, or sphingosine 1-phosphate.

Exemplary catonic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes. N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-urimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids can be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine).

A sterol component may be included to confer a physicochemical and biological behavior. Such a sterol component may be selected from cholesterol or its derivative e.g., ergosterol or cholesterolhemisuccinate.

The coating can include a single type of lipid, or a combination of two or more lipids.

b. Polyethers and Polyquaterniums

The coating can be, or include, a polyether. Exemplary polyethers include, but are not limited to, oligomers and polymers of ethylene oxide. In preferred embodiments, the polyether is a Polyethylene glycol (PEG). PEGs are prepared by polymerization of ethylene oxide and are commercially available over a wide range of molecular weights from 300 g/mol to 10,000,000 g/mol, and can have branched, star, or comb geometries. The numbers that are often included in the names of PEGs indicate their average molecular weights (e.g. a PEG with n=9 would have an average molecular weight of approximately 400 daltons, and would be labeled PEG 400.) Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). Mw and Mn can be measured by mass spectrometry. In some embodiment the PEG is an amino(polyethylene glycol) (also referred to as a PEG amine).

In some embodiments, the PEG or PEG amine is up about 25,000, or more. In some embodiments, the PEG or PEG amine is about PEG 350 to about PEG 25.000, or about PEG 350 to about PEG 20,000. In some embodiments, the PEG or PEG amine is about PEG 350 to about PEG 5000, or between about PEG 750 and about PEG 5000, or between about PEG 1000 and PEG 3000. In a particular embodiment, the PEG is PEG 2000.

In particular embodiments, the coating is a polyether-lipid (e.g., phospholipid) conjugate coating. In some embodiments, the polyether-phospholipid conjugate is DSPE-PEG2000 amine. See, for example, the experiments below which describe coating DSPE-PEG2000 amine, onto the nanoparticle surface.

In some embodiments, the coating includes or is formed of one or more polyquaterniums. Polyquaternium is the International Nomenclature for Cosmetic Ingredients designation for several polycationic polymers that are used in the personal care industry. Polyquaternium is a neologism used to emphasize the presence of quaternary ammonium centers in the polymer. INCI has approved at least 40 different polymers under the polyquaternium designation. Different polymers are distinguished by the numerical value that follows the word “polyquaternium”, and include, e.g., polyquaternium-1 through polyquaternium-20, polyquaternium-22, polyquaternium-24, polyquaternium-27 through polyquaternium-37, polyquaternium-39, and polyquaternium-42 through polyquaternium-47. In particular embodiments, the polyquaternium is polyquaternium-7, -10, or -30.

c. Amphiphilic block co-polymers

In some embodiments, the hydrophilic layer or coating around the salt particles is formed of amphiphilic block co-polymers. Polymer refers to a molecular structure including one or more repeat units (monomers), connected by covalent bonds. A biocompatible polymer refers to a polymer that does not typically induce an adverse response when inserted or injected into a living subject. A copolymer refers to a polymer formed of two or more different monomers. The different units may be arranged in a random order, in an alternating order, or as a “block” copolymer, i.e., including one or more regions each including a first repeat unit (e.g., a first monomer or block of monomers), and one or more regions each including a second repeat unit (e.g., a second block), etc. Block copolymers may have two (a diblock copolymer), three (a triblock copolymer), or more numbers of distinct blocks.

The term “amphiphilic” refers to a molecule that has both a polar portion and a non-polar portion. In some embodiments, the polar portion (e.g., a hydrophilic portion such as a hydrophilic polymer) is soluble in water, while the non-polar portion (e.g., a hydrophobic portion such as a hydrophobic polymer) is insoluble in water. The polar portion may have either a formal positive charge, or a formal negative charge. Alternatively, the polar portion may have both a formal positive and a negative charge, and be a zwitterion or inner salt.

The hydrophilic portion of the amphiphilic material can form a corona around the salt particle that increases the salt particle's solubility in aqueous solution. In a particular embodiment, the amphiphilic material is a hydrophobic, biodegradable polymer terminated with a hydrophilic block.

The hydrophilic portion and hydrophobic portion can be biocompatible hydrophilic and hydrophobic polymers respectively. Exemplary biocompatible polymers include, but are not limited to, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polylactides, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt; polyacrylic acid polymers such as polymers of acrylic and methacrylic esters such as poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyalkylenes such as polyethylene, polypropylene poly(ethylene glycol), poly(ethylene oxide), and poly(ethylene terephthalate), poly(vinyl alcohols), poly(vinyl acetate), poly vinyl chloride polystyrene and polyvinylpryrrolidone, derivatives thereof, linear and branched copolymers and block copolymers thereof, and blends thereof.

Other exemplary biodegradable polymers include, but are not limited to, polyesters, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the co-polymer include one or more biodegradable hydrophobic polyesters such as poly(lactic acid), poly(glycolic acid), and poly(lactic-co-glycolic acid), and/or these polymers conjugated to polyalkylene oxides such as polyethylene glycol or block copolymers such as the polypropylene oxide-polyethylene oxide PLURONICs®.

The molecular weight of the biodegradable oligomeric or polymeric segment or polymer can be varied to tailor the properties of the polymer.

In some embodiments, the hydrophilic polymers or segment(s) or block(s) include, but are not limited to, homo polymers or copolymers of polyalkene glycols, such as poly(ethylene glycol), poly(propylene glycol), poly(butylene glycol), and acrylates and acrylamides, such as hydroxyethyl methacrylate and hydroxypropyl-methacrylamide.

The hydrophobic portion of amphiphilic materials can provide a non-polar polymer matrix coating for loading non-polar drugs.

2. Active Agents

The disclosed salt particles can have a molecular and even therapeutic effect without any additional active agent, and thus in some embodiments, the salt particles alone are the active material and the particles do not include (i.e., are free from) an additional active agent. Alternatively, the particle can optionally include one or more active agent. For example, in some embodiments, the hydrophilic layer or coating is, or includes an active agent. In some embodiments, the active agent or agents are conjugated to a component of the hydrophilic layer or otherwise attached to the surface of the layer, or incorporated, loaded or encapsulated into the layer itself. In some such embodiments, the salt core of the particles remains free of additional active agents.

In an exemplary embodiment, the coating includes lipids and the active agent or agent(s) are loaded or otherwise incorporated into or beneath the lipid layer, for example by adding the active agent to the reaction mixture when the lipid components are added to the surface of the salt particles.

The active agent or agents can be, for example, nucleic acids, proteins, and/or small molecules. Exemplary active agents include, for example, tumor antigens, CD4+ T-cell epitopes, cytokines, chemotherapeutic agents, radionuclides, small molecule signal transduction inhibitors, photothermal antennas, immunologic danger signaling molecules, other immunotherapeutics, enzymes, antibiotics, antivirals, anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories, immunomodulators (including ligands that bind to Toll-Like Receptors (including but not limited to CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the delivery vehicle into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, and triplex forming agents).

3. Methods of Making a Coating

In an exemplary coating method, SCNPs in solvent (e.g., hexane) are sonicated and mixed with phospholipid solution (e.g., DSPE-PEG (2000) Amine (1,2-distearoyl-sn-glycero-3-phosphoethanolamine-N-[amino(polyethylene glycol)-2000] (ammonium salt). The solvent can be removed (e.g., under reduced pressure (e.g., at 40° C. using a Rotavapor)). PBS, water, or another suitable aqueous carrier can be added and mixed (e.g., sonicated) to resuspend the particles.

The resulting, phospholipid coated NaCl nanoparticles (also referred to as PSCNPs) were well dispersed in aqueous solutions, bore a hydrodynamic size of 98.0±13.1 nm, and a positive surface charge of +9.7 mV.

The phospholipid coating also rendered NaCl nanocrystals with extended lifetimes in water but does not stop the degradation process. Indeed, TEM analysis found small cavities on the nanocrystal surface when PSCNPs were incubated in water for 1-2 h. Further incubation led to significant particle disintegration and eventually complete dissolution. Preferably, the coated nanoparticles described have extended lifetimes of between about 1 and 48 hours, about 1 and 24 hours, about 1 and 12 hours. In some instances, the coated nanoparticles described have extended lifetimes of at least about 0.5, 1, 2, 3, 4, 5, 6, 7, 8, 9, 10, 11, 12, 13, 14, 15, 16, 17, 18, 19, 20, 21, 22, 23, 24, 30, 35, 40, 48 hours, or longer.

The particle coatings described can impart a surface charge on the coated salt particles. In some instances, the coated particles have a zeta potential of between about −60 mV and about +60 mV, −50 mV and about +50 mV, −40 mV and about +40 mV, −30 mV and about +30 mV, between about −20 mV and about +20 mV, between about −10 mV and about +10 mV, or between about −5 mV and about +5 mV. In some instances, the zeta potential of the coated particles is about +5, +6, +7, +8, +9, +10, +11, +12, +13, +14, or +15 mV.

### III. Formulations

Pharmaceutical compositions including the disclosed salt particles, for example NaCl nanoparticles, are provided. Pharmaceutical compositions can be for, for example, administration by parenteral (e.g., intramuscular, intraperitoneal, intravenous (IV) or subcutaneous) injection.

In some embodiments, the compositions are administered systemically, for example, by intravenous or intraperitoneal administration, in an amount effective for delivery of the compositions to targeted cells.

In certain embodiments, the compositions are administered locally, for example, by subcutaneous injection, or injection directly into a site to be treated. In some embodiments, the compositions are injected or otherwise administered directly to one or more tumors. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the particles to the immediate area of the implant.

In some embodiments, the particle compositions are intravesically administered to the bladder. Such a method of delivery is particularly useful for the treat bladder cancer.

The salt particles, for example NaCl nanoparticles, can be provided to the cell either directly, such as by contacting it with the cell, or indirectly, such as through the action of any biological process. For example, the salt particles, for example NaCl nanoparticles, can be formulated in a physiologically acceptable carrier or vehicle, and injected into a tissue or fluid surrounding the cell.

A. Formulations for Parenteral Administration

In a preferred embodiment the compositions are administered in an aqueous solution, by parenteral injection.

The formulation can be in the form of a suspension or emulsion. In general, pharmaceutical compositions are provided including effective amounts of salt particles, for example NaCl nanoparticles, optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions., or by heating the compositions.

In some embodiments, increasing temperature of a colloidal solution of salt particles is avoided. In some embodiments, lipid salt nanoparticles can be prepared in a thin film, which can optionally undergo heating. For example, phospholipid can be mixed with nanoparticles in organic solvents such as chloroform. After evaporating chloroform, a thin film is left on the vessel interior surface. Nanoparticles can be shipped in this manner. Before treatment, water/buffer solutions are added to the vessel to redisperse nanoparticles in aqueous solutions.

B. Other Formulations

The salt particles, for example NaCl nanoparticles, can also be applied topically. Topical administration can include application to the lungs, nasal, oral (sublingual, buccal), vaginal, or rectal mucosa. These methods of administration can be made effective by formulating the salt particles, for example NaCl nanoparticles, with transdermal or mucosal transport elements.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa can be incorporated into a tablet, gel, capsule, suspension or emulsion. Standard pharmaceutical excipients are available from any formulator.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29(6):703-8 (2008).

Transdermal formulations may also be prepared. These will typically be ointments, lotions, sprays, or patches, all of which can be prepared using standard technology. Transdermal formulations can include penetration enhancers.

### IV. Methods of Use

A. Methods of Treatment

The particle compositions can be used to treat diseases and disorders including cancer in vivo. A typical in vivo method includes administering to a subject in need thereof an effective amount of salt particles, for example NaCl nanoparticles, to reduce one or more symptoms of the disease or disorder.

The disclosed compositions and methods of treatment thereof are particularly useful in the context of cancer, including tumor therapy. Accordingly, methods of treating cancer are provided.

In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors can exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The disclosed compositions and methods can be used to treat both benign and malignant tumors. The disclosed methods typically include administering a subject in need there of an effective amount to the composition to reduce one or more symptoms, or molecular, or physiological indicators of the tumors or cancer. For example, therapeutically effective amounts of the disclosed compositions used in the treatment of cancer will generally kill tumor cells or inhibit proliferation or metastasis of the tumor cells or a combination thereof. The compositions and methods are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

Symptoms of cancer may be physical, such as tumor burden, or biological such as apoptosis or necrosis of tumor cells. For example, the composition can be administered in an amount effective to kill cancer cells, improve survival of a subject with cancer, or a combination thereof. In some embodiments, the amount is effective to reduce mitochondrial oxygen consumption rate (OCR), reduce mitochondrial respiration rate (MSR), decrease intracellular ATP level, increase the ROS level, increase levels of JNK, ERK, and/or p38 phosphorylation, increase lipid peroxidation, increase DNA damage, release of cytochrome c, increase of caspase-3 activity, increase caspase-1 activity, increase cell swelling and/or bleb formation, induce cell rupture and/or complete osmotic lysis, increase NLRP3 inflammasome induction, increase GSDMD N-terminal fragment release, elevate IL-1β secretion, increase intracellular K⁺ level, increased presentation/secretion of calreticulin (CRT), increased presentation/secretion of adenosine triphosphate (ATP), h increased presentation/secretion of high mobility group box 1 (HMGB1), or a combination thereof in tumor and/or cancer cells. Preferably, the composition is administered in an amount or/manner that the foregoing are altered or effected to a greater degree in tumor and/or cancer cells than non-tumor or non-cancer (e.g., control, or health) cells.

In some embodiments, the amount is effective to increase apoptosis, necrosis, and or pyroptosis of tumor and/or cancer cells. Preferably, the composition is administered in an amount or/manner that the foregoing are increased to a greater degree in tumor and/or cancer cells than non-tumor or non-cancer (e.g., control, or health) cells.

In some embodiments, the tumor and/or cancer cells have a higher [Na⁺]ᵢₙₜ than non-tumor or non-cancer (e.g., control, or health) cells.

The actual effective amounts of composition can vary according to factors including the specific, the particular composition formulated, the mode of administration, and the age, weight, condition of the subject being treated, as well as the route of administration and the disease or disorder.

An effective amount of the composition can be compared to a control. Suitable controls are known in the art. A typical control is a comparison of a condition or symptom of a subject prior to and after administration of the composition. The condition or symptom can be a biochemical, molecular, physiological, or pathological readout. In another embodiment, the control is a matched subject that is administered a different therapeutic agent. Accordingly, the compositions disclosed here can be compared to other art recognized treatments for the disease or condition to be treated.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

In some embodiments, the salt particles, for example NaCl nanoparticles, are administered to a subject in need thereof at a dosage of about 0.1 mg/kg to about 1,000 mg/kg, or about 1 mg/kg to about 100 mg/kg, or about 5 mg/kg to about 50 mg/kg, or any integer mg/kg between 1 and 1,000 inclusive.

In the working Examples below, the NaCl nanoparticles were administered in a mouse tumor model at dosages 50 μL of 9-30 mg/mL NaCl nanoparticle solution per mouse weighing between about 15 g and 30 g. Thus, in the illustrative tumor model, a dosage of about 15-100 mg/kg was therapeutically effective.

B. Vaccination

One appealing property of NaCl nanoparticles in the context of cancer therapy is that they induce immunogenic cell death (TCD). While the majority of chemotherapeutics induce non-immunogenic or tolerogenic cell death, a small fraction of them stimulate an immune response when killing cancer cells, and necrosis is an immunogenic process (Inoue and Tani, Cell Death Differ., 21, 39 (2014), Zhang, et al., Cell Res., 28, 9 (2018)). Recent studies show that ICD comes from these selected drugs' ability to promote the expression/secretion of certain damage-associated molecular pattern (DAMP) molecules, most importantly CRT. ATP, and HMGB1. These ICD signals communicate a state of danger to the organism, promoting the recruitment of professional antigen-presenting cells (APCs), mostly importantly dendritic cells (DCs), to tumors. The ICD signals also facilitate the activation and antigen cross-presentation by DCs, and as a result elicit antigen-specific immunity. In other words, ICD produces an in situ vaccine that promotes selective, immune-mediated cancer cell eradication.

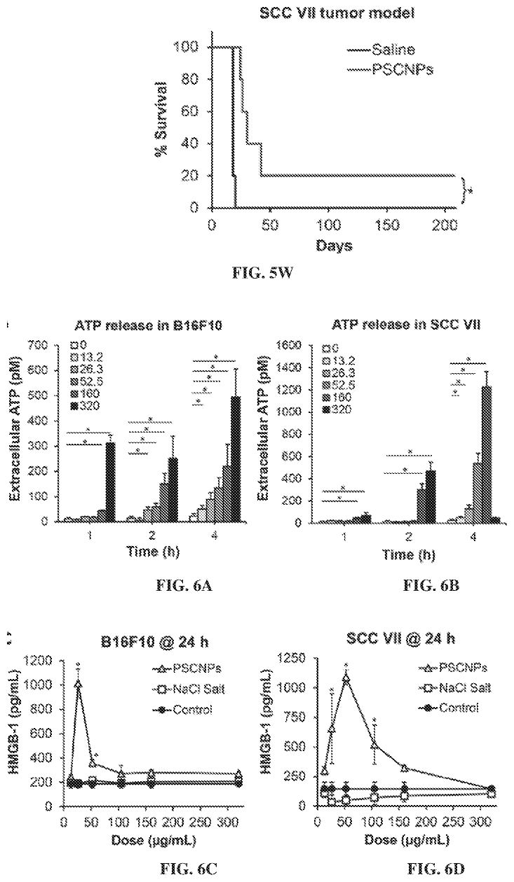

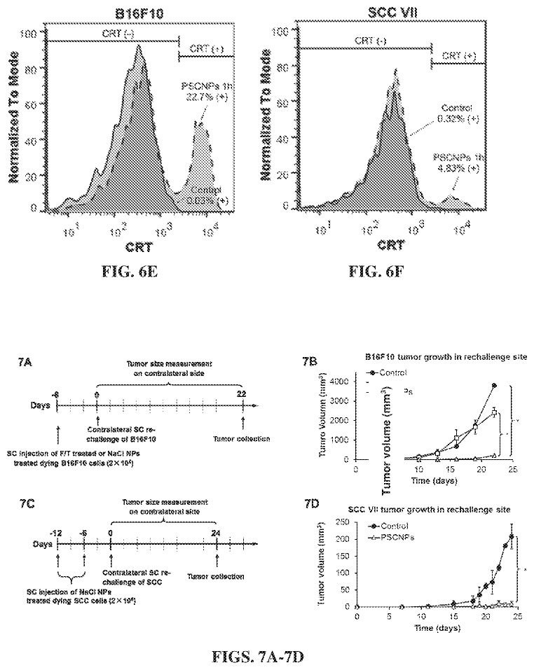

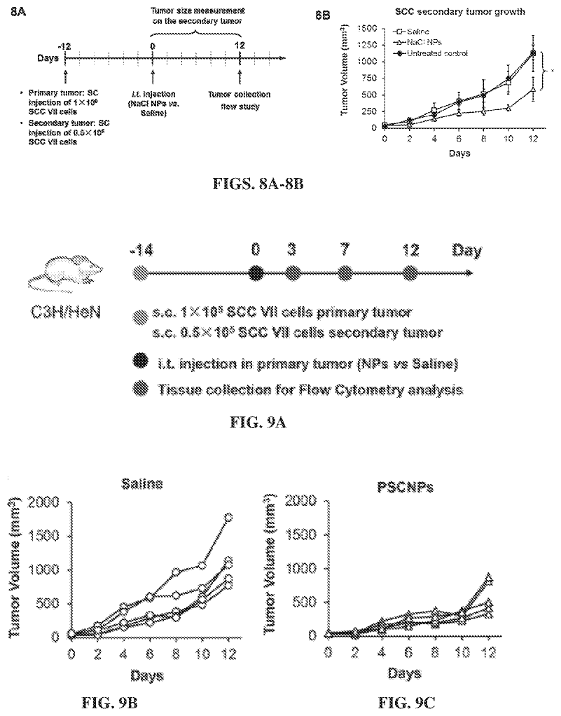

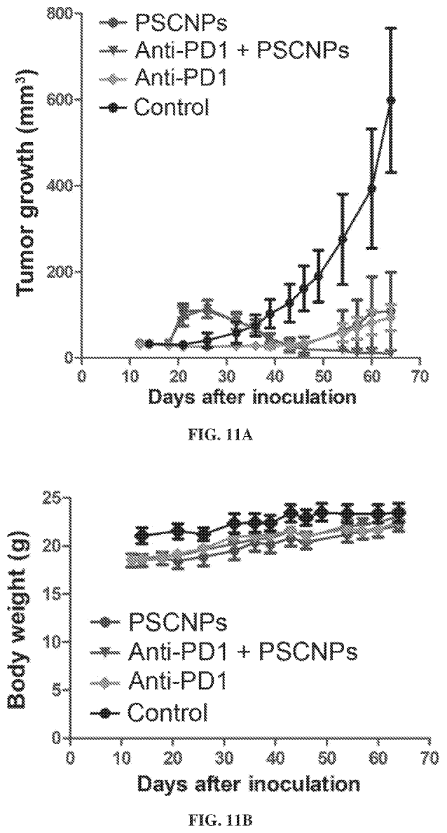

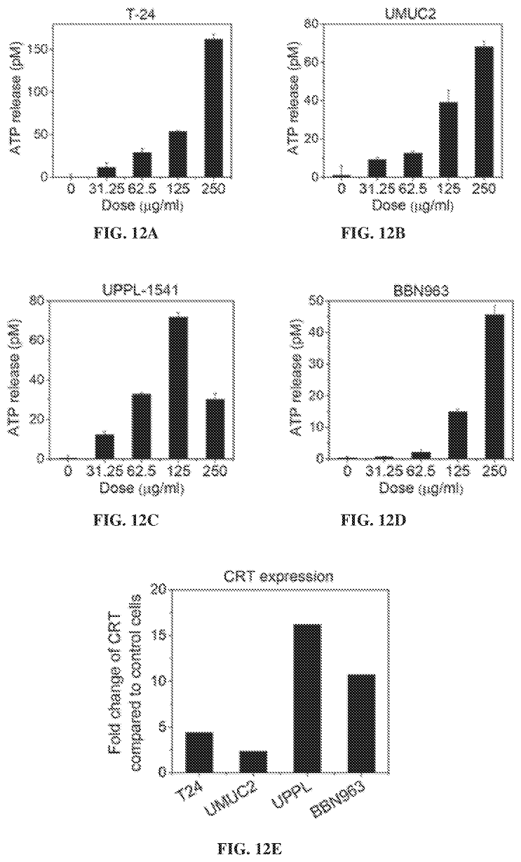

The experimental examples below illustrate that NaCl nanoparticles are a powerful ICD agent. Cancer cells succumbing to NaCl nanoparticles are associated with elevated ATP, HMGB1, and CRT presentation/secretion (FIGS. 6A-6E, FIGS. 12A-12E). Moreover, cancer cells killed by NaCl nanoparticles were subcutaneously injected into immunocompetent mice, and the vaccination protected the mice against a subsequent challenge with live tumor cells (FIG. 7A-7D & Table 3). When NaCl nanoparticles were injected directly into tumors the treatment promoted anticancer immunity which slowed down the growth of a secondary tumor inoculated to the opposite flank (FIGS. 8A-8B & Table 4). All these results indicate that in addition to directly killing cancer cells, NaCl nanoparticles can also stimulate anticancer immunity that helps tumor control at both local and distant sites.

Thus, the salt particles, for example NaCl nanoparticles, described herein can be administered as a component of a vaccine. Vaccines disclosed herein can include salt particles, for example NaCl nanoparticles, alone and optionally antigens and/or adjuvants. Additionally or alternatively, the vaccines can include particle-induced antigens alone or in combination with particles. For example, in some embodiments, the antigens are derived from cancer cells in the subject that die following administration of the particles, preferably sodium chloride nanoparticles. Thus, no additional antigen need be administered. In other embodiments, antigens and/or adjuvants are administered to the subject in need thereof.

In some embodiments, the antigens are derived from cancer cells in vitro or ex vivo. The cancer cells can be cancer cells that were induced to die, by, for example, apoptosis, necrosis, or another mechanism. For example, in some embodiments, the cells are contacted in vitro or ex vivo with an effective amount to salt particles, for example NaCl nanoparticles, to induce cell death. The dead and/or dying cancer cells or a lysate, extract, fraction, isolate, or secreted factors thereof can be administered to a subject in need there as antigen. The cancer cells or cell-derived antigen can be administered to the subject alone or in combination with particles and/or an additional adjuvant. In preferred embodiments, the dead and/or dying cancer cells were contacted with an effective amount of salt particles, for example NaCl nanoparticles, to elevate ATP, HMGB1, and/or calreticulin (CRT) presentation/secretion.

The cancer cells can be isolated from the subject to be treated (e.g., personalized medicine), or another subject, or can be from a cell line or other source. In some embodiments, the isolated cells are cultured and/or propagated in vitro or ex vivo prior to treatment with particles.

1. Antigens

Antigens can be peptides, proteins, polysaccharides, saccharides, lipids, nucleic acids, or combinations thereof. The antigen can be derived from a transformed cell such as a cancer or leukemic cell and can be a whole cell or immunogenic component thereof. Suitable antigens are known in the art and are available from commercial government and scientific sources. The antigens can be purified or partially purified polypeptides derived from tumors or can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein. PTPRK, K-ras, N-ras, Triosephosphate isomerase, Bage-1, Gage 3,4,5,6,7, GnTV, Herv-K-mel, Lage-1, Mage-A1,2,3,4,6,10,12. Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

In some embodiments the antigen is a neoantigen or a patient-specific antigen. Recent technological improvements have made it possible to identify the immune response to patient-specific neoantigens that arise as a consequence of tumor-specific mutations, and emerging data indicate that recognition of such neoantigens is a major factor in the activity of clinical immunotherapies (Schumacher and Schreidber, Science, 348(6230):69-74 (2015). Neoantigen load provides an avenue to selectively enhance T cell reactivity against this class of antigens.

Traditionally, cancer vaccines have targeted tumor-associated antigens (TAAs) which can be expressed not only on tumor cells but in the normal tissues (Ito, et al., Cancer Neoantigens: A Promising Source of Immunogens for Cancer Immunotherapy. J Clin Cell Immunol, 6:322 (2015) doi:10.4172/2155-9899.1000322). TAAs include cancer-testis antigens and differentiation antigens, and even though self-antigens have the benefit of being useful for diverse patients, expanded T cells with the high-affinity TCR (T-cell receptor) needed to overcome the central and peripheral tolerance of the host, which would impair anti-tumor T-cell activities and increase risks of autoimmune reactions.

Thus, in some embodiments, the antigen is recognized as “non-self” by the host immune system, and preferably can bypass central tolerance in the thymus. Examples include pathogen-associated antigens, mutated growth factor receptor, mutated K-ras, or idiotype-derived antigens. Somatic mutations in tumor genes, which usually accumulate tens to hundreds of fold during neoplastic transformation, could occur in protein-coding regions. Whether missense or frameshift, every mutation has the potential to generate tumor-specific antigens. These mutant antigens can be referred to as “cancer neoantigens” Ito, et al., Cancer Neoantigens: A Promising Source of Immunogens for Cancer Immunotherapy. J Clin Cell Immunol, 6:322 (2015) doi:10.4172/2155-9899.1000322. Neoantigen-based cancer vaccines have the potential to induce more robust and specific anti-tumor T-cell responses compared with conventional shared-antigen-targeted vaccines. Recent developments in genomics and bioinformatics, including massively parallel sequencing (MPS) and epitope prediction algorithms, have provided a major breakthrough in identifying and selecting neoantigens.

Methods of identifying, selecting, and validating neoantigens are known in the art. See, for example. Ito, et al., Cancer Neoantigens: A Promising Source of Immunogens for Cancer Immunotherapy. J Clin Cell Immunol, 6:322 (2015) doi:10.4172/2155-9899.1000322, which is specifically incorporated by reference herein in its entirety. For example, as discussed in Ito, et al., a non-limiting example of identifying a neoantigen can include screening, selection, and optionally validation of candidate immunogens. First, the whole genome/exome sequence profile is screened to identify tumor-specific somatic mutations (cancer neoantigens) by MPS of tumor and normal tissues, respectively. Second, computational algorithms are used for predicting the affinity of the mutation-derived peptides with the patient's own HLA and/or TCR. The mutation-derived peptides can serve as antigens for the compositions and methods disclosed herein. Third, synthetic mutated peptides and wild-type peptides can be used to validate the immunogenicity and specificity of the identified antigens by in vitro T-cell assay or in vivo immunization.

2. Adjuvants

Optionally, the vaccines described herein may include adjuvants. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminum salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor. Co-stimulatory molecules, including polypeptides of the B7 family, may be administered. Such proteinaceous adjuvants may be provided as the full-length polypeptide or an active fragment thereof, or in the form of DNA, such as plasmid DNA.

C. Subjects to be Treated

Tumors, for example malignant tumors, which may be treated can be classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, as well as adenocarcinomas and sarcomas.

The cancer can be, for example, bone, bladder, brain, breast, cervical, colo-rectal, esophageal, kidney, liver, lung, nasopharyngeal, pancreatic, prostate, skin, stomach, or uterine cancer.

In some embodiments, the disclosed compositions are used to treat multiple cancer types concurrently. The compositions can also be used to treat metastases or tumors at multiple locations.

The frequency of administration can be, for example, one, two, three, four or more times daily, weekly, every two weeks, or monthly. In some embodiments, the composition is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once, twice or three times weekly, or is once, twice or three times every two weeks, or is once, twice or three times every four weeks. In some embodiments, the composition is administered to a subject with cancer 1-3 times, preferably 2 times, a week.

D. Combination Therapies

Combination therapies are also disclosed. The disclosed compositions can include, or can be administered to a subject in need thereof alone or in combination with one or more additional therapeutic agents. The additional therapeutic agents are selected based on the condition, disorder or disease to be treated. For example, the liposomal-drug composition can be co-administered with one or more additional agents that treat cancer. In a preferred embodiment the additional therapeutic agent targets a different pathway so that the combined effect of the therapies is greater than each alone.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic agents can be administered locally or systemically to the subject, or coated or incorporated onto, or into a device or graft. The additional agent(s) can be part of polymeric nanoparticles, liposomes or another delivery vehicles, or as free-drug.

The different active agents can have the same or different mechanisms of action. In some embodiments, the combination results in an additive effect on the treatment of the disease or disorder. In some embodiments, the combinations result in a more than additive effect on the treatment of the disease or disorder. In particular embodiments, the additional active agent increases or improves or further improves or increases an immune stimulating or immune enhancing response compared to administration of the salt particles, for example NaCl nanoparticles, alone.

Salt particles, for example NaCl nanoparticles, and one or more additional active agents can be administered to a subject as part of a treatment regimen. Treatment regimen typically refers to a treatment of a disease or a method for achieving a desired physiological change or change in a symptom of the disease. For example, in a particle embodiments, the regimen leads to an increased or enhanced response of the immune system to an antigen or immunogen, an increase in the number or activity of one or more cells, or cell types, that are involved in such response, wherein said treatment or method includes administering to an animal, such as a mammal, especially a human being, a sufficient amount of two or more chemical agents or components of the regimen to effectively treat the disease or to produce said physiological change or change in a symptom of the disease, wherein the chemical agents or components are administered together, such as part of the same composition, or administered separately and independently at the same time or at different times (i.e., administration of each agent or component is separated by a finite period of time from one or more of the agents or components). Preferably, administration of the one or more agents or components achieves a result greater than that of any of the agents or components when administered alone or in isolation. Typically one of the agents is particles, preferably sodium chloride nanoparticles.

Salt particles, for example NaCl nanoparticles, and/or additional active agent(s) can be administered together or separately on a daily basis for a finite time period, such as up to 3 days, or up to 5 days, or up to 7 days, or up to 10 days, or up to 15 days or up to 20 days or up to 25 days, are all specifically contemplated. In some embodiments, the particle composition and/or additional active agent(s) is administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once weekly, or is once every two weeks, or is once every four weeks, or is twice every week. In some embodiments, a single administration is effective. In some embodiments two or more administrations are needed.

All such administrations of the salt particle, for example NaCl nanoparticle, composition may occur before or after administration of the additional active agent(s). Alternatively, administration of one or more doses of active agent(s) may be temporally staggered with the administration of a particle composition to form a uniform or non-uniform course of treatment whereby one or more doses of active agent(s) are administered, followed by one or more doses of particle composition, followed by one or more doses of additional active agent(s); or vice versa, all according to whatever schedule is selected or desired by the researcher or clinician administering the agents.

In some embodiments the particle composition is administered at least 1, 2, 3, 5, 10, 15, 20, 24 or 30 minutes, hours, days, or weeks prior to or after administering of the additional active agent(s). In some embodiments, the additional active agent(s) is administered at least 1, 2, 3, 5, 10, 15, 20, 24 or 30 minutes, hours, days, or weeks prior to or after administering of the particle composition.

In some embodiments, the additional active agent is administered in a range of about 0.1 mg/kg to 100 mg/kg, or about 0.1 mg/kg to 1 mg/kg; or about 10 mg/kg to 100 mg/kg; or 0.1-1 mg/kg to 10-100 mg/kg (e.g., daily; or 2, 3, 4, 5 or more times weekly; or 2, 3, 4, 5 or more times a month, etc., as discussed in more detail above).

Exemplary additional active agents are provided below.

### 1. Immune Checkpoint Inhibitors

The combination therapies and treatment regimens can be used to induce, increase, or enhance an immune response (e.g. an increase or induction of T cell response such as T cell proliferation or activation) in a subject in need thereof. Exemplary subjects include those with cancer or an infectious disease as described in more detail above. The immune response. (e.g., increased or induced T cell response) can be against a cancer or disease antigen. The immune response can be effective to treat the cancer or infection. In some embodiments, the immune response is against cancerous or disease infected cells and can reduce one or more symptoms of the cancer or disease (e.g., tumor burden, tumor progression, disease progression, etc.).

For example, the disclosed NaCl compositions can be administered in combination with one or more additional immune response stimulating or enhancing agents, for example, an checkpoint (PD1, CTLA4, TIM3, etc.) inhibitor. Thus, the one or more immune response stimulating or enhancing agents can be an additional agent that decreases an immune suppressive response in the subject. See, e.g., FIGS. 11A-11B.

### a. PD-1 Antagonists

In some embodiments, the additional active agent(s) is a PD-1 antagonist. Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example. Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In preferred embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor. Other PD-1 antagonists contemplated by the methods of this invention include antibodies that bind to PD-1 or ligands of PD-1, and other antibodies.

Suitable anti-PD-1 antibodies include, but are not limited to, those described in the following publications:
PCT/IL03/00425 (Hardy et al., WO/2003/099196)
PCT/JP2006/309606 (Korman et al., WO/2006/121168)
PCT/US2008/008925 (Li et al., WO/2009/014708)
PCT/JP03/08420 (Honjo et al., WO/2004/004771)
PCT/JP04/00549 (Honjo et al., WO/2004/072286)
PCT/IB2003/006304 (Collins et al., WO/2004/056875)
PCT/US2007/088851 (Ahmed et al., WO/2008/083174)
PCT/US2006/026046 (Korman et al., WO/2007/005874)
PCT/US2008/084923 (Terrett et al., WO/2009/073533)

Berger et al., Clin. Cancer Res., 14:30443051 (2008).

A specific example of an anti-PD-1 antibody is MDX-1106 (see Kosak, US 20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications:
PCT/US06/022423 (WO/2006/133396, pub. 14 Dec. 2006)
PCT/US07/088851 (WO/2008/083174, pub. 10 Jul. 2008)
US 2006/0110383 (pub. 25 May 2006)

A specific example of an anti-B7-H1 antibody is MDX-1105 (WO/2007/005874, published 11 Jan. 2007)), a human anti-B7-H1 antibody.

For anti-B7-DC antibodies see U.S. Pat. Nos. 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-HL. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein comprises the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119(8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

Exemplary PD-1 inhibitors include, but are not limited to,
Pembrolizumab (formerly MK-3475 or lambrolizumab, Keytruda) was developed by Merck and first approved by the Food and Drug Administration in 2014 for the treatment of melanoma.
Nivolumab (Opdivo) was developed by Bristol-Myers Squibb and first approved by the FDA in 2014 for the treatment of melanoma.
pidilizumab, by Cure Tech
AMP-224, by GlaxoSmith Kline
AMP-514, by GlaxoSmith Kline
PDR001, by Novartis
cemiplimab, by Regeneron and Sanofi

Exemplary PD-L1 inhibitors include, but are not limited to,
Atezolizumab (Tecentriq) is a fully humanised IgG1 (immunoglobulin 1 antibody developed by Roche Genentech. In 2016, the FDA approved atezolizumab for urothelial carcinoma and non-small cell lung cancer.
Avelumab (Bavencio) is a fully human IgG1 antibody developed by Merck Serono and Pfizer. Avelumab is FDA approved for the treatment of metastatic merkel-cell carcinoma. It failed phase III clinical trials for gastric cancer.
Durvalumab (Imfinzi) is a fully human IgG1 antibody developed by AstraZeneca. Durvalumab is FDA approved for the treatment of urothelial carcinoma and unresectable non-small cell lung cancer after chemoradiation.
BMS-936559, by Bristol-Myers Squibb
CK-301, by Checkpoint Therapeutics

See, e.g., Iwai, et al., Journal of Biomedical Science, (2017) 24:26, DOI 10.1186/s12929-017-0329-9.

### b. CTLA4 Antagonists

Other molecules useful in mediating the effects of T cells in an immune response are also contemplated as active agents. For example, in some embodiments, the molecule is an agent binds to an immune response mediating molecule that is not PD-1. In some embodiments, the agents target or otherwise reduce signaling through CTLA4. The agent may activities or functions similar to those described above for PD-1, but targeting CTLA4 instead of PD-1. For example, active agent may inhibits, reduces, abolishes or otherwise reduces inhibitory signal transduction through the CTLA4 receptor signaling pathway. Such decrease may result where: (i) the CTLA4 antagonist binds to a CTLA4 receptor without triggering signal transduction, to reduce or block inhibitory signal transduction; (ii) the CTLA4 antagonist binds to a ligand (e.g. an agonist) of the CTLA4 receptor, preventing its binding thereto; (iii) the CTLA4 antagonist binds to, or otherwise inhibits the activity of, a molecule that is part of a regulatory chain that, when not inhibited, has the result of stimulating or otherwise facilitating CTLA4 inhibitory signal transduction; or (iv) the CTLA4 antagonist inhibits expression of a CTLA4 receptor or expression ligand thereof, especially by reducing or abolishing expression of one or more genes encoding CTLA4 or one or more of its natural ligands. Thus, a CTLA4 antagonist can be a molecule that affects a decrease in CTLA4 inhibitory signal transduction, thereby increasing T cell response to one or more antigens.

In a preferred embodiment, the molecule is an antagonist of CTLA4, for example an antagonistic anti-CTLA4 antibody. An example of an anti-CTLA4 antibody contemplated for use in the methods of the invention includes an antibody as described in PCT/US2006/043690 (Fischkoff et al., WO/2007/056539).

Dosages for anti-PD-1, anti-B7-H1, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred.

Specific examples of an anti-CTLA4 antibody useful in the methods of the invention are Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al., Clinical Kidney Journal, 3(2):135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### c. Other Immune Checkpoint Modulators

Other immune checkpoint targets include, but are not limited to, ICOS. OX40, GITR, 4-1BB, CD40, CD27-CD70, LAG3, TIM-3, TIGIT, VISTA, B7-H3, KIR, and others, and are being targeting for cancer treatment alone and in combination with anti-PD-1, anti-PD-L1, and anti-CTLA compounds. See, for example. Iwai, et al., Journal of Biomedical Science. 24 (1): 26. doi:10.1186/s12929-017-0329-9; Donini, et al., J Thorac Dis. 2018 May; 10(Suppl 13):S1581-S1601. doi: 10.21037/jtd.2018.02.79. Thus, in some embodiments, particles are administered in combination with a compound that targets ICOS, OX40. GITR, 4-1BB, CD40, CD27-CD70, LAG3, TIM-3, TIGIT, VISTA, B7-H3, KIR, or PARP, or a combination thereof, alone or in combination with a compound that target PD-1, PD-L1, and/or CTLA.

### 2. Conventional Cancer Therapies

Additional therapeutic agents include conventional cancer therapeutics such as chemotherapeutic agents, cytokines, chemokines, and radiation therapy. The majority of chemotherapeutic drugs can be divided in to: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the new tyrosine kinase inhibitors, e.g., imatinib mesylate (GLEEVEC® or GLIVEC®), which directly targets a molecular abnormality in certain types of cancer (chronic myelogenous leukemia, gastrointestinal stromal tumors).

Representative chemotherapeutic agents include, but are not limited to, amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarbamide, idarubicin, ifosfamide, irinotecan, leucovorin, liposomal doxorubicin, liposomal daunorubicin, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, taxol and derivatives thereof, trastuzumab (HERCEPTIN®), cetuximab, and rituximab (RITUXAN® or MABTHERA®), bevacizumab (AVASTIN@), and combinations thereof. Representative pro-apoptotic agents include, but are not limited to, fludarabinetaurosporine, cycloheximide, actinomycin D, lactosylceramide, 15d-PGJ(2), and combinations thereof.

### V. Kits

Dosage units including the disclosed composition, for example, lyophilized or in a pharmaceutically acceptable carrier for shipping and storage and/or administration are also disclosed. Components of the kit may be packaged individually and can be sterile. In some embodiments, a pharmaceutically acceptable carrier containing an effective amount of the composition is shipped and stored in a sterile vial. The sterile vial may contain enough composition for one or more doses. The composition may be shipped and stored in a volume suitable for administration, or may be provided in a concentration that is diluted prior to administration. In another embodiment, a pharmaceutically acceptable carrier containing drug can be shipped and stored in a syringe.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. Any of the kits can include instructions for use.

The disclosed compositions and methods can be further understood through the following numbered paragraphs.

1. A nanoparticle formed from an alkai metal or alkaline earth metal and halide.
2. The nanoparticle of paragraph 1 wherein the alkai metal is lithium, sodium, potassium, rubidium, or cesium, and the halide is fluoride, chloride, bromide, or iodide.
3. The nanoparticle of paragraph 1 wherein alkaline earth metal is magnesium or calcium, and the halide is fluoride, chloride, bromide, or iodide.
4. The nanoparticle of paragraph 1 comprising sodium chloride, sodium fluoride, sodium bromide, sodium iodide, potassium chloride, or calcium chloride.
5. The nanoparticle of paragraph 4 comprising sodium chloride.
6. A nanoparticle formed from sodium and chloride.
7. The nanoparticle of any one of paragraphs 1-6, wherein the molar ratio of alkai metal or alkaline earth metal and halide is about 1:1.
8. The nanoparticle of any one of paragraphs 1-7, wherein the particle is cubic.
9. The nanoparticle of any one of paragraphs 1-8 further comprising a hydrophilic coating or external layer.
10. The nanoparticle of paragraph 9, wherein the layer or coating comprises amphiphilic block co-polymers, peptides, proteins, lipids, or a combination thereof.
11. The nanoparticle of paragraph 10, wherein the layer or coating comprises lipid, such as a phospholipid.
12. The nanoparticle of paragraph 6, wherein the phospholipid is a phosphoethanolamine.
13. The nanoparticle of any one of paragraphs 9-12, wherein the layer or coating comprises a PEG such as a PEG amine.
14. The nanoparticle of any one of paragraphs 9-13, wherein the layer or coating comprises or consists of a lipid-PEG conjugate such as 1,2-distearoyl-sn-glycero-3-phosphoethanolamine (DSPE) PEG (2000) Amine.
15. A pharmaceutical composition comprising a plurality of the nanoparticles of any one of paragraphs 1-14.
16. The pharmaceutical composition of paragraph 15, wherein the average hydrodynamic size of the nanoparticles is between about 10 nm and about 500 nm, or between about 25 nm and about 300 nm, or between about 50 nm and 150 nm, between about 75 nm and about 125 nm, ±5%, 10%, 15%, 20%, or 25%.
17. The pharmaceutical composition of paragraphs 15 or 16 wherein the nanoparticles are monodisperse.
18. The pharmaceutical composition of any one of paragraphs 15-17, wherein the nanoparticles are formed by a microemulsion reaction.
19. The pharmaceutical composition of paragraph 18, wherein the microemulsion reaction comprises adding molybdenum (V) chloride to a solvent solution comprising a solvent, a surfactant, and sodium oleate, and optionally free from water.
20. The pharmaceutical composition of paragraph 19, wherein the solvent is a mixture of hexane and ethanol.
21. The pharmaceutical composition of paragraphs 19 or 20, wherein the surfactant is oleylamine or oleic acid.
22. The pharmaceutical composition of any one of paragraphs 15-21 wherein the nanoparticles comprise a hydrophilic coating or external layer optionally formed by mixing the nanoparticles and a lipid-PEG conjugate together in a solvent and removing the solvent.
23. The pharmaceutical composition of any one of paragraphs 15-22 comprising a pharmaceutically acceptable carrier.
24. The pharmaceutical composition of any one of paragraphs 15-23, comprising a therapeutically effective amount of the nanoparticles.
25. The pharmaceutical composition of any one of paragraphs 15-24, comprising an effective amount to nanoparticles to reduce mitochondrial oxygen consumption rate (OCR), reduce mitochondrial respiration rate (MSR), decrease intracellular ATP level, increase the ROS level, increase levels of JNK, ERK, and/or p38 phosphorylation, increase lipid peroxidation, increase DNA damage, release of cytochrome c, increase of caspase-3 activity, increase caspase-1 activity, increase cell swelling and/or bleb formation, induce cell rupture and/or complete osmotic lysis, increase NLRP3 inflammasome induction, increase GSDMD N-terminal fragment release, elevate IL-1β secretion, increase intracellular K⁺ level, or a combination thereof in tumor cells and/or cancer cells.
26. The pharmaceutical composition of any one of paragraphs 15-25, comprising an effective amount to nanoparticles to increase apoptosis, necrosis, and/or pyroptosis of tumor and/or cancer cells.
27. The pharmaceutical composition of paragraphs 25 or 26 wherein mitochondrial oxygen consumption rate (OCR), reduce mitochondrial respiration rate (MSR), decrease intracellular ATP level, increase the ROS level, increase levels of JNK, ERK, and/or p38 phosphorylation, increase lipid peroxidation, increase DNA damage, release of cytochrome c, increase of caspase-3 activity, increase caspase-1 activity, increase cell swelling and/or bleb formation, induce cell rupture and/or complete osmotic lysis, increase NLRP3 inflammasome induction, increase GSDMD N-terminal fragment release, elevate IL-1γ secretion, increase intracellular K⁺ level, increase in apoptosis, increase in necrosis, increase in pyroptosis, or any combination thereof is altered or effected to a greater degree in tumor cells and/or cancer cells relative to non-tumor and/or non-cancer cells.
28. The pharmaceutical composition of any one of paragraphs 15-27, in dosage form suitable for administrating about 0.1 mg/kg to about 1,000 mg/kg, or about 1 mg/kg to about 100 mg/kg, or about 5 mg/kg to about 50 mg/kg to a subject in need thereof.
29. The pharmaceutical composition of any one of paragraphs 15-28 comprising one or more additional active agents.
30. The pharmaceutical composition paragraph 29, wherein the one or more additional active agents comprises an immune checkpoint inhibitor, a chemotherapeutic agent, or a combination thereof.
31. The pharmaceutical composition of paragraph 30 comprising an immune checkpoint inhibitor selected from PD-1 antagonists, CTLA4 antagonists, and a combination thereof.
32. The pharmaceutical composition of paragraph 31, wherein the PD-1 antagonist and/or CTLA antagonist is an antibody or antigen binding fragment thereof.
33. A method of making antigen comprising contacting cancer cells with an effective amount of the nanoparticle of any one of paragraphs 1-14, or the pharmaceutical composition of any one of paragraphs 15-32 to induce death of the cells.
34. The method of paragraph 33, wherein the nanoparticles are effective to increase expression or secretion of one or more damage-associated molecular pattern (DAMP) molecules.
35. The method of paragraph 34, wherein the DAMP molecule(s) comprise calreticulin (CRT), adenosine triphosphate (ATP), high mobility group box 1 (HMGB1), and combinations thereof.
36. The method of any one of paragraphs 33-35 wherein the contacting occurs in vitro or ex vivo.
37. The method of any one of paragraphs 33-36, wherein the cancer cells are isolated from a subject in need of cancer treatment or prevention.
38. An antigen comprising dying or dead cells, or a lysate, extract, fraction, isolate, or collection of secreted factors thereof formed according to the method of any one of paragraphs 33-37.
39. A method of vaccinating a subject comprising administering a subject in need thereof an effective amount of the antigen of paragraph 38 to increase or induce an immune response to the antigen.
40. The method of paragraph 39 comprising administering the subject the pharmaceutical composition of any one of paragraphs 15-32.
41. The method of paragraphs 39 or 40 further comprising administering the subject an adjuvant.
42. The method of paragraphs 40 or 41, wherein any combination of the antigen, the pharmaceutical composition, and adjuvant are administered together.
43. The method of paragraph 42, wherein any combination of the antigen, the pharmaceutical composition, and adjuvant are part of the same or different admixtures.
44. The method of paragraphs 40 and 41, wherein any combination of the antigen, the pharmaceutical composition, and adjuvant are administered separately.
45. The method of any one of paragraphs 39-44, wherein the subject has cancer.
46. A method of treating cancer comprising administering to a subject in need thereof the pharmaceutical composition of any one of paragraphs 15-32.
47. The method of paragraph 46, wherein the pharmaceutical composition induces an immune response to the cancer in the subject.
48. The method of any one of paragraphs 45-47, wherein the subject has a bone, bladder, brain, breast, cervical, colo-rectal, esophageal, kidney, liver, lung, nasopharyngeal, pancreatic, prostate, skin, stomach, or uterine cancer.
49. The method of any one of paragraphs 39-48 wherein the administration is be injection or infusion.
50. The method of any one of paragraphs 39-49, wherein the administration is local to the site in need of treatment.
51. The method of paragraph 50, wherein the site is a tumor.
52. The method of any one of paragraphs 39-51, wherein the administration is systemic.
53. The method of any one of paragraphs 39-52, further comprising administration of one or more additional active agents.
54. The method of paragraph 53, wherein the one or more additional active agents comprises an immune checkpoint inhibitor, a chemotherapeutic agent, or a combination thereof.
55. The method of paragraph 54 comprising an immune checkpoint inhibitor selected from PD-1 antagonists, CTLA4 antagonists, and a combination thereof.
56. The method of paragraph 55, wherein the PD-1 antagonist and/or CTLA antagonist is an antibody or antigen binding fragment thereof.
57. The method of any one of paragraphs 53-56, wherein the particles and the additional active agent are administered to the subject at different times.
58. The method of any one of paragraphs 53-56, wherein the particles and the additional active agent are administered to the subject at the same time.
59. The method of any one of paragraphs 53-56, wherein the particles and the additional active agent form part of the same pharmaceutical composition.
60. The method of any one of paragraphs 46-59, wherein the particles are administered to the subject by intravesical instillation, optionally wherein the subject has bladder cancer.

The present invention can be further understood by reference to the following non-limiting examples.

### EXAMPLES

Jiang, et al., “NaCl Nanoparticles as a Cancer Therapeutic,” Adv Mater. 2019 November; 31(46):e1904058. doi: 10.1002/adma.201904058. Epub 2019 Sep. 25, and the Supporting Information associated therewith is specifically incorporated by reference herein in their entireties.

The Examples below as well as the other disclosure herein utilize the following abbreviations:

Abbreviation list

| | |
|---|---|
| NPs | Nanoparticles |
| SCNPs | Sodium Chloride Nanoparticles |
| PSCNPs | Phospholipid-coated Sodium Chloride Nanoparticles |
| RB- | Rhodamine B-labeled PSCNPs |
| PSCNPs | |
| TEM | Transmission Electron Microscopy |
| SEM | Scanning Electron Microscopy |
| EDS | Energy Dispersive Spectroscopy |
| XRD | X-ray Diffraction |
| FT-IR | Fourier-transform Infrared |
| DMEM | Dulbecco's Modified Eagle's Medium |
| RPMI | Roswell Park Memorial Institute Medium 1640 |
| 1640 | |
| SBFI-AM | Sodiumbinding Benzofuran Isophthalate |
| | Acetoxymethyl Ester |
| MQAE | N-(Ethoxycarbonylmethyl)-6-Methoxy-Quinolinium |
| | Bromide |
| PBFI-AM | Potassium-Binding Benzofuran Isophthalate |
| | Acetoxymethyl Ester |
| OCR | Oxygen Consumption Rate |
| MSR | Mitochondrial Respiration Rate |
| ROS | Reactive Oxygen Species |
| PBS | Phosphate-buffered Saline |
| EthD-III | Ethidium homodimer III |
| [Na⁺]ᵢₙₜ | Intracellular sodium concentration |
| [K⁺]ᵢₙₜ | Intracellular potassium concentration |
| PBMCs | Peripheral blood mononuclear cells |
| TDLNs | Tissue draining lymph nodes |
| DMAPs | Damage associated molecular patterns |
| ATP | Adenosine Triphosphate |
| HMGB-1 | High-mobility group box 1 protein |
| APCs | Antigen-presenting cells |
| DCs | Dendritic cells |
| 1CD | Innnunogenic cell death |
| F/T | Freeze thaw |

Statistical Analysis

For in vitro study, all measurements were performed in sextuplicate unless specified otherwise. Data obtained from high-content BioApplication Studio 2.0 were exported and further analyzed using a JMP statistical analysis package (SAS Institute, North Carolina). Half-maximum inhibitory concentration (IC₅₀) was determined by Doseresp using Origin 9. The median lethal concentrations (LC₅₀) were calculated with a curve-fitting program using GraphPad Prism 5 (San Diego, Calif.). Measured values were presented as mean±SD. One tailed Student's t test was used for comparison among groups, with P values of 0.05 or less representing statistical significance.

### Example 1: NaCl Nanoparticle Synthesis and Degradation

### Materials and Methods

Synthesis of Sodium Chloride Nanoparticles (SCNPs).

In a typical synthesis, 20 mg of sodium oleate (TCI, 97%, Lot No.: W76EGFQ), 1 mL of oleylamine (70%, Sigma-Aldrich, Lot No.: STBF9554V) and 50 mg 1,2-tetradecanediol (90%, Sigma-Aldrich) were dissolved in a mixed solution containing 10 mL hexane (99.9%. Fisher) and 10 mL ethanol (99.9%, Fisher). Into the mixture, 15 mg of molybdenum (V) chloride (95%, Sigma-Aldrich, Lot No.: MKBQ9967V) was added and the solution was magnetically stirred for 24 h at 60° C. The raw products were collected by centrifugation at 12000 RPM for 10 min. The particles were redispersed in hexanes with brief sonication and the centrifugation/hexane washing process was repeated 3 times to remove unreacted precursors.

Phospholipid-Coated Sodium Chloride Nanoparticles (PSCNPs).

The above-synthesized SCNPs (10 mL) in hexane were sonicated for 30 s and mixed with 80 μL phospholipid solution (1 mg/mL) DSPE-PEG (2000) Amine (1,2-distearoyl-sn-glycero-3-phosphoethanolamine-N-[amino(polyethylene glycol)-2000] (ammonium salt), Avanti, Lot No.: 180PEPEPEG2NH2-64). For rhodamine B labeled PSCNPs (RB-PSCNPs), Liss Rhod PE (1,2-dipalmitoyl-sn-glycero-3-phosphoethanolamine-N-(lissamine rhodamine B sulfonyl, ammonium salt, Avanti, Lot No.: F160LRPE-33) solution in chloroform (40 μL, 1 mg/mL) was added into 10 mL SCNPs as well. The mixture was allowed to sonicate for 30 s. The solvent was removed under reduced pressure at 40° C. using a Buchi R II Rotavapor. 10 mL PBS/water was then added to the flask and the mixture was sonicated for 30 s. Fresh-made PSCNPs were used for characterizations, in vitro and in vivo studies unless specified otherwise. All the particle doses were calculated based on NaCl concentration unless specified otherwise.

Characterizations of NPs.

X-ray diffraction (XRD) analysis was carried out on a Bruker D8-Advance using dried samples placed on a cut glass slide with Cu Kα1 radiation (λ=1.5406 Å). Scanning electron microscopy (SEM) and energy dispersive X-ray spectra EDS elemental mapping images were acquired on a FEI Teneo field emission SEM equipped with an Oxford EDS system. Transmission electron microscopy (TEM) was carried out on an FEI Tecnai20 transmission electron microscope operating at an accelerating voltage of 200 kV. High resolution TEM analysis was performed on a Hitachi transmission electron microscope H9500 operating at a 300 kV accelerating voltage. Particle size and zeta potential measurements were carried out on a Malvern Zetasizer Nano ZS system. Fourier-transform infrared (FT-IR) spectra were recorded on a Nicolet iS10 FT-IR spectrometer.

Stability and Release Experiments.

PSCNPs were dispersed in 100 μl ammonium acetate buffers (pH=5.5 or pH=7) and added into a Slide-A-Lyzer™ MINI Dialysis Device (MWCO=2K, Cat #69550, Thermofisher, US). Place the unite into a 5 mL Eppendorf tube containing 4.5 mL ammonium acetate buffer. Keep the tube on the shaker (20 rpm) at room temperature. At different time points (0, 10 min, 0.5, 1, 2, 4, 6, 12, 24 h), take 400 μL of PSCNPs solution from the Eppendorf tube to test the free ions concentration. A Na+ electrode (HORIBA LAQUAtwin Na-11) was used to measure free Na+ ions, while MQAE (N-(ethoxycarbonylmethyl)-6-methoxy-quinolinium bromide, Setareh Biotech, Lot No.: 50610) were used to measure the free Cl− ions in the solution. All measurements were performed following the manufacture's protocol and repeated in sextuplicate.

### Results

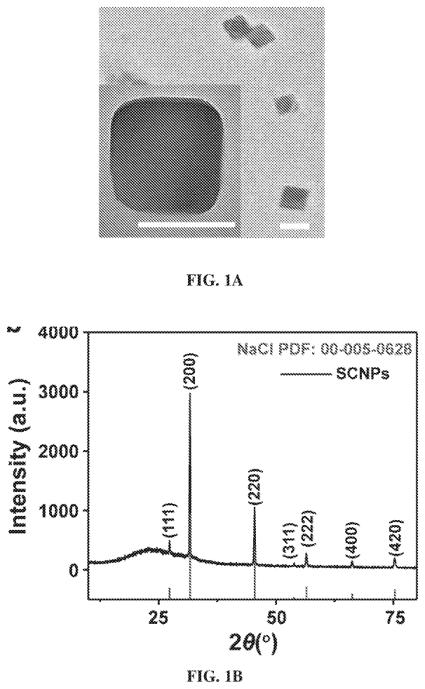

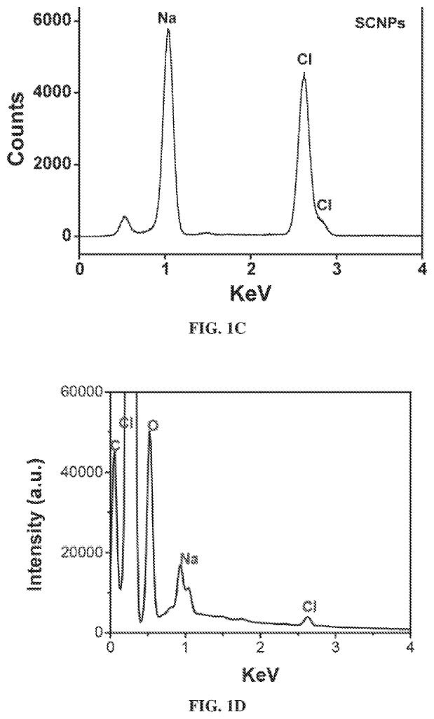

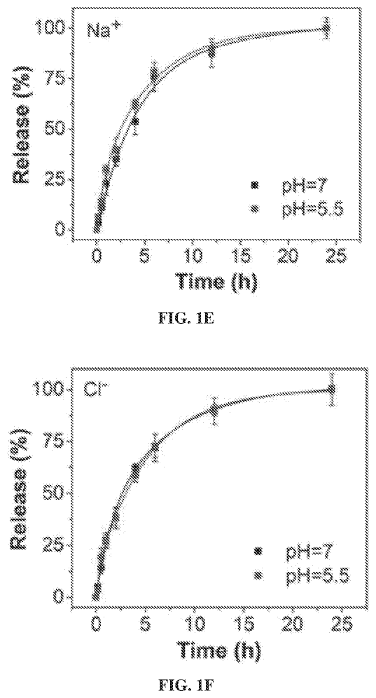

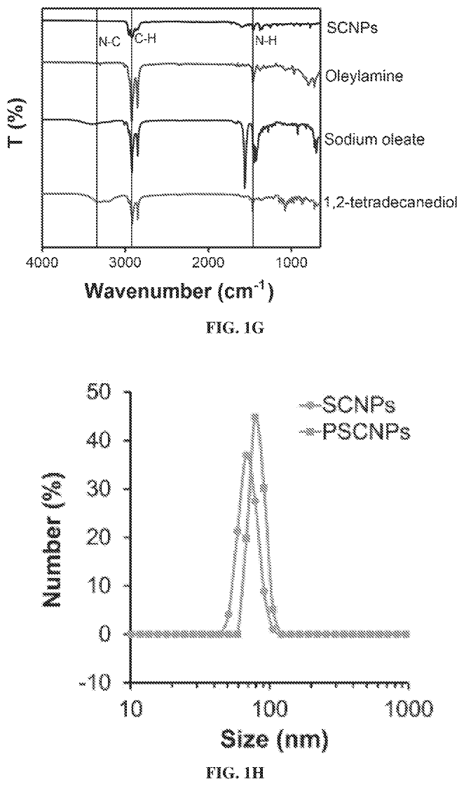

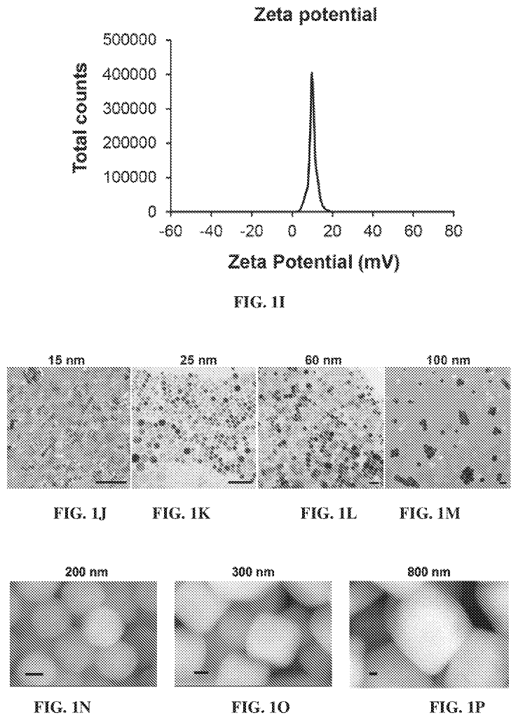

Sodium chloride nanoparticles (SCNPs) were synthesized through a microemulsion reaction. The reaction took place in a hexane/ethanol mixed solvent, with sodium oleate and molybdenum chloride as sodium and chloride precursors, and oleylamine as a surfactant. A typical reaction yields ≈77±10.6 nm SCNPs as determined by transmission electron microscopy (TEM) (FIG. 1A). Dynamic light scattering (DLS) found that their hydrodynamic size was ˜84.6±9.8 nm NaCl nanoparticles with narrow size distribution (FIG. 1H). Other sizes of NaCl nanoparticles (15 to 800 nm) can be prepared by tuning reaction conditions, and monodispersed particles of about 15 nm, about 25 nm, about 60 mn, and about 100 nm, about 200 nm, about 300 nm, and about 800 nm were made to illustrate the foregoing (FIGS. 1J-1P). X-ray powder diffraction (XRD) found that the crystal structure of the particles was cubic phase NaCl (Fm-3m, PDF No.: 00-005-0628, FIG. 1B). Energy dispersive spectroscopy (EDS) confirmed that sodium and chloride molar ratio was ˜1:1 in the product (FIGS. 1C, 1D, Table 1), with negligible impurities including molybdenum.

TABLE 1
EDS analysis spectrum (FIG. 1D) confirmed
that Na to Cl atom molar ratio was ~1:1.

| | | Atom |
|---|---|---|
| Element | Wt % | ratio |
| Na | 37.2 | 1.0 |
| Cl | 62.8 | 1.1 |

The as-synthesized NaCl nanoparticles are hydrophobic because of the oleylamine coating (Fourier transform infrared spectroscopy. FIG. 1G). To transfer nanoparticles into aqueous solutions, a layer of PEGylated phospholipid, DSPE-PEG2000 amine, was imparted onto the nanoparticle surface. The resulting, phospholipid coated NaCl nanoparticles (designated as PSCNPs), can be well dispersed in aqueous solutions, and they bore a hydrodynamic size of 98.0±13.1 nm (FIG. 1H) compared to un-coated SCNPs and a positive surface charge of +9.7 mV (FIG. 1I). The phospholipid coating renders NaCl nanocrystals with extended lifetimes in water but does not stop the process of disintegration. Indeed, TEM analysis found small cavities on the nanocrystal surface when PSCNPs were incubated in water for 1-6 h. Further incubation led to significant particle disintegration (reduction into smaller pieces after 6 h) and eventually complete dissolution by 24 hours.

To better understand the process, ion release was assessed in sodium- and chlorine-free ammonium acetate buffers (pH=7.0 or 5.5) using SBFI-AM and MQAE as Na⁺ and Cl⁻ sensors, respectively. Comparable release profiles for the two ions both reaching a plateau at ˜12 h (FIGS. 1E, 1F). Notably, lowering pH to 5.5 did not accelerate nanoparticle degradation (FIGS. 1E, 1F).

### Example 2: NaCl Nanoparticles are Taken Up by Cells and can be Cytotoxic

### Materials and Methods

Cell Culture.

4T1 (murine mammary carcinoma). HT29 (human colorectal adenocarcinoma), A549 (human lung carcinoma), SGC7901 (human gastric adenocarcinoma), PC-3 (human prostate adenocarcinoma), UPPL-1541, (murine bladder carcinoma), t24, UMUC2 cells were grown in RPMI-1640 (Corning, 10-040-CV). U87MG (human glioblastoma) and RAW264.7 cells (murine macrophage) were grown in DMEM (Corning, 10-013-CV). B16-F10 (murine melanoma) and BBN963 cells were grown in high glucose DMEM (ATCC® 30-2002™). SCC VII cells (murine head and neck squamous carcinoma) were grown in Corning® DMEM (Dulbecco's Modified Eagle's Medium)/Hams F-12 50/50 Mix (Corning, 10-090-CV). All the cell culture medium were supplemented with 10% fetal bovine serum (FBS) and 100 units/mL of penicillin and 100 units/mL streptomycin (MediaTech, USA). Human primary prostate epithelial cells (HPrECs, ATCC, PCS440010) were maintained in serum free conditions with prostate epithelial cell growth kit (ATCC PCS440040). Murine primary urothelial epithelial cells K1970 were maintained in DMEM/F12 70/30 medium. This medium also contains hydrocortisone (1000λ), insulin (5 mg/ml), fungizone (250 μg/ml), gentamicin (10 mg/ml) cholera toxin (11.7 μM) and Y-27632 mM. Primary prostate epithelial cells (HPrECs, ATCC, PCS440010) were maintained in serum free conditions with prostate epithelial cell growth kit (ATCC PCS440040). The mouse spermatogonial cell line (C18-4) was established from germ cells isolated from the testes of 6-day old Balb/c mice (Hofmann et al., Stem Cells 23, 200-210 (2005), and the cells were cultured in DMEM (Corning, 10-013-CV) containing 5% FBS, and 100 U/ml streptomycin and penicillin. All cells were maintained in a humidified, 5% carbon dioxide atmosphere at 37° C.

M7T Assay to Study Cytotoxicity.

Cells were seeded into 96-well plates at the density of 1×10₄ cells per well and incubated overnight. Then the cells were treated with PSCNPs dispersed in PBS, PSCNPs pre-aged in PBS (1, 3, 8 and 24 h), or NaCl salt at a dose range of 3.25-320 μg·NaCl/mL for 24 h. MTT assays (3-(4,5-dimethylthiazol-2-yl)-2,5-diphenyl tetrazolium bromide, Sigma) were performed following the manufacture's protocol. The absorbance at 570 nm was measured by a microplate reader (Synergy Mx, BioTeK). All measurements were performed in sextuplicate.

Live/Dead Assay to Assess Time-Dependent Cytotoxicity.

The time-dependent cytotoxicity of PSCNPs was evaluated using a live/dead viability/cytotoxicity kit (Biotum, Cat No.: 30002). After incubating with PSCNPs at a dose of 52.5, 105.0 or 160.0 μg/mL (NaCl concentration, the same below), PC-3 cells were washed with PBS twice and stained with 2 μM Calcein AM and 3 μM of PI for live and dead cells detection, respectively. All the cells were co-stained with 10 μM Hoechst 33342 (Life Technologies) for nucleus observation. Quantitative time-lapse fluorescence microscopy was conducted and sequential images were automatically acquired on an Arrayscan™ VTI HCS reader using the HCS Studio™ 2.0 Target Activation BioApplication module (Thermo Scientific, MA) at 0, 2, 4, 6, and 12 h after treatment with PSCNPs. PBS and 10 μM CdCl₂ (termed as Cd) were analyzed as a negative and a positive control, respectively. For all measurements, 49 fields per well and approximately 5000 cells were analyzed using a 40× objective (NA 0.5), a Hamamatsu ORCA-ER digital camera in combination with a 0.63× coupler, and Carl Zeiss microscope optics in an auto focus and high resolution mode. Channel two (Ch2) used a BGRFR 485-20 filter for calcein AM dye (live cell) imaging. Channel three (Ch3) used a BGRFR 549-15 filter for ethidium homodimer-III (dead cell) imaging. High-content multichannel analysis (HCA) was analyzed using HCS Studio 2.0 Target Activation BioApplication (Thermo Scientific, MA).

Intracellular Concentrations of PSCNPs, Na⁺, Cl⁻, K⁺ Fluorescence Staining, Image Acquisition and High-Content Analysis.

Microscope studies were carried out on a Cellomics® ArrayScan® VTI HCS Reader with a live cell chamber and the HCS Studio™ 2.0 Cell Analysis Software (Thermo Scientific). For all measurements, 49 fields per well and approximately 5000 cells were analyzed using a 40× objective (NA 0.5), a Hamamatsu ORCA-ER digital camera in combination with a 0.63× coupler, and Carl Zeiss microscope optics in auto focus and high resolution mode with three channels. Image smoothing was applied to reduce object fragmentation prior to object detection. Channel one (Ch1) used the BGRFR 386-23 filter for Hoechst 33342 staining that was used for auto-focus, object identification, and segmentation. Ch2 used a BGRFR 485-20 filter for SBFI-AM, PBFI-AM (potassium-binding benzofuran isophthalate acetoxymethyl ester, Setareh Biotech, Lot No.: 5027), and MQAE imaging. Ch3 used a BGRFR 549-15 filter for RB-PSCNPs imaging. High-content multichannel analysis (HCA) was analyzed using HCS Studio 2.0 Target Activation BioApplication (Thermo Scientific, MA). Single-cell based HCA provided multiple parameters to characterize the nucleus, the number of cells, and total or average intensity of each cell. Total intensity was defined as all pixels within a cell. Average intensity was defined as all the pixels within a cell divided by the total area of the cell. Specifically, for PSCNP cellular uptake, PC-3 cells were incubated with RB-PSCNPs for 0, 2, 4 and 6 h. Then, LysoTracker® Green DND-26 (molecular probes) and Hoechst 33342 dyes were co-stained for 10 min. Fluorescent images were obtained every 10 min. All measurements were performed in sextuplicate. For intracellular Na⁺, Cl⁻ and K⁺ characterization, PC-3 cells were treated with RB-PSCNPs for 0, 2.4 and 6 h. The cells were then incubated with 10 μM SBFI-AM in 0.04% Pluronic F-127 (Sigma, Lot No.: SLBB4267V), 10 mM MQAE, or 10 μM PBFI-AM in 0.04% Pluronic F-127, respectively for Na⁺, Cl⁻ and K⁺ staining. The final fluorescence signal was measured by Ch2.

### Results

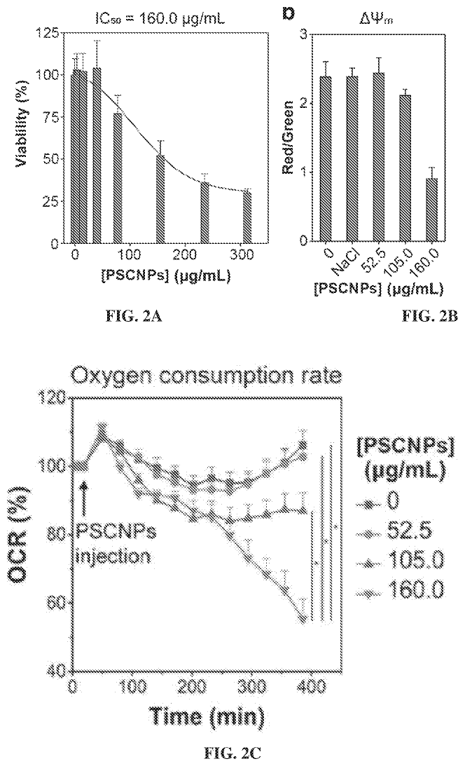

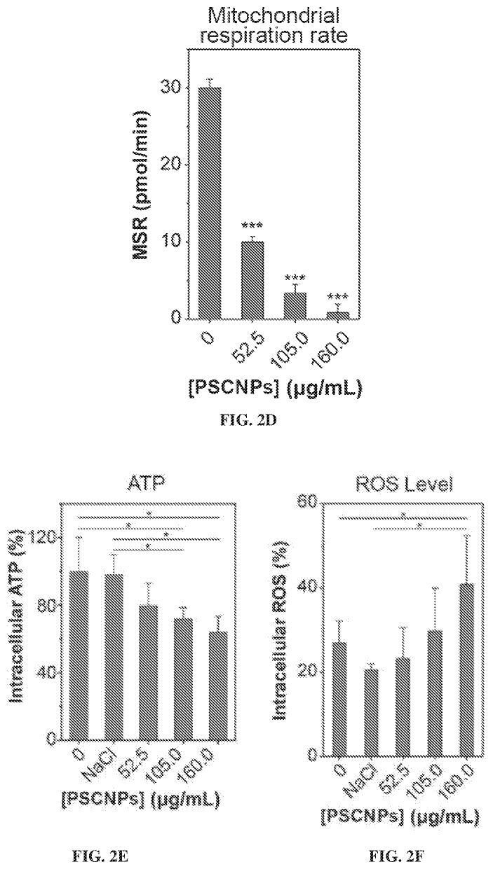

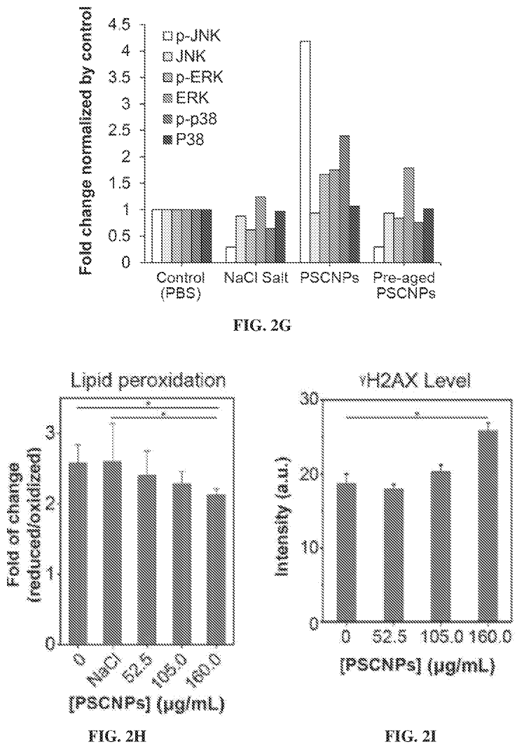

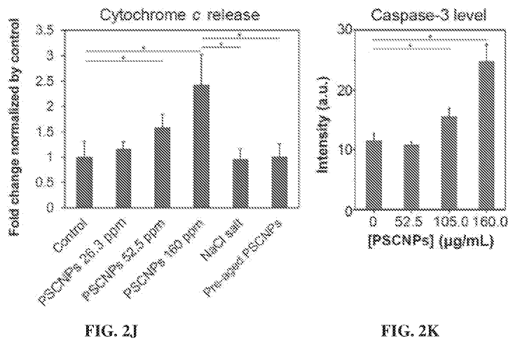

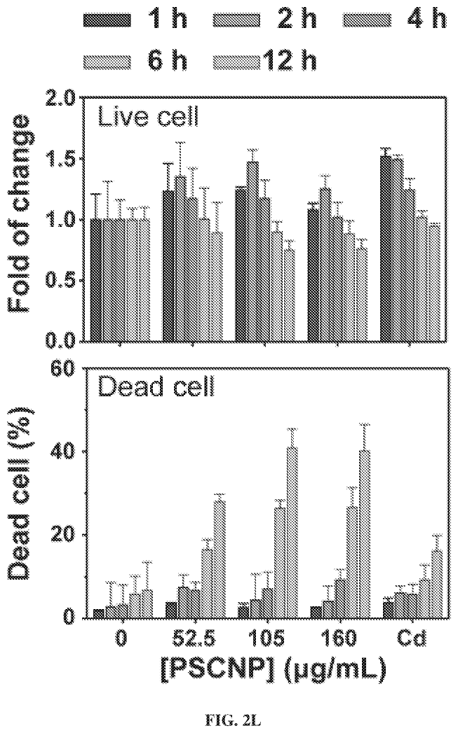

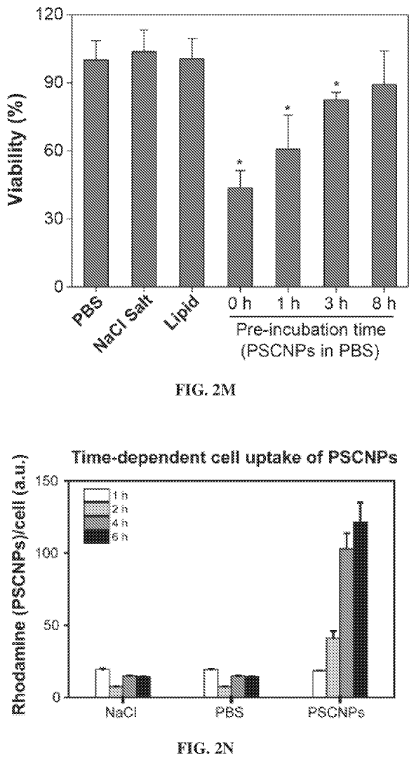

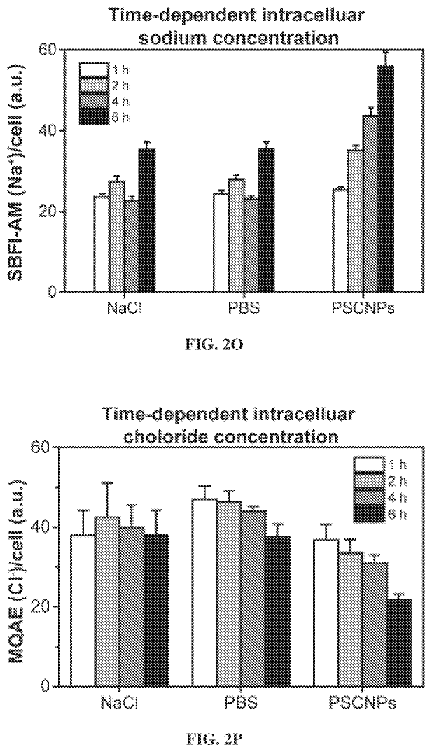

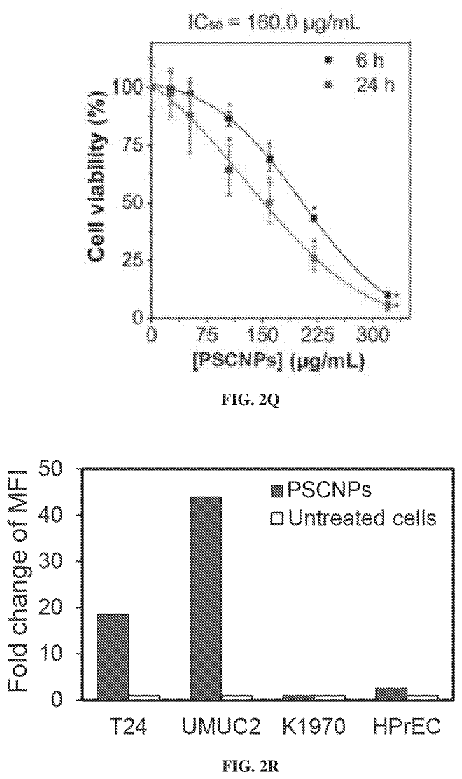

The impact of PSCNPs on cell viability was studied, starting with PC-3 cells, a human prostate adenocarcinoma cell line. MTT assay found a remarkable cytotoxicity with PSCNPs, showing an IC₅₀ of 160.0 μg/mL (NaCl concentration, the same below; FIG. 2A, FIG. 2Q). Similar results were observed with Calcein AM/PI live/dead assays (FIG. 2L). As a comparison, NaCl salt at 160 μg/mL and free phospholipid showed no toxicity to PC-3 cells (FIG. 2M). The cytotoxicity was mitigated when PSCNPs were aged before cell incubation. For instance, when PSCNPs were incubated in PBS for 1, 3, and 8 h before being added to a culture medium, the cell viability increased to 60.6%, 82.4%, and 89.2%, respectively; when the pre-incubation time exceeded 8 h, the particles became completely non-toxic to cells (FIG. 2M). These observations indicate that the cytotoxicity of PSCNPs is associated with the NaCl nanocrystal but not the constituent electrolytes or phospholipids.

The uptake of PSCNPs by cells and their fate inside them was examined. PSCNPs were labeled with rhodamine B and cell endosomes/lysosomes was stained with LysoTracker. Time-relapse live cell images were collected and analyzed the fluorescence intensities of each individual cell (n=5000). Time- and concentration-dependent increase of intracellular rhodamine B signals, and good spatial overlap between the rhodamine B and LysoTracker signals were observed (FIG. 2N). This indicates that PSCNPs were taken up by cells through endocytosis, which is consistent with observations by others with different phospholipid-coated nanoparticles (Oh and Park, Int J Nanomed 9, 51-63 (2014)). Meanwhile, SBFI-AM and MQAE staining found a consistent increase of intracellular Na+ (FIG. 2O) and Cl− concentrations ((2P) notably, the MQAE signals are reversely correlated with Cl− concentrations (Kim, et al., BMC Neurosci. 16, 90 (2015)). Generalized linear regression analysis also showed good correlation between rhodamine B and SBFI-AM or MQA signals, indicating that PSCNPs were gradually degraded inside cells and released the constituent ions.

FIG. 2R is a bar graph illustrating the cellular uptake of NaCl NPs in cancer cell lines, T24 and UMUC2, and normal cell lines. K1970 and HPrEC.

### Example 3: NaCl Nanoparticles Induce Cancer Cell Apoptosis

### Materials and Methods

Mitochondrial Electric Potential (ΔΨₘ).

The change of mitochondrial membrane potential was measured by a JC-1 mitochondrial membrane potential detection kit (Biotium, Cat No.: 30001). The JC-1 working solution was prepared by adding 10 μL of the concentrated dye to 1 mL of FBS-free RPMI medium. PSCNPs (52.5, 105, or 160 μg/mL), PBS, and NaCl (160.0 μg/mL in PBS) were incubated with cells for 6 h. The medium was removed and replaced with the JC-1 working solution and the incubation took another 15 min. The stained cells were analyzed on an Array Scan VII reader by analyzing Ch2 (green, JC-1 monomeric dye), and Ch3 (red, JC-1 aggregated dye) signals. The red/green ratio was analyzed by HCS Studio 2.0 Target Activation BioApplication software (Thermo Scientific, MA).

Oxygen Consumption Rates (OCR).

PC-3 cells (20,000/well) were seeded in Seahorse XFe 24 assay plates and cultured in 250 μL of RPMI1640 medium overnight. Cells were washed and incubated with Seahorse base medium supplemented with 2 mg/mL of glucose, 1 mM of glutamine, and 1 mM of sodium pyruvate (pH 7.4) for 1 h. After 3 consecutive measurements of basal metabolic rates, PSCNPs (52.5, 105, or 160 μg/mL) or PBS was mixed with the cells. The metabolic rates were measured every 30 min up to 6 h. For each measurement, the cells were sequentially treated with 2 μM of oligomycin, 3 μM of FCCP (Carbonyl cyanide 4-(trifluoromethoxy) phenylhydrazone), and 3 μM antimycin/3 μM rotenone and analyzed 3 times for each stage. Respiration rate in support of ATP production was calculated as OCR differences before/after the oligomycin treatment. All measurements were performed in sextuplicate.

ATP Level.

Luminescent ATP detection assay kit (Abcam, ab113849) was used to determine cellular ATP contents following the manufacturer's protocol. PC-3 cells were grown in a 96-well plate at the density of 1×10⁴ cells per well, and were incubated with various concentrations of PSCNPs (52.5, 105, or 160 μg/mL NaCl μg/mL) for 6 h. 50 μL of Lysis buffer was added into each well and incubated for 5 min under shaking on an orbital shaker at 700 RPM. Then, 50 μL of the reconstituted substrate solution was added into each well and the mixture was shaken for 15 min in dark. The luminescence intensity of each well was measured on a microplate reader (Synergy Mx, BioTeK) and normalized to that in control cells.

ROS Generation and Lipid Peroxidation.

PC-3 cells were subcultured in a 96-well plate at the density of 1×10⁴ cells per well, then were incubated with PSCNPs at a concentration of 52.5, 105.0, or 160 μg/mL for 4 h. The treated cells were incubated with 10 μM of DCFH-DA (2′,7′-dichlorofluorescin diacetate, Sigma) and the 529-nm fluorescence intensity was measured on a microplate reader (Synergy Mx, BioTeK). Cells were incubated with PSCNPs at a concentration of 52.5, 105, or 160 μg/mL for 6 h for lipid peroxidation analysis. The treated cells were incubated with 10 μM of lipid peroxidation sensor (Life technologies) for 30 min in complete growth medium at 37° C. The cells were washed once with PBS and then the fluorescence intensity of the reduced state (red, ex/em: 530/590 nm) and oxidized state (green, ex/em: 488/560 nm) were analyzed. The data were represented as red/green fluorescence intensity ratios.

DNA Damage and Caspase-3 Activation.

γ-H2AX and caspase-3 double immunostainings were preformed to confirm DNA damage and the activation of caspase-3 apoptotic pathway. PC-3 cells were seeded in a 96-well plate at a density of 1×10⁴ cells per well and cultured overnight. Cells were then incubated with PSCNPs at a dose of 52.5, 105 or 160 μg/mL for 24 h. The treated PC-3 cells were fixed with 4% paraformaldehyde for 30 min at room temperature, followed by 3 repeated washes with PBS. After fixation, the cells were permeabilized by 0.1% Triton X-100 in PBS, incubated with a mouse anti-phospho-Histone-H2AX antibody (Ser139, γ-H2AX, Millipore, Mass.), and a mouse anti-cleaved-caspase-3 antibody (Cell Signaling, #9664) in a PBS/BSA/0.5% Tween 20 solution at 4° C. overnight. After washing twice with PBS/BSA, the cells were incubated with goat anti-rabbit Dylight 650, mouse anti-rabbit Dylight 488 (Thermo Scientific, MA), and Hoechst 33342 in a PBS/BSA solution for 90 min at room temperature. Flow cytometry (Beckman Coulter CytoFLEX) was conducted for signal quantification.

Cytochrome e Release Induced by PSCNPs.

PC-3 cells were seeded in 2-well chamber slide (Nunc™ Lab-Tek™ II Chamber Slide™ System, ThermoFisher) at the density of 4×10⁵ cells per well for overnight growth. Then the cells were incubated with PSCNPs at a concentration of 26.3, 52.5 or 160 μg/mL for 6 h. Cytochrome c were analyzed by ApoTrack™ Cytochrome c Apoptosis ICC Antibody Kit (ab110417) following manufacture's protocol. Confocal images were taken at 100× magnification on a Zeiss LSM 710 Confocal Microscope and analyzed by ImageJ to compare the fluorescence intensity.

Western Blot Analysis.

Antibodies used were phospho-JNK1/2 (Thr183/Tyrl85) (Cell Signaling; 4668), JNK1/2 (Cell Signaling; 9252), phospho-ERK1/2 (Cell Signaling; 4370), ERK1/2 (Cell Signaling; 4695), phospho-p38 MAPK (Cell Signaling; 4511), p38 MAPK (Cell Signaling; 8690), cleaved caspase-3 (Cell Signaling, 9661), α-Tubulin (Abcam, 7291), NLRP3 (D4D8T) Rabbit mAb (Cell Signaling, 15101). PC-3 cells were incubated with PSCNPs at a concentration of 160 μg/mL for 6 h. The cells were then analyzed for cell stress, in particular the impact on the JNK/p38 MAPK pathways. PBS, NaCl solution (160 μg/mL), and PSCNPs pre-aged in PBS (160 μg/mL) were used as negative controls. For NLRP3 inflamasome studies, PC-3 cells were incubated with PSCNPs at a concentration of 40 or 80 μg/mL for 2 h. PBS, NaCl solution (80 μg/mL), and PSCNPs pre-aged in PBS (80 μg/mL) were used as negative controls. Cell lysates were prepared by homogenizing cells in a RIPA buffer supplemented with 1× proteinase inhibitor cocktail (Amresco). Protein concentration was determined using a bicinchoninic acid (BCA) protein assay kit (Thermo Fisher Scientific). Protein lysates were loaded onto 10% SDS-PAGE and were transferred to PVDF membrane. Nonspecific binding to the membrane was blocked by incubation with 5% nonfat milk at room temperature for 1 h. The membranes were incubated for 16 hours at 4° C. with primary antibodies at the dilutions specified by the manufacturers. After secondary antibody incubation for 1 h at room temperature, membranes were treated with ECL reagents (Thermo Fisher Scientific) and exposed to X-ray films (Santa Cruz). All the imaging results were analyzed by ImageJ.

### Results

The increase of intracellular osmolarity would extensively affect cell functions. One of the most susceptible organelles is mitochondrion, whose membrane potential (ΔΨₘ) is sensitive to cytosol osmolarity changes. Indeed, JC-1 staining found that ΔΨₘ was largely depolarized when cells were incubated with 160.0 μg/mL PSCNPs for 2 h (FIG. 2B). This led to a halt of the mitochondrial functions. Specifically, Seahorse mitochondrial stress assay showed that mitochondrial oxygen consumption rate (OCR) and mitochondrial respiration rate (MSR) were reduced by 47.9% and 91.0%, respectively, within 6 hours of incubation with 160.0 μg/mL PSCNPs (FIG. 2C, 2D). The reduced OCR and MSR in turn affects ATP and reactive oxygen species (ROS) production at the Complex I and III of the mitochondrial respiratory chain. Relative to control cells, the intracellular ATP level was decreased by 36.0% at 4 h (FIG. 2E), and the ROS level was increased by 22.3% (FIG. 2F). Western blotting found significantly increased levels of JNK, ERK, and p38 phosphorylation (FIG. 2G), which are signs of elevated oxidative stress (Benhar et al., EMBO Rep. 3, 420-425 (2002), Mates et al., Arch. Toxicol. 86, 1649-1665 (2012)). This was further corroborated by the detection of extensive lipid peroxidation (FIG. 2H) and DNA damage (γH2AX staining, FIG. 2I) in PSCNP treated cells. On the other front, the dissipated mitochondrion membrane led to the release of cytochrome c (FIG. 2J). All these impacts converged on the induction of cell apoptosis, indicated by a significant increase of caspase-3 activity at 24 h (FIGS. 2G, 2K).

### Example 4: NaCl Nanoparticles Induce Cancer Cell Pyroptosis

### Materials and Methods

Cell Morphology Changes and Cell Expansion.

The PC-3 cell morphology changes were monitored by taking bright-field images every 20 minutes between 2 and 6 h of incubation with PSCNPs (160.0 μg/mL) on a Cellomics® ArrayScan® VTI HCS Reader. A time-lapse video was generated using the bright-field images to show the morphology changes. For cell volume changes, PSCNPs of different concentrations (52.5, 105, and 160 μg/mL) were incubated with PC-3 cells and individual cell volume (n=5000, measured in pixels) at different time points were analyzed by the HCS Studio™ 2.0 Cell Analysis Software (Thermo Scientific). The 98% quantile of PBS treated cells (37500 pixels) was used as a benchmark.

TEM Images of Cells.

PC-3 cells were incubated with PSCNPs (160 μg/mL) for 0, 2, 4, or 6 h. Cell cultures were briefly rinsed with 0.1 M Cacodylate-HCl buffer with 5% sucrose (w/v, pH 7.25). The buffer was immediately poured out of the culture dish and replaced with a fixative containing 2.5% glutaraldehyde in 0.1 M Cacodylate-HCl buffer (pH 7.25). Cells were fixed for 1 h at room temperature. The fixative was removed from the culture dish and the cells were rinsed briefly with buffer and then post-fixed in buffered 2% (v/v) osmium tetroxide for 1 h at 4° C. A rubber policeman was used to detach cells from the culture dish. The samples were pipetted into Eppendorf snap-cap microcentrifuge tubes and centrifuged for 10 min to concentrate cells into a sample pellet before each of the following changes: Samples were rinsed three times in distilled water for 10 min each; dehydrated in a graded ethanol series for 10 min at each step: 25%, 50%, 75%, 95%, 100% and 100% followed by two changes of 10 min each in 100% acetone; infiltrated in acetone and Spurrs resin (Electron Microscopy Sciences) for 1 h or overnight: 75% acetone and 25% Spurrs, 50% acetone and 50% Spurrs, 75% acetone and 25% Spurrs, 100% Spurrs, 100% Spurrs. Samples were embedded in fresh 100% Spurrs resin and polymerized at 60° C. for 24 h in the Eppendorf tubes. The samples were removed from the tubes and mounted on plexiglass cylinders (Ted Pella) with Loctite super glue. The pelleted cell region was trimmed with a razor blade and sectioned using a Reichert-Jung Ultracut S ultramicrotome. Sixty nanometer thick sections were picked up on slot grids and allowed to dry down onto Formvar-coated aluminum racks. Grids were post-stained with uranyl acetate and lead citrate and viewed with a JEOL JEM 1011 transmission electron microscope operating at 80 kV with an AMT mid-mount digital camera with 3000×3000 resolution.

Plasma Membrane Potential.

The plasma membrane potential change was measured with DiBAC₄(3) (bis-(1,3-dibutylbarbituric acid) trimethine oxonol, Invitrogen. Lot No.: 14D1001). After the addition of PSCNPs at different concentrations (52.5, 105, and 160 μg/mL) and time points (30-150 min), PC-3 cells were incubated with 5 μM DiBAC₄(3) for 30 min at 37° C. The green fluorescence from DiBAC₄(3) was measured by a Cellomics® ArrayScan® VTI HCS Reader and analyzed using the HCS Studio™ 2.0 Cell Analysis Software.

Apoptosis/Necrosis Cell Death.

Apoptosis/necrosis was assessed through Annexin V/EthD-III staining by Apoptotic. Necrotic, and Healthy Cells Quantification Kit (Biotium, Cat No.: 30018). PC-3 cells (5×10⁴) were seeded on a tissue culture dish (Corning, 35 mm×10 mm) and were grown overnight. PSCNPs (160.0 μg/mL) were added to the dish. A working dye solution was made according to the manufacture's protocol. Briefly, into a 100 μL diluted binding buffer, 5 μL Hoechst 33342, 5 μL of FITC-Annexin V, and 5 μL ethidium homodimer-III (EthD-II) was added. After incubating with PSCNPs for 0, 2, 4, and 6 h, the cells were washed with PBS, and incubated with the dye working solution for 15 min. Fluorescence images were acquired on a fluorescence microscope using the DAPI channel for Hoechst 33342, the FITC channel for AnnexinV-FITC, and the TRITC channel for EthD-III.

Cathepsin B Release and Caspase-1 Activation.

The Magic Red Cathepsin B kit and the FAM-FLICA® Caspase-1 Assay kit were purchased from ImmunoChemistry Technologies, LLC (Bloomington, Minn.). PC-3 cells were seeded in a 8-well chamber slide (Nunc™ Lab-Tek™ II Chamber Slide™ System, ThermoFisher) at the density of 5×10⁴ cells per well and were cultured overnight. Then the cells were incubated with PSCNPs (160 μg/mL) or NaCl salt (160 μg/mL) for 2 h. Nigericin (20 μM) was used as a positive control (24 h incubation). The materials treated cells were stained with either Magic Red or FAM-FLICA® Caspase-1 at 37° C. following the manufacture's protocols. The cells were then fixed in a 4% paraformaldehyde PBS solution and mounted with VECTASHIELD anti-fade mounting medium containing DAPI (H-1200) (Vector Laboratories, US). Confocal images were taken at 100× magnification on a Zeiss LSM 710 Confocal Microscope.

Caspases-1 and Caspases-3/7 Activation Measured by Flow Cytometry.

For caspase-1 analysis, PC-3 cells were seeded at a density of 1×10⁶ cells per well in a 6-well plate overnight and then incubated with PSCNPs (160 μg/mL) for 1 or 6 h. The FAM-FLICA® Caspase-1 kit was used for cellular staining following the manufacturer's protocol. All the cells were collected and analyzed on a Beckman Coulter CytoFLEX system using the FITC channel. The results were analyzed with FlowJo v10 for caspase-1 activation. For caspase-1 and caspase-3/7 side-by-side comparison study, PC-3 cells were seeded at a density of 1×10⁶ cells per well in a 6-well plate and were cultured overnight. The cells were incubated with PSCNPs at 160 μg·NaCl/mL for 6 h, or at 52.5 μg/mL for 24 h. H₂O₂ (0.5 mM, 24 h incubation) and Nigericin (20 μM, 24 incubation) were used as caspase-3/7 and caspase-1 positive controls, respectively. The FAM-FLICA® Caspase-1 and FLICA 660 Caspase-3n Assay Kits (ImmunoChemistry Technologies, LLC) were used for cell staining. All the cells were collected and analyzed on a Beckman Coulter CytoFLEX system, using 488-nm excitation for caspase-1 measurement and 633-nm excitation for caspse-3/7 measurement. All the data were analyzed with FlowJo v10.

IL-1β Secretion.

PC-3 cells at a density of 1×10⁴ cells per well were seeded in a 96-well plate one day before the experiment. The cells were incubated with PSCNPs (105 or 160 μg/mL) for 6 h. NaCl salt (160 μg/mL) and Nigericin (20 μM) with 24 h incubation were studied as negative and positive control, respectively. The supernatants were collected and the IL-1β contents were quantified using R&D Systems Human IL-1beta DuoSet ELISA (Minneapolis, Minn.).

LDH Assays.

### (a) LDH Release Study

PC-3 cells were plated overnight at a density of 1×10¹ cells per well in a 96-well plate. The cells were incubated with PSCNPs in a dose of 13.2, 26.3, 52.5, 105, 160, 220, or 320 μg/mL for 6 h. PBS and NaCl salt at the same dose were used as controls. Supernatants were collected and the LDH contents were analyzed by LDH Assay Kit-WST (CT01-05, Dojindo, Japan). The results were normalized to PBS treated control cells.

### (b) Necrosis Inhibition Study

PC-3 cells were plated overnight at a density of 1×10⁴ cells per well in a 96-well plate. These cells were pre-treated with necrosis inhibitor glycine (5 mM) or capsase-1 inhibitor Ac-YVAD-cmk (30 μg/mL) for 1 h, and then incubated with PSCNPs (160 or 320 μg/mL) for 6 h. Cell without glycine or Ac-YVAD-cmk treatment were studied as controls. Supernatants were collected and the LDH contents were analyzed by LDH Assay Kit-WST (CT01-05, Dojindo, Japan). The results were normalized to PBS treated control cells.

### Discussion on Computational Simulation

### Model and Methodology

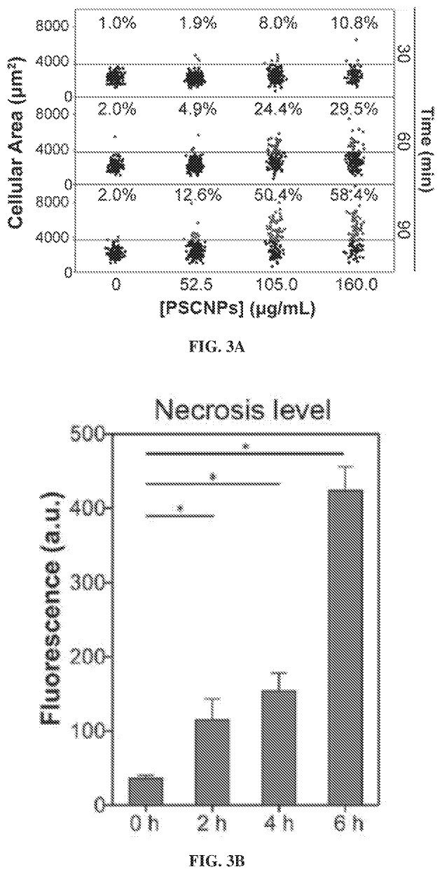

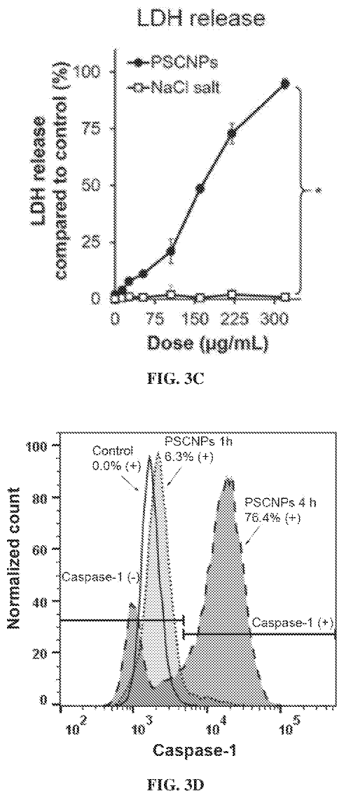

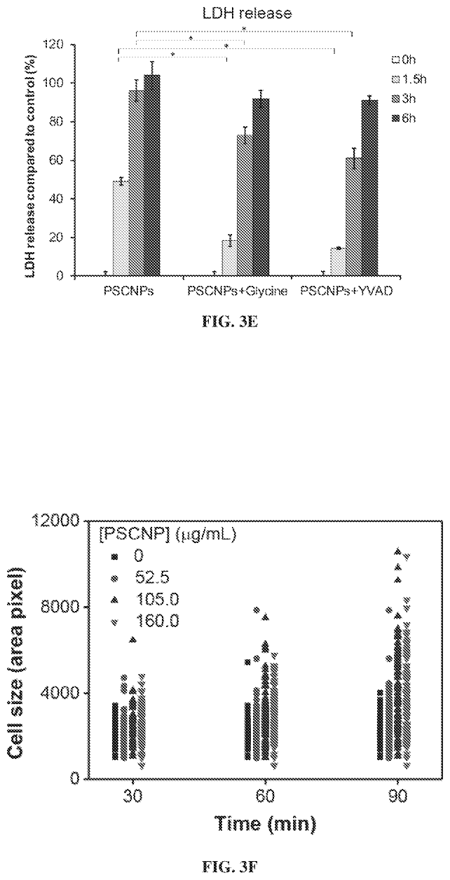

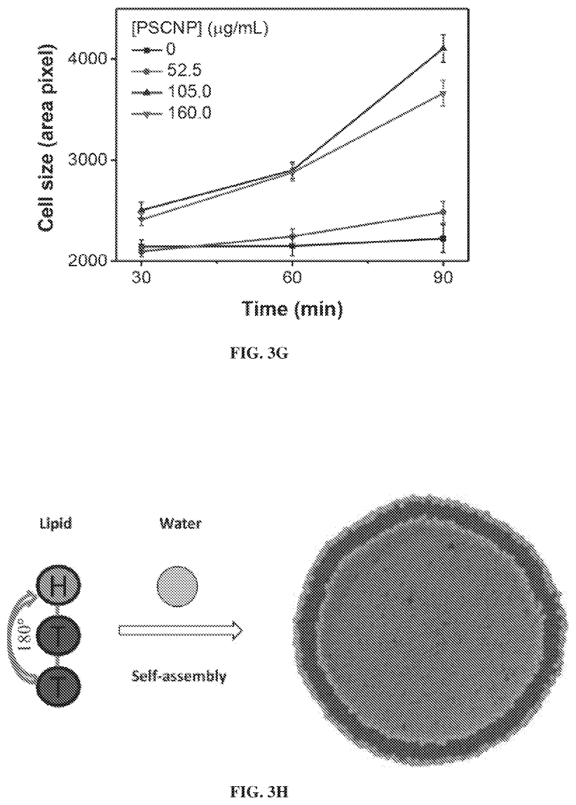

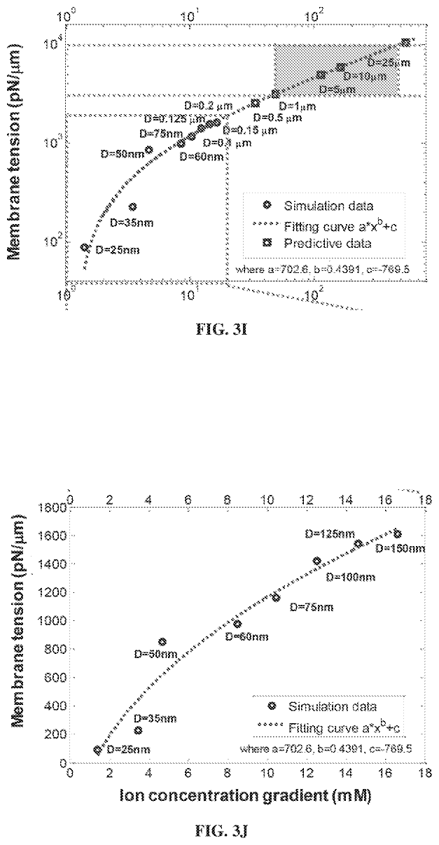

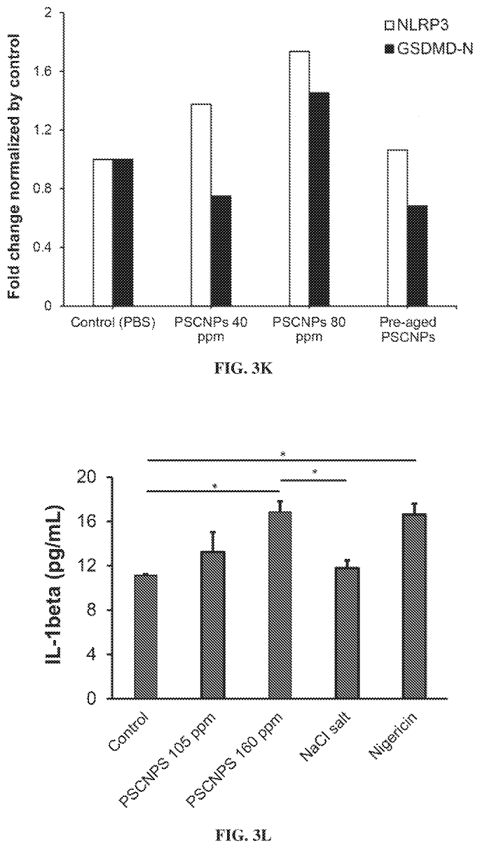

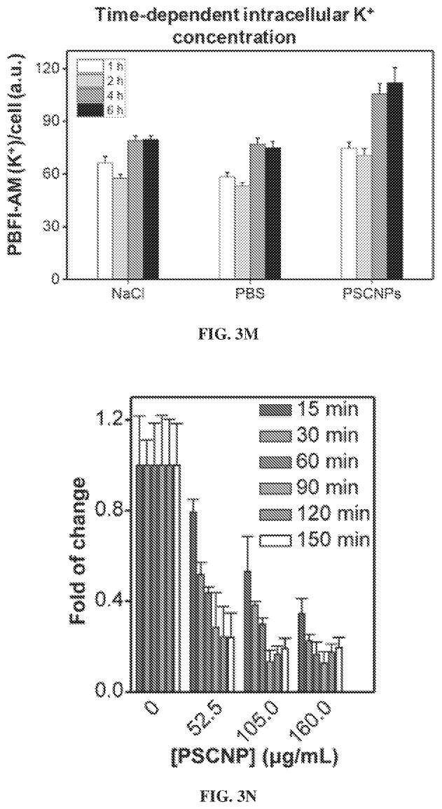

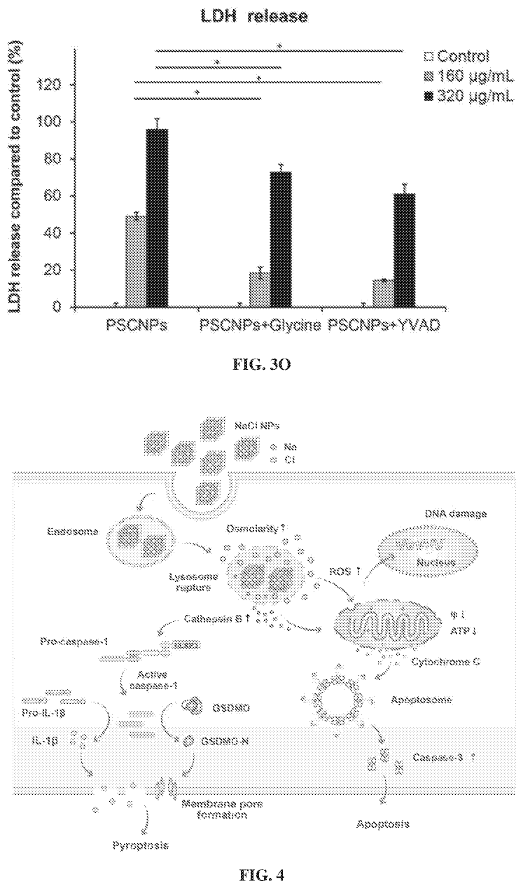

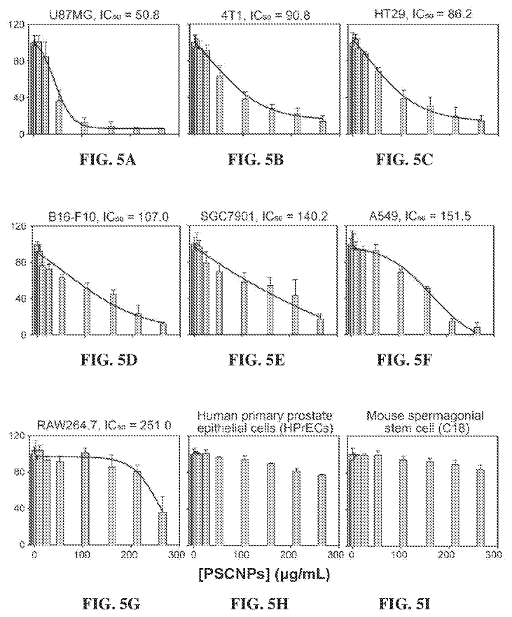

The simulation box described in FIG. 3H contained 18,000 lipid molecules that formed a spherical cell. Moreover, 289,000 water beads were included to mimic the aqueous environment. Periodic boundary conditions were applied in three directions of the simulation box. The mass, length, and time scales were an normalized in the simulations, with the unit of length taken to be σ, the unit of mass to be that of the lipid beads, and the unit of energy to be ε. All other quantities are expressed in terms of these basic units. A Velocity-Verlet algorithm was used to perform time integration, and Langevin thermostat to control the system temperature T. The integration time step is

$$Δ t = 0.01 τ \qquad ( 1 )$$

(where τ is 15 ns).

All simulations were performed using a LAMMPS package (Plimpton, J Comput Phys 117, 1-19 (1995)). The radius of the cell was 50σ. The cell as shown contained enough lipids on membrane to mimic the mechanical rupture occurring in a real cell. Similar approximation was used to study the mechanical deformation of red blood cell by Yuan et al (Fu, et al., Comput Phys Commun 210, 193-203 (2017)).

Each lipid molecule in the computational model was represented by one head bead followed by two tail beads (Cooke and Desemo, J Chem Phys 123, (2005)). The following potentials were used in the simulation to describe interactions between lipid beads:

The size of a lipid was fixed via a Weeks-Chandler-Andersen potential

$${U}_{WCA} = 4 ϵ [ {( \frac{b}{{r}_{i j}} )}^{12} - ( {( \frac{b}{{r}_{i j}} )}^{6} + \frac{1}{4} ] , {r}_{i j} < {r}_{c} = \sqrt[6]{2} b \qquad ( 2 )$$

where ∈ is the depth of the potential well, b is the finite distance at which the inter-particle potential is zero, and rᵢⱼ is the distance between the particles. In order to ensure the cylindrical lipid shape, b was set as b_{head,head}=b_{head,tail}=0.95σ and b_{tail,tail}=σ. The three beads in a single lipid were linked by two FENE bonds:

$${U}_{FENE} = {\sum }_{bonds} - \frac{1}{2} {k}_{fene} {R}_{max}^{2} ln ( 1 - \frac{{r}_{ij}^{2}}{{R}_{m α x}^{2}} ) \qquad ( 3 )$$

with the stiffness k_{fene}=30ε/σ² and the divergence length Rₘₐₓ=15σ. Lipids were straightened by a harmonic spring

$${U}_{stretching} = {\sum }_{bonds} {{k}_{stretch} ( {r}_{ij} - {r}_{o} )}^{z} \qquad ( 4 )$$

with the bending stiffness k_{stretch}=10ε/σ² and the equilibrium length r_{σ}=4σ between the head bead and the second tail bead. The hydrophobic effect was compensated by an attractive interaction between the tail beads as

$${U}_{c os} = \{ \begin{matrix}- ϵ , {r}_{ij} < {r}_{c} \\ - ϵ {cos}^{2} [ π ( {r}_{ij} - {r}_{o} ) / 2 w ] , {r}_{o} \leq {r}_{ij} \leq {r}_{o} + w \\ 0 , {r}_{ij} > {r}_{o} + w\end{matrix} \qquad ( 5 )$$

which describes an attractive potential with depth ε that smoothly tapers to zero for r>r_{c}. In this case, the decay range w was set as 1.6σ. The interaction between solvent and lipid heads in cell membrane was described by the Lennard-Jones potential function

$${U}_{LJ} = 4 ϵ [ {( \frac{b}{{r}_{i j}} )}^{12} - {( \frac{b}{{r}_{i j}} )}^{6} ] , {r}_{ij} < {r}_{o} = 2.5 σ \qquad ( 6 )$$

(where b is set as b_{water,head}=σ).

Relationship Between the Ion Concentration Gradient and Water Flux

It was assumed that the sodium and chloride concentrations were spatially uniform and the cell membrane was semipermeable to water, meaning that water particles can freely pass through the membrane. In the simulation, this process was represented by adding the water beads to the cytoplasm step-by-step. Mathematically, the osmotic pressure Π under certain sodium and chloride concentrations can be estimated using the Van't Hoff equation (Stroka et al., Cell 157, 611-623 (2014)):

$$Π = cRT \qquad ( 7 )$$

(where c is the osmolarity, R is the gas constant, and T is the absolute temperature). To simplify the problem, the hydrostatic pressure was not considered. The net chemical osmotic pressure difference Πᵢₙ−Πₒᵤₜ drives the water flux across the semipermeable membrane. Therefore, the volume of water passing through a unit area of membrane per unit time can be modeled as proportional to the chemical osmotic pressure difference

$${J}_{0} - {qV}_{w} ( {c}_{i n} - {c}_{out} ) - \frac{{qV}_{w}}{RT} ( {Π}_{i n} - {Π}_{out} ) \qquad ( 8 )$$

(where q represents the permeability rate for cells as 10⁻⁵˜10⁻⁴ m/s; V_{w} represents the molar volume of water, 18.016 mL (Stroka et al., Cell 157, 611-623 (2014)). Supposing that cells are symmetrical spheres, the total volume of water injected to the interior of a cell can be estimated as

$$V = {J}_{0} {D}^{2} {\int }_{0}^{2 π} d ϕ {\int }_{0}^{π} sin θ d θ = π {D}^{2} {J}_{0} = π {D}^{2} α ( {c}_{i n} - {c}_{out} ) RT \qquad ( 9 )$$

(where D is the cell diameter). Therefore, the concentration difference across the plasma membrane can be expressed as

$$Δ c = ( {c}_{i n} - {c}_{out} ) = \frac{V}{π {D}^{2} α RT} \qquad ( 10 )$$

The notion of membrane tension with regard to membrane rupture is widely used in cell biological literature. Based on the Law of Laplace, the membrane tension γ is directly proportional to the pressure in a cell and the radius of a cell. It can be calculated by

$$γ = \frac{p \cdot D}{4} \qquad ( 11 )$$

(p is the pressure on the membrane).
The latter can be calculated by

$$p = \frac{{σ}_{xx} + {σ}_{yy} + {σ}_{zz}}{3} \qquad ( 12 )$$

(σₓₓ, σ_{yy} and σ_{zz} is the stress).

For different size of cells, FIGS. 3I-3J shows the critical concentration gradients (Δc) upon which the plasma membrane begins to rupture (red square shadow). By curve fitting these data points, an interesting curve that has allowed us to predict the critical concentration for 25 μm cells was obtained.

### Results

Microscopic imaging found extensive PC-3 cell swelling and giant bleb formation only a few hours after incubation with PSCNPs, indicating that many cells died of necrosis rather than apoptosis. Specifically, time-lapsed imaging and pixel intensity analysis (n=5000 cells) found that the average cell area was increased by 10.8, 29.5, and 58.4% at 30, 60, and 90 min when the starting PSCNP concentration was 160.0 μg/mL (FIG. 3A, FIGS. 3F, 3G). Eventually, the inflow led to cell rupture and complete osmotic lysis. This was recorded by both live cell imaging and TEM between 4-6 h of PSCNP incubation. The cell membrane breach was also confirmed by Annexin V/EthD-III double staining and LDH assays (FIGS. 3B, 3C). Impressively, 100% LDH release was recorded when cells were incubated with 200 μg/mL PSCNPs for 6 h (FIG. 3C). To better understand the process, a coarse-grained liposome simulation model was established by a LAMMPS package (Plimpton, J Comput Phys 117, 1-19 (1995)). (FIG. 3H). The relationship between the change of concentration gradient across the plasma membrane (Δc) and the membrane tension (γ) was assessed, and used to predict the threshold at which plasma membrane starts to breach. The simulation estimates that the cell rupture will occur when Ac is in the range of 50 mM-500 mM (FIGS. 3I-3J). This agrees well with the experimental results, which detected a Ac of more than 50 mM between 4-6 h (Table 2).

TABLE 2
Time-dependent increase of intracellular ion concentrations
upon incubation with PSCNPs (160.0 μg/mL).

| Time (h) | Δ[Na⁺]ᵢₙₜ (mM)* | Δ[K⁺]ᵢₙₜ (mM)* | Δ_{C} (mM) |
|---|---|---|---|
| 0 | 0 | 0 | 0.0 |
| 1 | 0.6 | 19.4 | 20.0 |
| 2 | 3.8 | 35.9 | 39.7 |
| 4 | 13.4 | 41.8 | 55.2 |
| 6 | 8.5 | 59.3 | 68.8 |

*The concentrations were estimated by fluorescence intensity changes in FIGS. 2O and 3M. Linear response for SBH-AM (Iamshanova et al., Eur Biophys J Biophy 45, 765-777 (2016)) and PBFI-AM (Kasner and Ganz, Pbfi. Am J Physiol 262, F462-F467 (1992)) was assumed.
** The cells were incubated in an isotonic solution (Jentsch et al., Nat Rev Mol Cell Bio 17, 293-307 (2016), Armstrong, P Natl Acad Sci USA 100, 6257-6262 (2003)). Hence, Δ_{C} is equal to 0 at 0 h.
*** It is assumed that the extracellular ion concentrations remained unchanged (Jentsch et al., Nat Rev Mol Cell Bio 17, 293-307 (2016), Armstrong, P Natl Acad Sci USA 100, 6257-6262 (2003)).

However, the cell lysis was not a mere physical process; rather, it was mediated, at least in part, by pyroptosis, also known as caspase-1-dependent cell death (Labbe et al., Prog Inflamm Res Ser, 17-36 (2011), Miao et al., Rev 243, 206-214 (2011), Schroder, et al., Cell, 140, 821 (2010)). Pyroptosis is a form of programed necrosis, and is characteristic of inflammasome induction, pro-inflammatory cytokine release, and caspase-1 activation (Man et al., Immunol. Rev. 277, 61-75 (2017)). The activated caspase-1 promotes the release of the N-terminal of gasdermin-D (GSDMD), which translocates to the plasma membrane and perforates it, causing water inflow (Liu, X., et al. Nature 535, 153-158 (2016), Shi et al., Nature 526, 660-665 (2015)). PSCNP treated cells had significantly increased caspase-1 activity by FAM-FLICA caspase-1 staining. Flow cytometry showed that the caspase-1 activity was increased by 76.4% at 4 h incubation with PSCNPs (160 μg/mL, FIG. 3D). The impact of two necrosis inhibitors, glycine and Ac-YVAD-cmk peptide, were also assessed. While glycine is a general necrosis inhibitor (Weinberg et al., Cell. Mol. Life Sci. 73, 2285-2308 (2016)), Ac-YVAD-cmk selectively inhibits the activation of caspase-1 (Zhang et al., Sci Rep-Uk, 6 24166 (2016)). Both agents were effective at suppressing cell lysis, reducing LDH release by 72.9% and 60.9%, respectively (FIG. 3E, 3O). The activation of pyroptosis pathway was also confirmed by NLRP3 inflammasome induction, GSDMD N-terminal fragment release (FIG. 3K), and elevated IL-1β secretion (FIG. 3L).

Conventionally, pyroptosis is observed in immune cells upon the detection of pathogen infection by toll-like receptors (TLR) or NOD-like receptors (NLRs) (Bergsbaken et al., Microbiol. 7, 99-109 (2009), Bortoluci and Medzhitov, Cell. Mol. Life Sci. 67, 1643-1651 (2010)). How PSCNPs trigger caspase-1 activation in cancer cells is unknown. One possibility is that the osmotic pressure induced by PSCNPs causes endosomes/lysosomes to rupture, leading to the release of cathepsin B to the cytosol (Szabo and Csak, Journal of Hepatology 57, 642-654 (2012)). Cathepsin B induces the formation of NLRP3 inflammasomes (Mirshafiee et al., Acs Nano 12, 3836-3852 (2018)), which in turn activates caspase-1. This model is supported by Magic Red staining, which found a diffusive distribution pattern of cathepsin B in PSCNP treated cells (as opposed to a punctate distribution in untreated cells). Moreover, time-relapsed cell imaging recorded a reduced level of LysoTracker positive staining in PSCNP treated cells, also indicating endosome/lysosome rupture. Another possibility is that caspase-1 activation is triggered by K⁺ efflux. This is based on the observation that in addition to Na⁺ and Cl⁻, the intracellular K⁺ level was also elevated after incubation with PSCNPs (FIG. 3M), possibly as a result of Na⁺/K⁺ pump activities in response to an increased Na⁺ concentration. This would further exacerbate potassium charge separation, leading to a hyperpolarized plasma membrane, which was supported by DiBAC₄ staining results (FIG. 3N). The enhanced potassium gradient will facilitate K⁺ efflux, a known trigger of pyroptosis (Munoz-Planillo et al., Immunity 38, 1142-1153 (2013), Bergsbaken et al., Nat Rev Microbiol 7, 99-109 (2009)). Notably, mitochondria breach and cytochrome c release does not occur in conventional pyroptosis (Jesenberger et al., J Exp Med 192, 1035-1045 (2000), Cervantes et al., Cell Microbiol. 10, 41-52 (2008)). This indicates that PSCNP treatment activates both apoptosis and pyroptosis pathways (FIG. 4): at high PSCNP doses and early time points, cells mainly die of caspase-1-dependent pyroptosis, whereas at low doses and longer time points, cells die of caspase-3-dependent apoptosis due to cumulative oxidative stress and DNA/lipid damage.

### Example 5: The Killing Effect of NaCl Nanoparticles on Cancer Cells Versus Normal Cells

### Materials and Methods

Intracellular Sodium Contents.

A panel of cell lines, including 4T1, HT29, A549, SGC7901, PC-3, U-87 MG, B16-F10. RAW264.7, HPrECs and C18-4 cells, were cultured in 75 cm² Corning cell culture flasks in a humidified, 5% carbon dioxide atmosphere at 37° C. Cells were collected when they reached 85% confluency and the cell numbers were counted using a hemocytometer. After centrifugation (1200 rpm, 5 min), the cell pellets were washed with 5 mL Na⁺-free HEPES buffer three times. The final cell pellets were suspended in D.I. water and homogenized by probe sonication. The intracellular sodium concentration [Na⁺]ᵢₙₜ was measured using a Na⁺ electrode (HORIBA LAQUAtwin Na-11). The results were normalized to cell numbers to obtain intracellular sodium content ([Na⁺]ᵢₙₜ) for each cell line.

Cellular Uptake of NPs

A panel of cell lines, including T24, UMUC2, K1970 and HPrEC were cultured in 6-well plate in a humidified, 5% carbon dioxide atmosphere at 37° C. Rhod-PE labeled NaCl NPs at 200 μg/ml were incubated with each cell line for 2 h. Cells were collected to run flow cytometry.

### Results

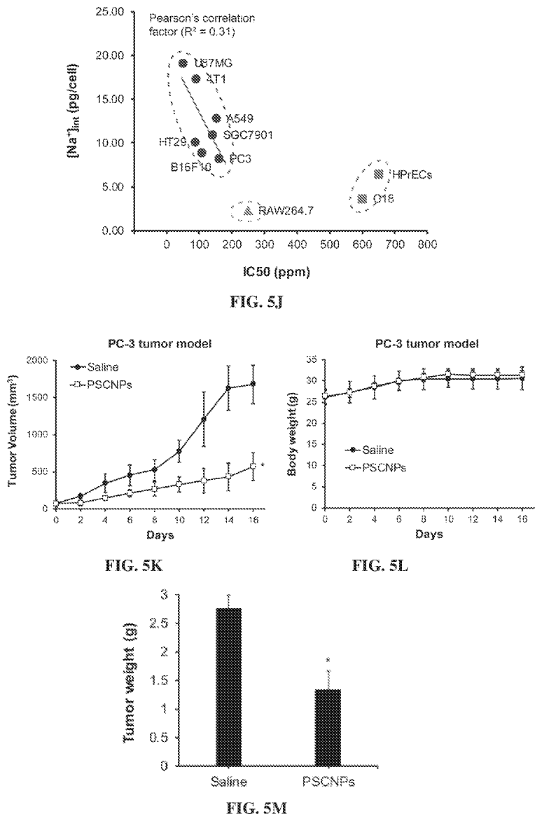

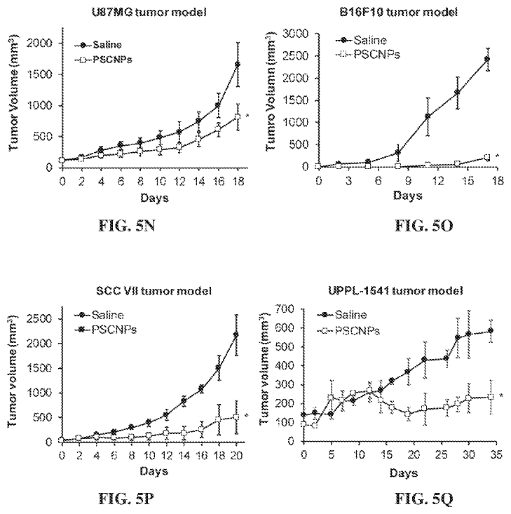

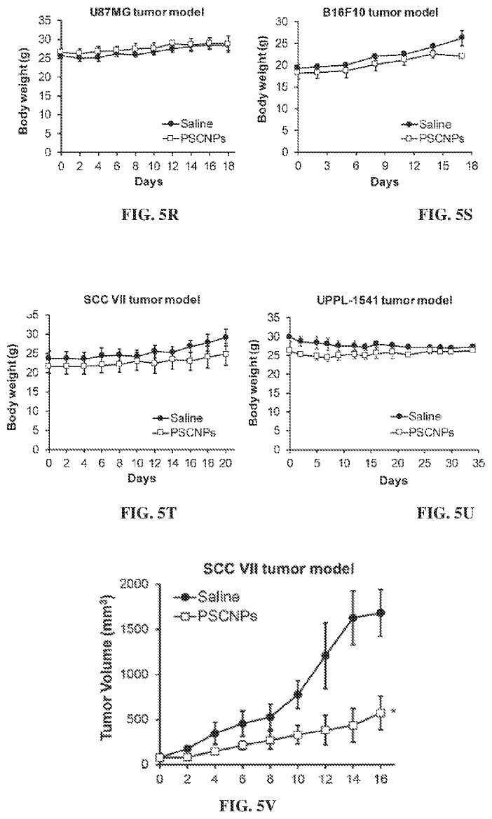

The cytotoxicity of PSCNPs was also examined with a panel of other cell lines (FIGS. 5A-5I). The viability of normal cells such as HPrECs (human primary prostate epithelial cell line) and C18-4 (mouse spermatogonial stem cell) was minimally affected in the tested concentration range (3.25 to 320 μg/mL, FIGS. 5A-5I). As a comparison, all cancer cells were effectively killed by PSCNPs, with IC₅₀ values ranging from 50 to 160 μg/mL (FIGS. 5A-5I). This selective toxicity is intriguing. One reason is that fast proliferating cells tend to take up more nanoparticles (Chaves et al., Int J Nanomed 12, 5511-5523 (2017)). But this does not explain why RAW264.7 cells, a phagocytic macrophage cell line, were also relatively resistant to PSCNPs (FIGS. 5A-5I). Another plausible factor is that cancer cells possess high intracellular sodium concentrations ([Na⁺]ᵢₙₜ), making them inherently more susceptible to an osmotic shock. In the 70s', Cone et al. proposed that an elevated [Na⁺]ᵢₙₜ and a relatively depolarized plasma membrane are characteristics of cancer cells (Cone, Journal of theoretical biology 30, 151-181 (1971). Cone, Ann N Y Acad Sci 238, 420-435 (1974). Cone and Cone, Science 192, 155-158 (1976)).

This was confirmed by the follow-up elemental analysis studies (Cameron et al., Cancer Res 40, 1493-1500 (1980)), with some reporting that the [Na⁺]ᵢₙ/[K⁺]ᵢₙₜ ratio in cancer cells could be 5 times higher than in normal cells (Zsagy et al., J Cell Biol 90, 769-777 (1981)). Indeed, all of the tested cancer cells show a higher [Na⁺]ᵢₙₜ than macrophages and primary cells (FIG. 5J). K-means clustering clearly reveals the difference between cancer cells and primary cells with regard to cytotoxicity and its correlation with cells' [Na⁺]ᵢₙₜ. Among cancer cells, there is a moderate correlation between [Na⁺]ᵢₙₜ and IC₅₀, with a Pearson correlation coefficient R² of 0.31 (FIG. 5J). These results support [Na⁺]ᵢₙₜ as an important factor behind the sensitivity of cancer cells to PSCNP treatment. It is believed that cancer cells adopt a high [Na⁺]ᵢₙₜ as an anti-apoptosis measure (apoptosis is characteristic of cell shrinkage), which makes them intrinsically susceptible to PSCNPs induced cell necrosis.

Flow cytometry studies on cellular uptake found significantly elevated PSCNPs uptake by bladder cancer cells relative to normal epithelial cells including normal urothelial cells (FIG. 2R).

### Example 6: NaCl Nanoparticles are Cancer Therapeutics

### Materials and Methods

In Vivo Therapy Study.

Animal studied were performed according to a protocol approved by the Institutional Animal Care and Use Committee (IACUC) of the University of Georgia. The animals were maintained under pathogen-free conditions. PC-3 tumor model was generated by subcutaneously injecting 2×10⁶ cells in 50 μL PBS into the right flank of 5-6 week old male athymic nude mice (Charles River). U-87 MG tumor model was generated in female athymic nude mice (Charles River) following the same method as PC-3 model. B16F10 tumor model was generated by subcutaneously injecting 2×10⁵ cells in 50 μL PBS into the right flank of 5-6 week old female C57BU6 mice (Charles River). SCC VII tumor model was generated by subcutaneously injecting 2×10¹ cells in 50 μL PBS into the right flank of 5-6 week old female C3H/HeN mice (Charles River). UPPL-1541 tumor model was generated by subcutaneously injecting 1×10⁶ cells in 50 μL PBS into the right flank of 5-6 week old female C57BL6 mice (Charles River).

For therapy studies, PC-3 tumor bearing mice were randomly divided into 2 groups (n=5 for each group). When the average tumor volume was about 100 mm³, PSCNPs (9 mg/mL, 50 μL) were intratumorally injected on day 0, 2 and 4. For control, saline at the same volume was injected. For both PSCNPs and saline, the injection was performed at five sites of the tumor to ensure good coverage. The tumor size and body weight were inspected every two days. The tumor was measured in two dimensions with a caliper, and the tumor volume was estimated as (length)×(width) 2/2. U-87 MG tumor model followed the same therapy method as PC-3 tumor model. B16F10, SCC VII tumor models were treated with PSCNPs when the average tumor volume was about 40 mm³, while UPPL-1541 tumor model was treated at 100 mm³. PSCNPs (27 mg/mL, 50 μL) were intratumorally injected on day 0, while saline at the same volume was injected as control group. The tumor size and body weight measurements were the same as PC-3 model. At the end of the PC-3 tumor therapy experiment, autopsies were performed. The tumor were dissected for morphological and histological examination. In particular, these tissues were sectioned into 4 μm slices for H&E, TUNEL staining (in situ Apoptosis Detection Kit, ab206386, Abcam, US) and caspase-1 staining. The caspase-1 IHC staining kit was purchased from Abcam, US. The kit includes anti-caspase-1 antibody (ab1872), goat anti-rabbit IgG H&L (HRP) (ab6721), rabbit specific HRP/DAB (ABC) detection IHC kit (ab64261), and methyl green pyronin (RNA DNA Stain) (ab150676). All the staining followed the manufacturing protocols.

### Results

PSCNPs were tested as a tumor ablation method in vivo. Unlike conventional chemotherapy, the toxicity of PSCNPs is temporal: they induce fast and lethal damage to cancer cells, and then reduce to completely benign NaCl salts, causing no chronic or systemic toxicities. To investigate, a subcutaneous tumor model established with PC3 cells was established. PC-3 cells were introduced to the right flank of male nude mice (n=5). When the tumor size reached 100 mm³, PSCNPs (50 μL, 9 mg/mL) were intratumorally (i.t.) injected to the animals every other day for 3 total injections. For control, NaCl saline (9 mg/mL) was i.t. injected at the same NaCl dose. Relative to the control, PSCNP treatment suppressed tumor growth by 66% on Day 16 (FIGS. 5K, 5M). Post-mortem hematoxylin/eosin (H&E) staining exhibited large areas of nuclear shrinkage and fragmentation in tumor tissues. Moreover, both TUNEL and anti-caspase-1 assays found extensive positive staining in PSCNP treated tumors, indicating cell death through both apoptosis and pyroptosis, which is consistent with the in vitro observations. Meanwhile, no body weight drop was detected throughout the study (FIG. 5L) and no sign of toxicity was found in major organs. Similar treatment outcomes were observed with other tumor models, including U87MG (human glioblastoma), B16F10 (mouse melanoma), SCC VII (mouse head and neck squamous carcinoma), and UPPL-1541 (mouse bladder cancer) (FIGS. 5N-5U).

### Example 7: NaCl Nanoparticles Induce Release of ATP, HMGB-1, and Expression of CRT

### Materials and Methods

CRT Expression on Cell Surface Inflow Cytometry Assessment.

CRT expression on cell surface in flow cytometry assessment. T24, UMUC2, UPPL-1541, BBN963, B16F10, and SCC VII cells are seeded into the 6 wells plate at 1×10⁶ per well. After overnight incubation, the cells were treated with NaCl particles (PSCNPs) (160 μg/mL) for 2 h. PBS treated cells were used as a control. All the cells were collected by cell lifters, and incubated with an Alexa Fluor® 647-conjugated anti-CRT antibody (ab196159, 1/500. Abcam) for 30 min at 4° C. The cells were incubated in 500 μL PBS containing 50 μg/mL propidium iodide before washing and assessment on a flow cytometer. The data were expressed in histogram compared to the PBS treated control cells.

ATP and HMGB-1 Release.

Cells were seeded into 96-well plates at the density of 1×10⁴ cells per well and incubated overnight. Then the cells were treated with PSCNPs dispersed in PBS at a dose range of 13.2-320 μg/mL for 1, 2, 4 h and 24 h. Cell supernatant was collected after 1-4 h incubation and tested in ATP 1step Luminescence Assay System, 100 mL ATP Assay Kit (PerkinElmer, US) following the manufacture's protocol. A 10-fold serial dilution series of ATP in culture medium (1 μM to 1 μM) were created to build up a standard curve and calculate the absolute amount of ATP in the supernatant. The luminescence was measured by a microplate reader (Synergy Mx, BioTeK). All measurements were performed in sextuplicate. Cell supernatant was collected after 24 h incubation and tested in an ELISA kit (IBL International GmbH), according to the manufacturer's instructions. NaCl salt and PBS were used as controls.

### Results

One interesting observation is that overall, much better treatment outcomes were seen in syngeneic tumor models (UPPL-1541. B16F10 and SCC VII) than xenograft tumor models (PC-3, U87MG). Taking SCC VII tumors for instance, 20% of the mice became tumor free after PSCNP treatment and survived for more than 8 months (FIG. 5W). These results indicate that in immunocompetent mice, PSCNPs may not only kill cancer cells, but also stimulate an anticancer immunity. Necrosis is a highly immunogenic process (Inoue and Tani, Cell Death Differ., 21, 39 (2014), Zhang, et al., J. Han, Cell Res., 28, 9 (2018)).

In addition, it was observed that cancer cells succumbing to PSCNPs showed increased surface presentation of calreticulin (CRT) (FIGS. 6E and 6F), as well as elevated secretion of adenosine triphosphate (ATP) (FIG. 6A), and high mobility group box 1 (HMGB-1) (FIG. 6B), all of which are established hallmarks of immunogenic cell death or ICD (Kroemer, et al., Annu. Rev. Immunol., 31, 51 (2013)).

FIGS. 6E and 6F are histograms of CRT presentation on dying B16F10 and SCC VII cells. Cells were treated with 160 μg mL-1 PSCNPs for 2 h. FIGS. 6A and 6B show time- and dose-dependent ATP release from B16F10 and SCC VII cells treated by PSCNPs (13.2-320 μg mL-1; *p<0.05) for 1, 2, and 4 h.

NaCl NPs treatment induced a significantly increased secretion of ATP in bladder cancer cell lines (FIGS. 12A-12D) and elevated CRT presentation (FIGS. 12E, 6E) in dying cancer cells in both bladder cancer cell lines (FIG. 12E) and B16F10 cells (FIG. 6E).

FIGS. 6C and 6D show HMGB-1 release from B16F10 and SCC VII cells after PSCNP treatment (13.2-320 μg mL-1) at 24 h. NaCl salt and PBS were studied as controls.

Reduced HMGB-1 secretion at very high concentration were due to extensive cell death at 24 h. (*p<0.05 compared to PBS treated control cells) It was noted from previous studies that CRT, HMGB-1 and ATP can bind to pattern recognition receptors (PRRs) on dendritic cells (DCs) (e.g., CD91 (Pawaria and Binder, Nat. Commun. 2, 521 (2011), Berwin, et al., EMBO J. 22, 6127 (2003). Gardner and Ruffell, Trends Immunol., 37, 855 (2016)) and SR-A (Berwin, et al., EMBO J. 22, 6127 (2003), Hu, et al., Biochem. Biophys. Res. Commun., 392, 329 (2010)) for CRT, RAGE, TLR2/4/9 for HMGB-1 (Inoue and Tani, Cell Death Differ., 21, 39 (2014)) and P2RX7/P2RY2 for ATP (Inoue and Tani, Cell Death Differ., 21, 39 (2014), Gardner and Ruffell, Trends Immunol., 37, 855 (2016)). This promotes DC migration, maturation and antigen cross presentation to T cells, and in turn boosts cellular immunity against tumors (Gardner and Ruffell, Trends Immunol., 37, 855 (2016). McDonnell, et al., Clin. Dev. Immunol., 2010, 539519 (2010)).

### Example 8: NaCl Nanoparticles Induce a Vaccination Response to Cancer

### Materials and Methods

In Vivo Vaccination Approach to Induce Immune Response.

Animal studied were performed according to a protocol approved by the Institutional Animal Care and Use Committee (IACUC) of the University of Georgia. A timeline for the vaccination schedule is described in FIGS. 7A and 7C. B16F10 cells were exposed to PBS, 320 μg/mL NaCl NPs for 6 h, as well as F/T method to induce ICD. The dying 2×10⁵ B16F10 cells were injected into the right flank of 5-6 week old female C57BL6 mice (Charles River) (n=5). 6 days after the injection, the animals received SC injection of viable B16F10 cells 2×10⁵ in the contralateral (left) flank. Similar as B16F10 cells, SCC VII cells were exposed to PBS and 320 μg/mL NaCl NPs for 24 h to induce IDC biomarkers release. The dying 2×10⁵ SCC cells were injected twice into the right flank of 5-6 week old female C3H/HeN mice (Charles River) (n=5), 6 days apart. 12 days after the injection, the animals received SC injection of viable SCC cells 2×10⁵ in the contralateral (left) flank. Tumor size was measured by a digital caliper every 2-3 days. The tumor volume was calculated according to the formula (length)×(width) 2/2. Animals were sacrificed on Day 22 for B16F10 tumor model and on Day 24 for SCC VII tumor model. Tumors were collected for flow cytometry analysis.

In Situ Vaccination and Cancer Therapy in SCC VII Bilateral Tumor Model

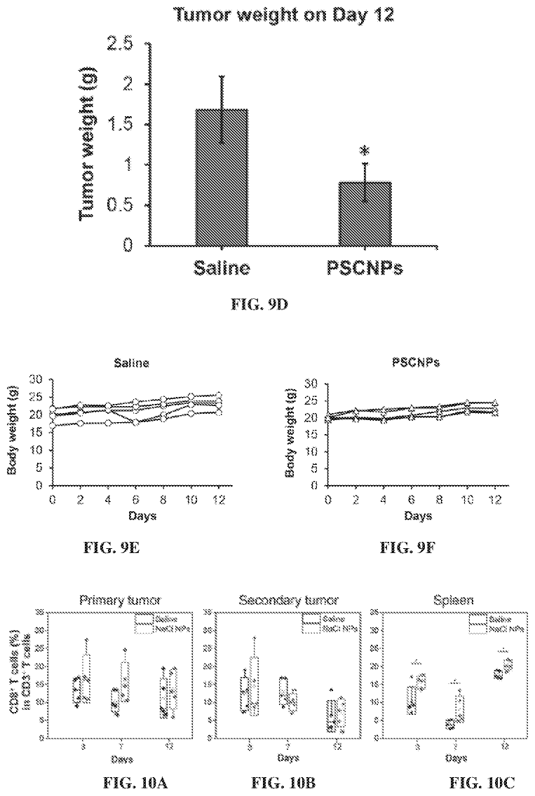

Time lines for vaccination schedules are described in FIGS. 8A and 9A. Cells were mixed with Matrigel for tumor inoculation. SCC VII bilateral tumor model was created by SC injecting 1×10⁶ SCC cells into the right flank as the primary tumor and 0.5×10⁶ SCC cells in the left flank as the secondary tumor of 5-6 week old female C3H/HeN mice (Charles River) (n=5). 12 days after the injection, the animals received one time NaCl NPs treatment. Each mouse in NPs group was injected 1.35 mg NaCl NPs in 50 μL saline. Saline treated group was used as a negative control. Tumor bearing mice w/o treatment were used as an untreated control. The tumor volume was calculated according to the formula (length)×(width) 2/2. On Day 12, primary and secondary tumors, spleen, PBMCs and TDLNs were collected after euthanizing the animal to conduct flow cytometry study.

Flow Cytometry Analysis

The tumor pieces obtained for single-cell analysis were cut into smaller pieces with scissors and digested in DMEM with 0.5 mg/mL collagenase type I (Worthington Biochemical Corporation) at 37° C. for 1 h. The digested tissues were gently meshed though a 70 μM cell strainer, twice. Red blood cells were lysed by Ack lysing buffer (Gibco) according to the manufacturer's instructions. The single-cell suspensions were washed twice and resuspended in staining buffer. Following cell counting and aliquoting, the suspensions were incubated with FcBlock (TruStain fcX™ anti-mouse CD16/32, clone 93, BioLegend) for 20 min to avoid nonspecific binding. Staining was then performed by using various combinations of fluorophore-conjugated antibodies for 40 min at 4° C. The following anti-mouse antibodies were purchased from BD Biosciences: CD45-APC-Cy7 (#557659, 1/100), CD45-V450 (#560501), CD4-BV605 (#563151, 1/100), CD8α-PE (#561095, 1/100), CD8α-FITC (#563030, 1/100), CD11c-V450 (#560521, 1/100), CD86-BV605 (#563055, 1/100), CD80-PerCP-Cy5.5 (#560526, 1/100), CD11b-PE (#553311, 1/100). Foxp3-PE (#60-5773, 1/100), live/dead cell assay Ghost Red 710 (#13-0871, 1/100) were purchased from TONBO biosciences. IFN-γ-APC (#505810, 1/100), CD25-PerCP-Cy5.5 (#102030, 1/100) and CD3-APC-Cy7 (#100222, 1/100) were purchased from BioLegend. Multi-parameter staining was used to identify the following populations of interest: (a) CD8+ T cells (CD45+CD3+CD8+CD25+), (b) Tregs (CD45+CD3+CD4+Foxp3+), (c) DCs (CD45+CD11c+), (d) CD86+ DCs (CD45+CD11c+CD86+), (e) CD80+CD86+ DCs, (f) CCR7+ DCs (CD45+CD11c+CD80+CD86+CCR7+), (f) CD8+ DCs (CD45+CD11c+CD8+CD11b−). For intracellular Foxp3 and IFN-γ staining, cells were further fixed and permeabilized using a Foxp3/Transcription Factor Staining Buffer Set (eBioscience). After washing, cells were used for flow cytometry analysis (CytoFLEX, Backman Coulter). The data were processed by FlowJo 10.0. Doublets were excluded based on forward and side scatter. Dead cells were excluded based on negative signal of Ghost Red 710 staining.

### Results

B16F10 cells were killed by either PSCNPs or freeze thaw (F/Z) treatment (a common method in vaccine preparation), and subcutaneously inoculated the dead cells to healthy C57BL/6 mice. On day 7, live B16F10 cells were injected to the contralateral flank of the animals. PSCNPs treatment compared to Saline treated mice, and conventional F/T method is illustrated in FIG. 7B. Similarly, PSCNPs treatment in anti-SCC tumor vaccination showed more than 96% inhibition of tumor growth than the non-vaccinated mice, and enhanced T cell response, including 1.07 fold increase of CD8+T cells, 0.68 fold decrease of Treg, 1.57 fold increase of CD8+ T cells/Treg ratio, 1.34 fold increase of DCs, 1.11 fold increase of activated CD86+ DCs, as well as 1.29 fold increase of antigen presenting CD8+DCs (FIG. 7D, Table 3).

TABLE 3
After studies in FIGS. 7C-7D, animals were euthanized and
tumors were collected for flow cytometry analysis. The relative
frequency of CD8 + T cells, Tregs (CD4 + Foxp3+ T cells), DCs,
CD86 + DCs, and CD8 + DCs, as well as CD8+/Tregs ratio,
were examined.

| | Control mice w/o | NaCl NPs |
|---|---|---|
| | vaccination | vaccinated mice |
| CD8 + T cells | 1 | 1.07 |
| Treg | 1 | 0.68 |
| CD8+/Treg ratio | 1 | 1.57 |
| DCs | 1 | 1.34 |
| CD86 + D | 1 | 1.11 |
| CD8 + DCs | 1 | 1.29 |

The results in Table 3 indicate strong T cell responses after NaCl NPs vaccination. The data were collected using Flow Cytometry to determine CD8+ T cells, Treg (CD4+Foxp3+ T cells), CD8/Tregs ratio, CD86+ DCs and CD8+ DCs in FlowJo 10.0 and normalized based on control group, which was considered as 1 for each subset. Mice vaccinated with PSCNP-killed cancer cells showed much greater resistance to a subsequent live cancer cell challenge, with all animals remaining tumor-free for more than 2 weeks (FIG. 7B). Similar results were observed with SCC VII cells in C3H mice (FIG. 7D).

Another study in a SCC bilateral tumor model showed that PSCNP treatment slowed down 48% secondary tumor growth (FIG. 8B) compared to Saline group. PSCNPs stimulated the immune response by upregulating CD8+ T cells, reduce Treg, increasing CD8+/Treg ratio, and activating DCs. Specifically, PSCNP in situ vaccination increased CD8+ T cells more than 1.13 for all the collected tissues, increased activated CD8+IFN-γ T cells over 1.02 and reduced Treg more than 0.65 fold within tumors and spleen, caused CD8+/Treg ratio 16.92 fold increase in the secondary tumor. For DCs, PSCNP killing effect in cancer cells induced more than 1.6 fold increase of DCs in the primary tumor and TDLNs, enhanced DCs co-stimulation (CD86+DCs and CD80+CD86+DCs) for almost all the collected tissues and stimulated DCs homing to TDLNs more than 1.3 fold change in tumors and spleen. Collectively, NaCl NP-treatment to the primary tumor can serve as an in situ vaccine to kill cancer cells and release DMAPs to recruit DCs. DCs uptake the dying tumor cells, home to the TDLNs, present the neoantigens on the surface through cross-presentation to T cells. Thus results indicate that the activated CD8+ T cells can further infiltrate to the tumor site and kill the cancer cells in the secondary tumor.

Table 4 shows fold changes of T lymphocyte and DC subsets in different tissues compared to saline-treated group at Day 12 post-treatment. Primary and secondary tumors, spleen. PBMCs and TDLs were collected after euthanizing the animal to conduct flow cytometry study. Data were analyzed by FlowJo 10.0 and normalized based on saline- treated group, which was considered as 1 for each subset.

| | | Primary tumor | Secondary tumor | Spleen | Blood | TDLNs |
|---|---|---|---|---|---|---|
| T cell | CD8+ | 1.22 | 1.14 | 1.15 | 1.13 | 1.14 |
| | CD8+IFNr+ | 1.02 | 1.07 | 1.06 | 0.73 | 0.86 |
| | Treg (CD4+Foxp3+) | 0.26 | 0.07 | 0.65 | 1.02 | 1.19 |
| | CD8+/Treg ratio | 4.75 | 16.92 | 1.77 | 1.11 | 1.06 |
| DCs | CD11C+ DCs | 1.60 | 0.98 | 0.66 | 0.91 | 1.85 |
| | CD86+ DCs | 1.32 | 2.28 | 1.19 | 1.07 | 0.93 |
| | CD80+CD86+ DCs | 1.85 | 1.49 | 0.97 | 1.22 | 1.81 |
| | CCR7+ DCs | 1.54 | 1.30 | 1.57 | 0.68 | 1.00 |

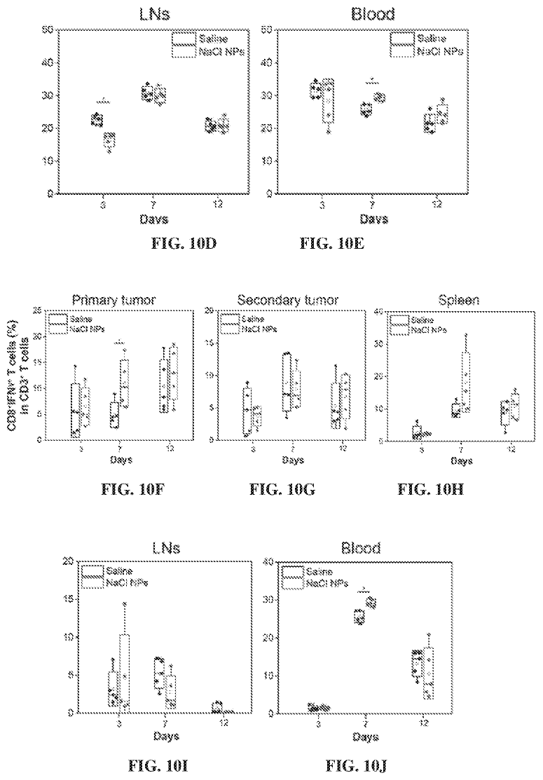

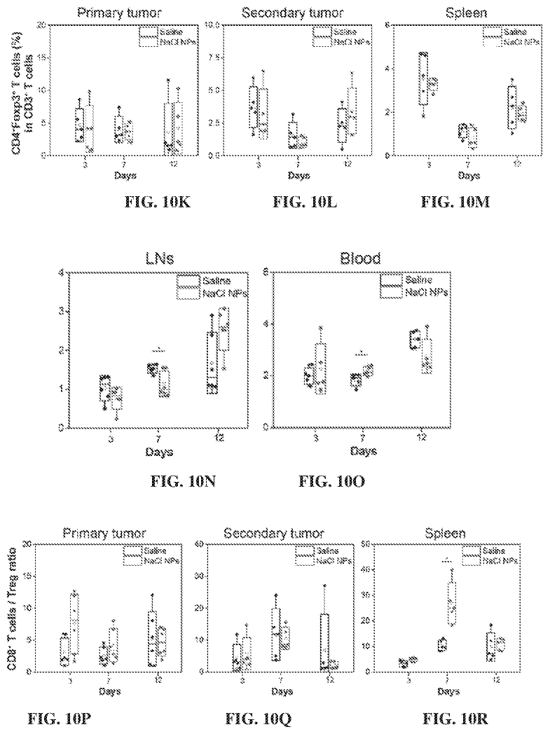

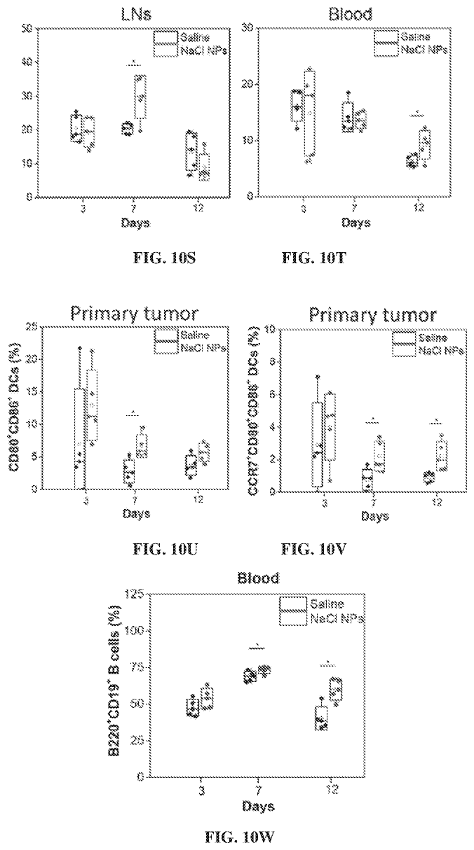

PSCNPs or saline were intratumorally injected into the primary tumors, but left the contralateral tumor (secondary tumor) untreated (FIG. 9A). Results show that the secondary tumors in the PSCNP group grew at a much lower speed than the saline control, showing a tumor inhibition rate of 53% on day 12 (FIGS. 9B-9D). Meanwhile, there was no body weight drop throughout the study (FIGS. 9E-9F). In a separate study, euthanized animals on day 3, 7, and 12 post particle/saline injection, harvested tumors, spleen, blood, and tumor-draining lymph nodes (TDLNs), and analyzed leucocyte profiles by flow cytometry. Relative to the saline control, PSCNP injection led to increased CD8+ T cell frequencies, which was the most significant in the spleen samples at all three time points (FIGS. 10A-10E). In particular, effector T cell (CD8+IFN-γ+) population was increased in the primary tumor and blood on day 7 (FIGS. 10F-10J). The CD8+/Treg (CD4+Foxp3+) ratio was also increased in the primary tumor, spleen, TDLNs, and blood on day 7 and 12 (FIGS. 10P-10T). Blood B cell (B220+CD19+) frequency was also elevated relative to the saline control on day 7 and 12, indicating the possibility of enhanced humoral immunity (FIG. 10W). One factor behind the boosted adaptive immune response was ICD promoted DC infiltration and maturation (Gardner and Ruffell, Trends Immunol., 37, 855 (2016)). Increased numbers of activated DCs (CD80+CD86+) and TDLN-homing DCs (CD80+CD86+CCR7+) were observed in the primary tumors on day 7 and 12 (FIGS. 10U-10V). Collectively, the results indicate that PSCNPs killed cancer cells and converted the dying cancer cells to an in situ vaccine. It was noted that the treatment did not lead to significant increase of CD8/Treg ratios in the secondary tumors.

### Example 9: NaCl Nanoparticles Used in Combination with αPD-1 for Tumor Suppression

### Materials and Methods

BBN bilateral tumor model was created by subcutaneously injecting 2×10⁶ BBN963 cells into the right flank as the primary tumor and 0.7×10⁶ SCC cells in the left flank as the secondary tumor of 5-6 week old female C57BL6 mice. 21 days after the injection, the animals received NaCl NPs treatment 3 times every 3 days. Each mouse in NPs group was injected 3.25 mg NaCl NPs in 50 μL saline. Saline treated group (50 μL) was used as a negative control. PSCNPs (i.t.) and anti-PD-1 antibodies co-administration was used for combination therapy. The tumor volume was calculated according to the formula (length)×(width) 2/2.

### Results

The combination therapy showed more effective tumor suppression than PSCNPs or αPD-1 alone (FIGS. 11A-1B). FIG. 11A is a tumor growth curves showing PSCNPs+αPD-1 induced most efficient tumor growth suppression, with 77.8% animals remaining tumor-free on Day 65. FIG. 11B is a plot of body weight changes. No body weight drop or signs of systemic toxicity were observed throughout the experiment.

Collective, results in Examples 7 and 8 show that NaCl NPs induce immunogenic cell death (ICD) in B16F10 and SCC VII head neck cancer cell lines both in vitro and in vivo (FIGS. 6A-6D, 7A-7D. Table 3). NaCl NPs treatment induces the release of ICD biomarkers, such as ATP and HMGB-1, in both B16F10 and SCC VII cell lines, compared to negligible impact of NaCl salt and PBS control. Anti-B16F10 vaccination approach by injecting NaCl NP-treated dying B16F10 cells causes stronger ICD response than conventional freeze thaw (FIT) method. Similar approach in anti-SCC VII study also shows the ICD effect induced by NaCl NPs treated dying SCC Vii cells. NaCl NPs vaccination stimulates strong T cell responses compared to non-vaccinated group, including the reduction of Treg cells (CD4+Foxp3+ T cells), increase of CD8+ T cells and CD8+/Treg ratio. NaCl NPs vaccination also boosts DCs activation compared to non-vaccinated group, such as increasing CD86 costimulator expression in CD86+ DCs subsets and enhancing antigen presentation subsets CD8+DCs.

Another in situ vaccination and antitumor therapy study of NaCl NPs in SCC VII bilateral tumor model (FIGS. 8A-8B, Table 4) shows strong immune responses to eliminate cancer cells in the untreated secondary tumors. NaCl NP-treatment to the primary tumor serves as an in situ vaccine to kill the SCC cancer cells and release damage associated molecular patterns (DMAPs), such as ATP and HMGB-1, to recruit antigen-presenting cells (APCs), typically DCs. APCs uptake the dying tumor cells, home to the tumor draining lymph nodes (TDLNs), present the neoantigens on the surface through cross-presentation to T cells. The activated CD8+ T cells further infiltrate to the tumor site and kill the cancer cells in the secondary tumor. NaCl NP-treatment in SCC VII bilateral tumor model shows a significant inhibition of the secondary tumor growth compared to Saline group and untreated group. NaCl NPs treatment induces strong DCs and T cell response, including the reduction of Treg cells (CD4+Foxp3+ T cells), increase of CD8+ T cells, CD8+/Treg ratio and activated DCs.

Example 9 shows that a combination therapy was more effective tumor suppression than PSCNPs or αPD-1 alone (FIGS. 11A-11B).

Collectively, the results presented herein demonstrate a nanoparticle-based approach to alter intracellular osmolarity of cancer cells and kill them. This mechanism may apply to other electrolyte-based nanoparticles, such as KCl and CaCl₂. Unlike molecular ionophores that shuttle one ion at a time (Busschaert et al., Nature Chemistry 9, 667-675 (2017)), PSCNPs translocate millions of sodium and chloride ions into cells. This overwhelms cellular protection mechanism, inducing not only cell apoptosis, but also highly immunogenic necrosis, as a result boosting an anticancer immunity. Menger et al. screened 1040 distinct FDA-approved drugs, and found that cardiac glycosides are particularly efficient ICD inducers (Menger, et al., Sci. Transl. Med., 4, 143ra99 (2012)). Cardiac glycosides resemble PSCNPs and work by inhibiting the cellular sodium potassium ATPase pump and increasing [Na+]int, (Schoner, et al., Am. J. Physiol.: Cell Physiol., 293, C509 (2007).

The ICD property adds to the potential of PSCNPs as a cancer therapeutic. While inorganic nanoparticles have been extensively investigated as imaging probes (Kim, et al., ACS Cent. Sci., 4, 324 (2018), delivery vehicles (Tonga, et al., Curr. Opin. Colloid Interface Sci., 19, 49 (2014), or radiation transducers (Mi, et al., Cancer Nanotechnol., 7, 11 (2016)) few of them have made it to the clinic. The primary concerns were their toxicity, slow clearance, and unpredictable long-term impact to the hosts (Chen, et al., Nat. Rev. Mater., 2, 17024 (2017), Smolkova, et al., Food Chem. Toxicol., 109, 780 (2017), De Matteis, et al., Toxics, 5, 29 (2017)).

Despite extensive research on inorganic nanoparticles, limited attention has been placed on those made from electrolytes. The PSCNPs disclosed herein are made of a benign material and their toxicity is entirely hinged on the nanoparticle form. The assumption is that electrolyte-made nanoparticles are rapidly dissolved in aqueous solutions and behave no different from their constituent salts. These studies indicate otherwise. The discovery introduces a cell killing mechanism and opens up a new perspective on nanoparticle-based therapeutics.

Considering a relatively short half-life in aqueous solutions, PSCNPs local ablation rather than systemic therapy may be preferred. The treatment will cause immediate and immunogenic cancer cell death. After the treatment, the nanoparticles are reduced to salts, which are merged with body's fluid system and cause no systematic or accumulative toxicity. Indeed, no sign of systematic toxicity with i.t. injected PSCNPs at high doses (as discussed in more detail above) were observed.

Because the toxicity is cancer cell selective and temporal, PSCNPs hold great potential in clinical translation as a safe focal treatment modality. For instance, they can be used for pre-operative adjuvant therapy or as a minimally invasive ablation method for patients with inoperable tumors. Particular target cancers include bladder, prostate, head and neck, and liver cancer.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A nanoparticle formed from an alkai metal or alkaline earth metal and halide.

2. The nanoparticle of claim 1 wherein the alkai metal is lithium, sodium, potassium, rubidium, or cesium, and the halide is fluoride, chloride, bromide, or iodide.

3. The nanoparticle of claim 1 wherein alkaline earth metal is magnesium or calcium, and the halide is fluoride, chloride, bromide, or iodide.

4. The nanoparticle of claim 1 comprising sodium chloride, sodium fluoride, sodium bromide, sodium iodide, potassium chloride, or calcium chloride.

5. The nanoparticle of claim 4 comprising sodium chloride.

6. The nanoparticle of claim 4 comprising potassium chloride or calcium chloride.

7. The nanoparticle of claim 5, wherein the molar ratio of sodium and chloride is about 1:1.

8. The nanoparticle of claim 7, wherein the particle is cubic.

9. The nanoparticle of claim 5, further comprising a hydrophilic coating or external layer.

10. The nanoparticle of claim 9, wherein the layer or coating comprises amphiphilic block co-polymers, peptides, proteins, lipids, or a combination thereof.

11. The nanoparticle of claim 10, wherein the layer or coating comprises lipid, such as a phospholipid.

12. The nanoparticle of claim 11, wherein the phospholipid is a phosphoethanolamine.

13. The nanoparticle of claim 9, wherein the layer or coating comprises a PEG such as a PEG amine.

14. The nanoparticle of claim 13, wherein the layer or coating comprises or consists of a lipid-PEG conjugate such as 1,2-distearoyl-sn-glycero-3-phosphoethanolamine (DSPE) PEG (2000) Amine.

15. A pharmaceutical composition comprising a plurality of the nanoparticles of claim 1.

16.-25. (canceled)

26. The pharmaceutical composition of claim 15, comprising an effective amount to nanoparticles to increase apoptosis, necrosis, and/or pyroptosis of tumor and/or cancer cells.

27.-32. (canceled)

33. A method of making antigen comprising contacting cancer cells with an effective amount of the pharmaceutical composition of claim 15 to induce death of the cells.

34.-35. (canceled)

36. The method of claim 33 wherein the contacting occurs in vitro or ex vivo.

37. (canceled)

38. An antigen comprising dying or dead cells, or a lysate, extract, fraction, isolate, or collection of secreted factors thereof formed according to the method of claim 36.

39. A method of vaccinating a subject comprising administering a subject in need thereof an effective amount of the antigen of claim 33 to increase or induce an immune response to the antigen, wherein the contacting occur in vivo, following administration of the pharmaceutical composition to the subject.

40. A method of vaccinating a subject comprising administering a subject in need thereof an effective amount of the antigen of claim 38 to increase or induce an immune response to the antigen

41.-45. (canceled)

46. A method of treating cancer comprising administering to a subject in need thereof the pharmaceutical composition of claim 15.

47. The method of claim 46, wherein the pharmaceutical composition induces an immune response to the cancer in the subject.

48.-52. (canceled)

53. The method of claim 46, further comprising administration of one or more additional active agents.

54. The method of claim 53, wherein the one or more additional active agents comprises an immune checkpoint inhibitor, a chemotherapeutic agent, or a combination thereof.

55. The method of claim 54 comprising an immune checkpoint inhibitor selected from PD-1 antagonists, CTLA4 antagonists, and a combination thereof.

56.-60. (canceled)
